SEQUENCE LISTING (24 sequences)

SEQ ID NO: 1 - protein, 299 aa, Homo sapiens
AAVAALQLGLRAAGLGRAPASAAWRSVLRVSPRPGVAWRPSRCGSSAAEASATKAEDDSFLQWVLLLIPVTAFGLGTWQVQRRKWKLNLIAELESRVLAEPVPLPADPMELKNLEYRPVKVRGCFDHSKELYMMPRTMVDPVREAREGGLISSSTQSGAYVVTPFHCTDLGVTILVNRGFVPRKKVNPETRQKGQIEGEVDLIGMVRLTETRQPFVPENNPERNHWHYRDLEAMARITGAEPIFIDANFQSTVPGGPIGGQTRVTLRNEHLQYIVTWYGLSAATSYLWFKKFLRGTPGV

SEQ ID NO: 2 - protein, 300 aa, Homo sapiens
MAAVAALQLGLRAAGLGRAPASAAWRSVLRVSPRPGVAWRPSRCGSSAAEASATKAEDDSFLQWVLLLIPVTAFGLGTWQVQRRKWKLNLIAELESRVLAEPVPLPADPMELKNLEYRPVKVRGCFDHSKELYMMPRTMVDPVREAREGGLISSSTQSGAYVVTPFHCTDLGVTILVNRGFVPRKKVNPETRQKGQIEGEVDLIGMVRLTETRQPFVPENNPERNHWHYRDLEAMARITGAEPIFIDANFQSTVPGGPIGGQTRVTLRNEHLQYIVTWYGLSAATSYLWFKKFLRGTPGV

SEQ ID NO: 3 - DNA, 897 bp, synthetic construct
  source 1..897: mol_type = other DNA
gccgctgtggctgcactccaactcggcctgagagccgcaggactgggcagagcaccggcatcagccgcctggagaagcgtgctcagagtgtccccaaggcccggagtggcatggcgcccatcgagatgcggcagctcagctgcggaagcctccgcgaccaaggccgaagatgactccttcctgcaatgggtgctgctgctgattcccgtgaccgctttcgggctggggacttggcaagtgcagcggcgcaagtggaagctgaacctgattgccgagctggagtctcgcgtgctggcggaacctgtgccgttgcctgccgatccgatggagctgaagaacctggagtaccggcccgtgaaggtccgaggctgcttcgaccattccaaggaactctacatgatgccacgcacgatggtggaccctgtgcgggaagctcgcgaagggggtctgatttcgagctccacccagtcgggagcctatgtcgtcaccccttttcactgtaccgacctcggcgtcacaatccttgtgaatcggggattcgtgcctcgcaagaaagtcaacccggaaacccggcagaagggacagatcgagggcgaagtggatctgatcggcatggtccggctcaccgagactcggcagccgtttgtgcccgagaacaaccccgagcggaaccactggcactaccgggacctggaagccatggccaggatcactggtgccgagccgattttcatcgacgcgaacttccagtccactgtccctgggggacccatcggtggacagacccgcgtgaccctgaggaacgaacacttgcagtacatcgtgacttggtacggactttccgccgccacctcctacctctggttcaaaaagttcctgaggggtactccgggagtg

SEQ ID NO: 4 - DNA, 900 bp, synthetic construct
  source 1..900: mol_type = other DNA
gccgctgtggctgcactccaactcggcctgagagccgcaggactgggcagagcaccggcatcagccgcctggagaagcgtgctcagagtgtccccaaggcccggagtggcatggcgcccatcgagatgcggcagctcagctgcggaagcctccgcgaccaaggccgaagatgactccttcctgcaatgggtgctgctgctgattcccgtgaccgctttcgggctggggacttggcaagtgcagcggcgcaagtggaagctgaacctgattgccgagctggagtctcgcgtgctggcggaacctgtgccgttgcctgccgatccgatggagctgaagaacctggagtaccggcccgtgaaggtccgaggctgcttcgaccattccaaggaactctacatgatgccacgcacgatggtggaccctgtgcgggaagctcgcgaagggggtctgatttcgagctccacccagtcgggagcctatgtcgtcaccccttttcactgtaccgacctcggcgtcacaatccttgtgaatcggggattcgtgcctcgcaagaaagtcaacccggaaacccggcagaagggacagatcgagggcgaagtggatctgatcggcatggtccggctcaccgagactcggcagccgtttgtgcccgagaacaaccccgagcggaaccactggcactaccgggacctggaagccatggccaggatcactggtgccgagccgattttcatcgacgcgaacttccagtccactgtccctgggggacccatcggtggacagacccgcgtgaccctgaggaacgaacacttgcagtacatcgtgacttggtacggactttccgccgccacctcctacctctggttcaaaaagttcctgaggggtactccgggagtgtga

SEQ ID NO: 5 - DNA, 903 bp, synthetic construct
  source 1..903: mol_type = other DNA
gccgctgtggctgcactccaactcggcctgagagccgcaggactgggcagagcaccggcatcagccgcctggagaagcgtgctcagagtgtccccaaggcccggagtggcatggcgcccatcgagatgcggcagctcagctgcggaagcctccgcgaccaaggccgaagatgactccttcctgcaatgggtgctgctgctgattcccgtgaccgctttcgggctggggacttggcaagtgcagcggcgcaagtggaagctgaacctgattgccgagctggagtctcgcgtgctggcggaacctgtgccgttgcctgccgatccgatggagctgaagaacctggagtaccggcccgtgaaggtccgaggctgcttcgaccattccaaggaactctacatgatgccacgcacgatggtggaccctgtgcgggaagctcgcgaagggggtctgatttcgagctccacccagtcgggagcctatgtcgtcaccccttttcactgtaccgacctcggcgtcacaatccttgtgaatcggggattcgtgcctcgcaagaaagtcaacccggaaacccggcagaagggacagatcgagggcgaagtggatctgatcggcatggtccggctcaccgagactcggcagccgtttgtgcccgagaacaaccccgagcggaaccactggcactaccgggacctggaagccatggccaggatcactggtgccgagccgattttcatcgacgcgaacttccagtccactgtccctgggggacccatcggtggacagacccgcgtgaccctgaggaacgaacacttgcagtacatcgtgacttggtacggactttccgccgccacctcctacctctggttcaaaaagttcctgaggggtactccgggagtgtgataa

SEQ ID NO: 6 - DNA, 907 bp, synthetic construct
  source 1..907: mol_type = other DNA
caccgccgctgtggctgcactccaactcggcctgagagccgcaggactgggcagagcaccggcatcagccgcctggagaagcgtgctcagagtgtccccaaggcccggagtggcatggcgcccatcgagatgcggcagctcagctgcggaagcctccgcgaccaaggccgaagatgactccttcctgcaatgggtgctgctgctgattcccgtgaccgctttcgggctggggacttggcaagtgcagcggcgcaagtggaagctgaacctgattgccgagctggagtctcgcgtgctggcggaacctgtgccgttgcctgccgatccgatggagctgaagaacctggagtaccggcccgtgaaggtccgaggctgcttcgaccattccaaggaactctacatgatgccacgcacgatggtggaccctgtgcgggaagctcgcgaagggggtctgatttcgagctccacccagtcgggagcctatgtcgtcaccccttttcactgtaccgacctcggcgtcacaatccttgtgaatcggggattcgtgcctcgcaagaaagtcaacccggaaacccggcagaagggacagatcgagggcgaagtggatctgatcggcatggtccggctcaccgagactcggcagccgtttgtgcccgagaacaaccccgagcggaaccactggcactaccgggacctggaagccatggccaggatcactggtgccgagccgattttcatcgacgcgaacttccagtccactgtccctgggggacccatcggtggacagacccgcgtgaccctgaggaacgaacacttgcagtacatcgtgacttggtacggactttccgccgccacctcctacctctggttcaaaaagttcctgaggggtactccgggagtgtgataa

SEQ ID NO: 7 - DNA, 900 bp, synthetic construct
  source 1..900: mol_type = other DNA
atggccgctgtggctgcactccaactcggcctgagagccgcaggactgggcagagcaccggcatcagccgcctggagaagcgtgctcagagtgtccccaaggcccggagtggcatggcgcccatcgagatgcggcagctcagctgcggaagcctccgcgaccaaggccgaagatgactccttcctgcaatgggtgctgctgctgattcccgtgaccgctttcgggctggggacttggcaagtgcagcggcgcaagtggaagctgaacctgattgccgagctggagtctcgcgtgctggcggaacctgtgccgttgcctgccgatccgatggagctgaagaacctggagtaccggcccgtgaaggtccgaggctgcttcgaccattccaaggaactctacatgatgccacgcacgatggtggaccctgtgcgggaagctcgcgaagggggtctgatttcgagctccacccagtcgggagcctatgtcgtcaccccttttcactgtaccgacctcggcgtcacaatccttgtgaatcggggattcgtgcctcgcaagaaagtcaacccggaaacccggcagaagggacagatcgagggcgaagtggatctgatcggcatggtccggctcaccgagactcggcagccgtttgtgcccgagaacaaccccgagcggaaccactggcactaccgggacctggaagccatggccaggatcactggtgccgagccgattttcatcgacgcgaacttccagtccactgtccctgggggacccatcggtggacagacccgcgtgaccctgaggaacgaacacttgcagtacatcgtgacttggtacggactttccgccgccacctcctacctctggttcaaaaagttcctgaggggtactccgggagtg

SEQ ID NO: 8 - DNA, 903 bp, synthetic construct
  source 1..903: mol_type = other DNA
atggccgctgtggctgcactccaactcggcctgagagccgcaggactgggcagagcaccggcatcagccgcctggagaagcgtgctcagagtgtccccaaggcccggagtggcatggcgcccatcgagatgcggcagctcagctgcggaagcctccgcgaccaaggccgaagatgactccttcctgcaatgggtgctgctgctgattcccgtgaccgctttcgggctggggacttggcaagtgcagcggcgcaagtggaagctgaacctgattgccgagctggagtctcgcgtgctggcggaacctgtgccgttgcctgccgatccgatggagctgaagaacctggagtaccggcccgtgaaggtccgaggctgcttcgaccattccaaggaactctacatgatgccacgcacgatggtggaccctgtgcgggaagctcgcgaagggggtctgatttcgagctccacccagtcgggagcctatgtcgtcaccccttttcactgtaccgacctcggcgtcacaatccttgtgaatcggggattcgtgcctcgcaagaaagtcaacccggaaacccggcagaagggacagatcgagggcgaagtggatctgatcggcatggtccggctcaccgagactcggcagccgtttgtgcccgagaacaaccccgagcggaaccactggcactaccgggacctggaagccatggccaggatcactggtgccgagccgattttcatcgacgcgaacttccagtccactgtccctgggggacccatcggtggacagacccgcgtgaccctgaggaacgaacacttgcagtacatcgtgacttggtacggactttccgccgccacctcctacctctggttcaaaaagttcctgaggggtactccgggagtgtga

SEQ ID NO: 9 - DNA, 906 bp, synthetic construct
  source 1..906: mol_type = other DNA
atggccgctgtggctgcactccaactcggcctgagagccgcaggactgggcagagcaccggcatcagccgcctggagaagcgtgctcagagtgtccccaaggcccggagtggcatggcgcccatcgagatgcggcagctcagctgcggaagcctccgcgaccaaggccgaagatgactccttcctgcaatgggtgctgctgctgattcccgtgaccgctttcgggctggggacttggcaagtgcagcggcgcaagtggaagctgaacctgattgccgagctggagtctcgcgtgctggcggaacctgtgccgttgcctgccgatccgatggagctgaagaacctggagtaccggcccgtgaaggtccgaggctgcttcgaccattccaaggaactctacatgatgccacgcacgatggtggaccctgtgcgggaagctcgcgaagggggtctgatttcgagctccacccagtcgggagcctatgtcgtcaccccttttcactgtaccgacctcggcgtcacaatccttgtgaatcggggattcgtgcctcgcaagaaagtcaacccggaaacccggcagaagggacagatcgagggcgaagtggatctgatcggcatggtccggctcaccgagactcggcagccgtttgtgcccgagaacaaccccgagcggaaccactggcactaccgggacctggaagccatggccaggatcactggtgccgagccgattttcatcgacgcgaacttccagtccactgtccctgggggacccatcggtggacagacccgcgtgaccctgaggaacgaacacttgcagtacatcgtgacttggtacggactttccgccgccacctcctacctctggttcaaaaagttcctgaggggtactccgggagtgtgataa

SEQ ID NO: 10 - DNA, 910 bp, synthetic construct
  source 1..910: mol_type = other DNA
caccatggccgctgtggctgcactccaactcggcctgagagccgcaggactgggcagagcaccggcatcagccgcctggagaagcgtgctcagagtgtccccaaggcccggagtggcatggcgcccatcgagatgcggcagctcagctgcggaagcctccgcgaccaaggccgaagatgactccttcctgcaatgggtgctgctgctgattcccgtgaccgctttcgggctggggacttggcaagtgcagcggcgcaagtggaagctgaacctgattgccgagctggagtctcgcgtgctggcggaacctgtgccgttgcctgccgatccgatggagctgaagaacctggagtaccggcccgtgaaggtccgaggctgcttcgaccattccaaggaactctacatgatgccacgcacgatggtggaccctgtgcgggaagctcgcgaagggggtctgatttcgagctccacccagtcgggagcctatgtcgtcaccccttttcactgtaccgacctcggcgtcacaatccttgtgaatcggggattcgtgcctcgcaagaaagtcaacccggaaacccggcagaagggacagatcgagggcgaagtggatctgatcggcatggtccggctcaccgagactcggcagccgtttgtgcccgagaacaaccccgagcggaaccactggcactaccgggacctggaagccatggccaggatcactggtgccgagccgattttcatcgacgcgaacttccagtccactgtccctgggggacccatcggtggacagacccgcgtgaccctgaggaacgaacacttgcagtacatcgtgacttggtacggactttccgccgccacctcctacctctggttcaaaaagttcctgaggggtactccgggagtgtgataa

SEQ ID NO: 11 - DNA, 1092 bp, synthetic construct
  source 1..1092: mol_type = other DNA
ctgcgtcccggaagcgcccgcggggccgggtgcgatggcggcggtggctgcgttgcagctggggctgcgggcggcggggctgggacgggccccggccagcgccgcctggaggagcgtcctcagggtctccccgcgcccaggggtggcctggaggccaagcagatgtggcagttctgcagcagaagcatctgccacaaaagcggaagatgactcctttcttcagtgggtcctgctcctcatccctgtgactgcctttggcttggggacatggcaggtccagcgtcggaagtggaagctgaacctgattgcagagttggagtccagagttctggctgagcctgtccctctgccagccgacccaatggaactgaaaaatctggagtataggccagtgaaggtcagggggtgctttgaccattccaaggagctgtatatgatgccccggaccatggtggaccctgtccgggaggcccgggagggcggcctcatctcctcctcaactcagagtggggcctatgtggtcactcccttccactgcaccgacctgggagtcaccatcctggtaaatagagggttcgttcccaggaagaaagtgaatcctgaaacccggcagaaaggccagattgagggagaagtggacctcattgggatggtgaggctgacagaaaccaggcagccttttgtccctgagaacaatccagaaaggaaccactggcattatcgagacctggaagctatggccagaatcacaggcgcagagcccatcttcattgatgccaacttccagagcacagtccctggaggacccattggagggcaaaccagagttactctgaggaacgagcatctgcagtacatcgtgacctggtatggactctctgcagctacatcctacctgtggtttaagaaattcctacgtgggacacctggtgtgtgacagatcagctgctgaagccctgtccctggataatgcagtatttcaagactgcctttatgctggatcatgtgctactggtataaagttctggccttctaccttaaatgagctcatgactggttcatcataaaatcctggcttggtttcagtccaaa

SEQ ID NO: 12 - DNA, 169 bp, synthetic construct
  source 1..169: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttc

SEQ ID NO: 13 - DNA, 106 bp, synthetic construct
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 14 - DNA, 164 bp, synthetic construct
  misc_feature 1..164: note = JeT promoter
  source 1..164: mol_type = other DNA
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 15 - DNA, 225 bp, synthetic construct
  source 1..225: mol_type = other DNA
caattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaa

SEQ ID NO: 16 - DNA, 123 bp, synthetic construct
  source 1..123: mol_type = other DNA
tgtttattgcagcttataatggttacaaataaagcaatagcatcacaaatttcacaaataaagcatttttttcactgcattctagttgtggtttgtccaaactcatcaatgtatcttatcatg

SEQ ID NO: 17 - DNA, 589 bp, synthetic construct
  misc_feature 1..589: note = pUC19 origin of replication
  source 1..589: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 18 - DNA, 816 bp, synthetic construct
  source 1..816: mol_type = other DNA
atgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 19 - DNA, 105 bp, synthetic construct
  source 1..105: mol_type = other DNA
cgcggaacccctatttgtttatttttctaaatacattcaaatatgtatccgctcatgagacaataaccctgataaatgcttcaataatattgaaaaaggaagagt

SEQ ID NO: 20 - DNA, 796 bp, synthetic construct
  misc_feature 1..796: note = cBh promoter
  source 1..796: mol_type = other DNA
tacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgga

SEQ ID NO: 21 - DNA, 254 bp, synthetic construct
  source 1..254: mol_type = other DNA
ctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatggcttctgaggcggaaagaaccagct

SEQ ID NO: 22 - DNA, 903 bp, synthetic construct
  source 1..903: mol_type = other DNA
atggcagccgtcgcagcattgcaactaggacttcgcgcagccggtttgggaagggctccagcttccgcggcctggagaagcgtcctgagagtgtccccgaggcctggagtggcttggcgcccgtcaagatgcggatcgtccgctgccgaagccagcgcgaccaaggccgaggacgattccttcctgcaatgggtgctcctcctgatccccgtgactgccttcgggctgggcacttggcaagtgcagcggcggaagtggaagctgaacttgattgcagaactggagtctagagtgctggcagagcctgtgcccctgcctgctgaccccatggaactcaagaacctggagtacaggcctgtgaaagtcaggggttgctttgaccactccaaggaactttacatgatgcccagaaccatggtggaccctgtcagggaagccagagagggtggactcatctcctcctccacccaatcaggagcctatgtggtcacccccttccactgcactgatctgggagtgaccatcctggtcaacaggggatttgtgcccagaaagaaggtcaacccagaaaccaggcagaagggccagattgagggagaggtggaccttattggcatggtcagactgactgaaaccagacagccttttgtgcctgaaaacaacccagagagaaaccactggcactacagggacctggaagccatggccaggatcactggagctgaacccatcttcattgatgccaacttccagtccactgtgcctgggggccccattggtggccagaccagagtgaccctgagaaatgagcatctgcagtacattgtgacttggtatgggctgtcagcagccacctcctacctgtggttcaagaagttcctcagagggaccccaggtgtctaa

SEQ ID NO: 23 - DNA, 326 bp, synthetic construct
  source 1..326: mol_type = other DNA
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacag

SEQ ID NO: 24 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatgg

(19) United States
(12) Patent Application Publication
(10) Pub. No.: US 2026/0022402 A1
(43) Pub. Date: Jan. 22, 2026

(54) RECOMBINANT ADENO-ASSOCIATED VIRAL VECTOR FOR GENE DELIVERY

(71) Applicant: THE BOARD OF REGENTS OF THE UNIVERSITY OF TEXAS SYSTEM, Austin, TX (US)

(72) Inventors: Qinglan Ling, Dallas, TX (US); Steven J. Gray, Southlake, TX (US)

(73) Assignee: THE BOARD OF REGENTS OF THE UNIVERSITY OF TEXAS SYSTEM, Austin, TX (US)

(21) Appl. No.: 19/287,633

(22) Filed: Jul. 31, 2025

Related U.S. Application Data
(63) Continuation-in-part of application No. 19/010,870, filed on Jan. 6, 2025.
Application No. 19/010,870 is a continuation of application No. 17/092,239, filed on Nov. 7, 2020, now Pat. No. 12,188,039.
(60) Provisional application No. 62/932,828, filed on Nov. 8, 2019.

(51) Int. Cl. C12N 15/86; A61P 43/00

(52) U.S. Cl. CPC C12N 15/86; A61P 43/00; C12N 2750/14141

(57) ABSTRACT

The present disclosure provides methods and compositions for the treatment of diseases and genetic disorders linked to SURF1 loss and/or misfunction. The methods and compositions of the present disclosure comprise rAAV vectors and rAAV viral vectors comprising transgene nucleic acid molecules comprising nucleic acid sequences encoding for a SURF1 polypeptide.

### CROSS REFERENCE TO RELATED APPLICATIONS

This application is a Continuation-in-Part Application of U.S. Non-Provisional application Ser. No. 19/010,870, filed Jan. 6, 2025, which is a continuation of U.S. Non-Provisional application Ser. No. 17/092,239, filed Nov. 7, 2020, which claims the benefit of priority to U.S. Provisional Application No. 62/932,828, filed Nov. 8, 2019, all of which are incorporated by reference herein in their entireties for all purposes.

### INCORPORATION BY REFERENCE OF SEQUENCE LISTING

The instant application contains a Sequence Listing which has been submitted in XML and is hereby incorporated by reference in its entirety. Said XML copy, created on Jul. 28, 2025, is named “UTSD.P3720US.CP1_SEQ_LIST.xml” and is about 36,000 bytes in size.

### BACKGROUND

The SURF1 gene encodes the Surfeit locus protein 1 (SURF1) protein. SURF1 deficiency is a recessively inherited mitochondrial disorder and is the most frequent cause of Leigh syndrome (LS) associated with cytochrome c oxidase (COX, complex IV) deficiency.

The protein encoded by SURF1 is a component of the mitochondrial translation regulation assembly intermediate of cytochrome c oxidase complex (MITRAC complex), which is involved in the regulation of cytochrome c oxidase assembly.

Defects in the SURF1 gene are a cause of Leigh syndrome and Charcot-Marie-Tooth disease 4k (CMT4K), severe neurological disorders that are commonly associated with cytochrome c oxidase (complex IV) deficiency and lactic acidosis.

Compositions and methods provided herein solve the problem of a lack of reagents to study the effects of gene replacement in SURF1 genetic disorders. Furthermore, the delivery of a SURF1 gene in SURF1 deficiency patients is therapeutic, which represents a transformative treatment to address unmet medical needs of these patients.

### SUMMARY

The present disclosure relates generally to the field of gene therapy and in particular, to recombinant adeno-associated viral (AAV) vector particles (also known as rAAV viral vectors) comprising transgene nucleic acid molecules encoding for SURF1, their manufacture, and their use to deliver transgenes to treat or prevent a disease or disorder, including SURF1 deficiency, Leigh Syndrome, mitochondrial complex IV deficiency and Charcot-Marie-Tooth disease 4K.

In some aspects, the current disclosure encompasses a transgene nucleic acid molecule, comprising a nucleic acid sequence as set forth in SEQ ID NO: 22, or a sequence at least 90% identical thereto, operably linked to a promoter sequence. In some aspects, the transgene nucleic acid molecule comprises a nucleic acid sequence as set forth in SEQ ID NO: 22. In some aspects, the transgenic nucleic acid molecule encodes a SURF1 polypeptide. In some aspects, the transgene nucleic acid the promoter comprises a JeT promoter, a UsP promoter (JeTI), a Rous sarcoma virus (RSV) LTR promoter, a cytomegalovirus (CMV) promoter, an SV40 promoter, a dihydrofolate reductase promoter, a beta-actin promoter, a phosphoglycerol kinase (PGK) promoter, a U6 promoter, an H1 promoter, a CAG promoter, a hybrid chicken beta-actin promoter, an MeCP2 promoter, an EF1 promoter, a ubiquitous chicken β-actin hybrid (CBh) promoter, a U1a promoter, a U1b promoter, an MeCP2 promoter, an MeP418 promoter, an MeP426 promoter, a minimal MeCP2 promoter, a VMD2 promoter, an mRho promoter, EF1a promoter, Ubc promoter, human β-actin promoter, TRE promoter, Ac5 promoter, Polyhedrin promoter, CaMKIIa promoter, Gal1 promoter, TEF1 promoter, GDS promoter, ADH1 promoter, Ubi promoter, or α-1-antitrypsin (hAAT) promoter. In some aspects, the promoter sequence comprises a nucleic acid sequence set forth in SEQ ID NO: 14 (JeT), SEQ ID NO: 20 (cBh), or SEQ ID NO: 23 (UsP/JeT1). In some aspects, the promoter sequence comprises a nucleic acid sequence set forth in SEQ ID NO: 23.

In some aspects, the transgene nucleic acid is operably linked to a polyA nucleic acid sequence. In some aspects, the poly A sequence comprises a nucleic acid sequence set forth in SEQ ID NO: 21 or SEQ ID NO: 24.

In some aspects, the transgenic nucleic acid molecule comprises, in the 5′ to 3′ direction

a) the promoter sequence, wherein the promoter sequences comprises the nucleic acid sequence set forth in SEQ ID NO: 14 or SEQ ID NO: 23, or a sequence at least about 95% identical thereto; b) the transgene nucleic acid molecule, wherein the transgene nucleic acid molecule comprises a nucleic acid encoding for a SURF1 polypeptide, comprising a nucleic acid sequence as set forth in SEQ ID NO: 22, or a sequence at least about 90% identical thereto; and c) a polyA sequence comprising the nucleic acid sequence set forth in SEQ ID NO: 21 or SEQ ID NO: 24, or a sequence at least about 95% identical thereto. In some particular aspects, the promoter sequence comprises the nucleic acid sequence set forth in SEQ ID NO: 23; the transgene nucleic acid molecule comprises a nucleic acid encoding for a SURF1 polypeptide, comprising a nucleic acid sequence as set forth in SEQ ID NO: 22; and the polyA sequence comprises the nucleic acid sequence set forth in SEQ ID NO: 21, or SEQ ID NO: 24.

In some aspects, administration of the transgenic nucleic acid molecule into a cell of a subject causes at least a 20% increase in cytochrome c oxidase (COX) activity in the cell and/or reduces blood lactate elevation (Δlactate) by at least 20%, or more in at least one cell of the subject. In some aspects, the transgenic nucleic acid molecule is for use in gene therapy of SURF1-related Leigh syndrome.

In some aspects, the current disclosure also encompasses a viral vector comprising a transgenic nucleic acid molecule encoding a SURF1 polypeptide, wherein the transgenic nucleic acid molecule comprises a nucleic acid sequence as set forth in SEQ ID NO: 22, or a nucleic acid sequence at least 90% identical thereto, operably linked to a promoter sequence and a polyA sequence. In some aspects, the viral vector is a rAAV viral vector. In some aspects, the rAAV viral vector comprises a capsid protein selected from an AAVI capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein, or an AAVrh.10 capsid protein. In some aspects, the rAAV capsid protein is an AAV9 capsid protein. In some aspects, the transgenic nucleic acid molecule comprises a nucleic acid sequence as set forth in SEQ ID NO: 22, operably linked to a promoter sequence and a polyA sequence. In some aspects, the viral vector comprises in the 5′ to 3′ direction: a) the promoter sequence, wherein the promoter sequence comprises the nucleic acid sequence set forth in SEQ ID NO: 14 or SEQ ID NO: 23, or a sequence at least about 95% identical thereto; b) the transgene nucleic acid molecule, wherein the transgene nucleic acid molecule comprises a nucleic acid encoding for a SURF1 polypeptide, comprising a nucleic acid sequence as set forth in SEQ ID NO: 22, or a sequence at least about 90% identical thereto; and c) the poly A sequence comprising, wherein the polyA sequence comprises the nucleic acid sequence set forth in SEQ ID NO: 21, or SEQ ID NO: 24, or a sequence at least about 95% identical thereto. In some aspects, the viral vector comprises, in the 5′ to 3′ direction: a) a first AAV inverted terminal repeat (ITR) sequence; b) the promoter sequence; c) the transgene nucleic acid molecule; d) the polyA sequence; and e) a second AAV ITR sequence, wherein the vector is packaged in an AAV9 capsid.

In some aspects, the current disclosure also encompasses a method of partially or fully restoring SURF1 gene expression in at least one cell of a subject in need thereof, the method comprising administering the viral vector as disclosed herein, to the subject.

In some aspects, administration of the viral vector into the subject restores COX1 activity by at least 20%, or more in at least one cell of the subject, and/or reduces blood lactate elevation (Δlactate) by at least 20%, or more in at least one cell of the subject. In some aspects, the subject has or is exhibiting symptoms of a disease and/or disorder related to SURF1 gene.

In some aspects, disclosed herein is a pharmaceutical composition comprising the transgene nucleic acid disclosed herein and at least one pharmaceutically acceptable excipient and/or additive.

In some aspects, the current disclosure also encompasses a method for treating a subject having a disease and/or disorder related to SURF1 gene, the method comprising administering to the subject a therapeutically effective amount of the pharmaceutical composition disclosed herein. In some aspects, the disease and/or disorder related to SURF1 gene is SURF1 deficiency, Leigh Syndrome, Mitochondrial complex IV deficiency or Charcot-Marie-Tooth disease 4K. In some aspects, the pharmaceutical composition is administered to the subject orally, transmucosally, inhalationally, transdermally, parenterally, intravenously, subcutaneously, intradermally, intramuscularly, intrapleurally, intracerebrally, intrathecally, intracerebrally, intraventricularly, intranasally, intra-aurally, intra-ocularly, or peri-ocularly, topically, intralymphatically, intracisternally, intranervally or intravitreally.

Any of the above aspects, or any other aspect described herein, can be combined with any other aspect.

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings. The patent or application file contains at least one drawing executed in color. Copies of this patent or patent application publication with color drawing(s) will be provided by the Office upon request and payment of the necessary fee.

FIG. 1 is a bacterial plasmid map depicting a codon optimized SURF1 recombinant adeno-associated virus (rAAV) vector.

FIG. 2 is a western blot depicting hSURF1 expression in HEK293 cells transfected with the CBh-hSURF1opt plasmid comprising a codon optimized SURF1 sequence.

FIG. 3 is a series of graphs depicting expression of SURF1 mRNA in multiple brain areas and the spinal cord of mice treated with AAV9 viral vectors comprising a codon optimized SURF1 nucleic acid sequence (AAV9/hSURF1). THM: thalamus, hypothalamus, and midbrain; PMY: pon and medulla; CC: cervical spinal cord; LC: lumbar spinal cord, Low: low IT dose, High: high IT dose, High+IV: combination intrathecal and intravenous dose.

FIGS. 4A-4D are a series of graphs depicting partially restored COX activity deficiency in multiple tissues of SURF1 knockout mice upon treatment with AAV9 viral vectors comprising a codon optimized SURF1 nucleic acid sequence. FIG. 4A depicts COX activity of cerebrum (n=10-18 per group) of WT and SURF1 KO mice with assigned treatments. FIG. 4B depicts cerebellum (n=10-18 per group) of WT and SURF1 KO mice with assigned treatments. FIG. 4C depicts liver (n=5-8 per group) of WT and SURF1 KO mice with assigned treatments. FIG. 4D depicts muscle (n=10-14 per group) of WT and SURF1 KO mice with assigned treatments.

FIG. 5A is a schematic depicting the treatment protocol of impact of AAV9/hSURF1 treatment on serum lactate levels in SURF1 KO mice. FIG. 5B is a graph depicting differences in running time in mice among groups at both 10-weeks-old and 10-months-old. FIG. 5C is a graph depicting change of lactate (ΔLactate) after exercise in mice among groups at both 10-weeks-old and 10-months-old.

FIGS. 6A-6E provide data supporting that AAV9/hSURF1v1 showed cytotoxicity in dorsal root ganglion and myocardium despite minimum immune responses. FIG. 6A shows the study design of GLP toxicology study in WT rats. FIG. 6B shows the average spot count from ELISPOT assay detecting innate immune responses to hSURF1 peptide and AAV9 peptide. FIG. 6C shows the neutralizing antibody (NAb) titer in the serum of the treated rats. FIG. 6D shows that the average severity grades of glial cell hypertrophy and mononuclear cell infiltrate in lumbar dorsal root ganglions (L-DRG). FIG. 6E shows the average severity grades of degeneration/necrosis and mononuclear cell infiltrates in the heart.

FIGS. 7A-7D show that lower SURF1 transgene expression exerted similar improvement in COX activity in patient fibroblast. FIG. 7A shows the designs of newly engineered gene therapy constructs, and the experiment design for B and D. FIG. 7B shows a western blot results from healthy human fibroblast transduced with three constructs in A packaged into AAV2. FIG. 7C shows a plasmid map showing the AAV9/JeT-hSURF1v2-W and AAV9/JeT1-hSURF1v2-M constructs. FIG. 7D shows the COX activity results from healthy human fibroblast and patient fibroblast transduced with three constructs in A packaged into AAV2. Each data point represents measurement from an individual culture flask, with bars representing the Mean±SEM (standard error of the mean). p-values were calculated by comparing with Patient Vehicle group using Tukey's multiple comparisons method following Ordinary Two-way ANOVA.

FIGS. 8A-8D show that AAV9/JeT-hSURF1v2 did not improve COX activity in Surf1 KO mice dosed at PND10 or PND28. FIG. 8A shows the experiment design with three treatment age, two administration routes, and three doses at PND1. FIG. 8B shows COX activity of KO animals treated with vehicle and AAV9/JeT-hSURF1v2. All data were normalized to the average of KO+Vehicle mice. Each data point represents measurement from an individual animal, with bars representing the Mean±SEM (standard error of the mean). p-values were calculated by comparing with KO+Vehicle mice using Tukey's multiple comparisons method following Ordinary one-way ANOVA. FIG. 8C shows a representative image of brain stained with RNAscope. Dots represent positive hSURF1 mRNA expression. FIG. 8D shows Δlactate level of mice treated at PND28, and examined at 10 weeks old and 10 months old. Each data point with error bar represents the Mean±SEM of each experimental group. p-values were calculated by comparing with KO+Vehicle mice using a Tukey's multiple comparison test following two-way ANOVA. N=17-20 per group.

FIGS. 9A-9F. show that AAV9/JeTI-hSURF1v2 improved COX activity in Surf1 KO mice dosed at PND10. FIG. 9A shows relative COX activity in cerebrum. FIG. 9B shows relative COX activity in cerebellum. FIG. 9C shows relative COX activity in liver. FIG. 9D shows relative COX activity in muscle. FIG. 9E shows relative COX activity in heart. All data were normalized to the average of KO+Vehicle group. FIG. 9F shows relative staining area analyzed from RNAscope to examine hSURF1 mRNA expression. Each data point represents measurement from an individual animal, with bars representing the Mean±SEM (standard error of the mean). p-values were calculated by comparing with KO+Vehicle mice using Tukey's multiple comparisons method following Ordinary one-way ANOVA for liver, heart and skeletal muscle. Two-tailed student's t-test was used for cerebrum and cerebellum.

FIGS. 10A-10C show that AAV9/JeTI-hSURF1v2 partially restored endurance capacity and exercise-induced lactate acidosis in Surf1 KO mice dosed at PND10. FIG. 10A shows endurance capacity tested at 10 weeks old and FIG. 10B shows endurance capacity tested at 10 months old. Each data point represents measurement from an individual animal, with bars representing the Mean±SEM (standard error of the mean). FIG. 10C shows Δlactate level of mice treated at PND28, and examined at 10 weeks old and 10 months old. Each data point with error bar represents the Mean±SEM of each experimental group. p-values were calculated by comparing with KO+Vehicle mice using a Tukey's multiple comparison test following two-way ANOVA. N=11-12 per group.

FIG. 11 is a schematic showing AAV9/JeTI-hSURF1v2 did not show toxicity in WT rats. Experiment design and the overall body weight changes of 4 Vehicle-treated rats and 5 AAV9/JeTI-hSUR1v2 treated rats.

FIG. 12 shows gDNA biodistribution of hSURF1 normalized to diploid genome copy number. Each data point represents each treatment group, and the number indicates Mean±SEM (standard error of the mean). Purple shows lower expression and yellow shows higher expression. Color bar is set to log scale.

FIG. 13 shows hSURF1opt mRNA expression level normalized to rat endogenous GAPDH copy number. Each data point represents each treatment group, and the number indicates Mean±SEM (standard error of the mean). Purple shows lower expression and yellow shows higher expression. Color bar is set to log scale.

FIGS. 14A-14B show COX activity comparisons in AAV9/JeT-hSURF1v2 efficacy study. FIG. 14A shows COX activity of cerebrum, cerebellum, liver and skeletal muscle was reduced in KO+Vehicle group compared with WT+Vehicle group. Student's t-tests were performed. FIG. 14B shows AAV9/Jet-hSURF1v2 showed similar improvement in COX activity in cerebrum when injected at different doses through ICV. COX activity of cerebrum from each treatment group. All data were normalized to the average of WT+Vehicle group. Each data point represents measurement from an individual animal, with bars representing the mean±SEM (standard error of the mean). Tukey's multiple comparison method following an ordinary one-way ANOVA were performed. *p-value<0.05; ****p-value<0.0001. The percentage of improvement compared with the KO+Vehicle group from each treatment is provided at the bottom of the respective column.

FIG. 15 shows COX activity comparisons in AAV9/JeTI-hSURF1v2 efficacy study. COX activity of cerebrum, cerebellum, liver and skeletal muscle was reduced in KO+Vehicle group compared with WT+Vehicle group. Student's t-tests were performed.

FIG. 16 shows COX activity from SURF1 KO mice dosed at PND10 with three doses of AAV9/JeTI-hSURF1v2. KO mice were dosed either with vehicle (PBS+5% Sorbitol), or with AAV9/SURF1 vector at 3 different doses of low (1.25E11 vg), mid (2.50E11 vg) or high (5.00E11 vg) per animal. All data were normalized to the average of KO+Vehicle mice. Each data point represents measurement from an individual animal, with bars representing the Mean±SEM (standard error of the mean).

FIGS. 17A-17C show biodistribution and mRNA expression of dose response group of SURF1-/-mice treated at P10. KO mice were dosed either with vehicle (PBS+5% Sorbitol), or with AAV9/SURF1 vector at doses low (1.25E11 vg), mid (2.5E11 vg) or high (5E11 vg) per animal. FIG. 17A shows biodistribution in the brain (striatum, thalamus, midbrain, hind brain). FIG. 17B shows biodistribution in the spinal cord (cervical, thoracic, lumbar) and FIG. 17C biodistribution in the heart, liver, and skeletal muscle (triceps). Spots (example black arrow) show increased SURF1opt transgene mRNA expression in the brain (striatum, thalamus, midbrain, hind brain), spinal cord (cervical, thoracic, lumbar), heart, liver, and skeletal muscle (triceps).

FIGS. 18A-18C show AAV9/JeTI-hSURF1v2 corrected mitochondrial protein expression profile in SURF1 KO mice dosed at PND10. FIG. 18A shows protein expression fold change of SURF1 KO over WT; FC, fold change. Threshold lines (dotted lines) represent FC of 0.67 and 1.5 fold, and a p-value of 0.05 comparing all three groups by one-way ANOVA. FIG. 18B shows protein expression fold change of SURF1 KO treated with JeTI-hSURF1v2 over vehicle treated SURF1 KO. FIG. 18C shows protein expression changes of Cyb5b, Coa3, Ndufa1, Ndufb8, Ndufb9, and Uqcr10. Data represent Mean±SEM (standard error of the mean). P-values were calculated using Tukey's multiple comparisons method following ordinary one-way ANOVA.

### DETAILED DESCRIPTION

### Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present disclosure or the appended claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including,” and “having” are used interchangeably in this disclosure. The terms “comprising,” “including,” and “having” mean to include, but not necessarily be limited to the things so described.

The terms “or” and “and/or,” as used herein, are to be interpreted as inclusive or meaning any one, or any combination. Therefore, “A, B, or C” or “A, B, and/or C” mean any of the following: “A,” “B,” or “C”; “A and B”; “A and C”; “B and C”; “A, B, and C.” An exception to this definition will occur only when a combination of elements, functions, steps, or acts are in some way inherently mutually exclusive.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (3rd ed. 2006); The Cambridge Dictionary of Science and Technology (Walker ed., 1990); The Glossary of Genetics, 5th Ed., R. Rieger et al. (2008), The Harper Collins Dictionary of Biology (1991), all of which are incorporated by reference herein. As used herein, the following terms have the meanings ascribed to them below, unless specified otherwise.

The phraseology and terminology employed herein are for the purpose of description and should not be regarded as limiting. When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as 10% of the value modified by the term “about.” As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

Further, as the present disclosure is susceptible to aspects of many different forms, it is intended that the present disclosure be considered as an example of the principles of the present disclosure and not intended to limit the present disclosure to the specific aspects shown and described. Any one of the features of the present disclosure may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present disclosure may include a variety of combinations and/or integrations of the aspects described herein. Additionally, all aspects of the present disclosure, as described herein, are not essential for its practice. Likewise, other systems, methods, features, and advantages of the present disclosure will be, or become, apparent to one with skill in the art upon examination of the figures and the description. It is intended that all such additional systems, methods, features, and advantages be included within this description, be within the scope of the present disclosure, and be encompassed by the claims.

The term “nucleic acid” refers to deoxyribonucleic acids (DNA), or ribonucleic acids (RNA) and polymers thereof in either single-or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues See, e.g., Batzer et al., Nucleic Acid Res. 19:5081 (1991), the disclosure of which is incorporated in its entirety herein. A “transgene nucleic acid” refers to a nucleic acid molecule—typically DNA, that has been artificially introduced into the an organism, and is not naturally present in that organism's genome. The transgene can originate from a different species (heterologous) or be a modified version of the organism's own gene. This nucleic acid is designed to encode a functional product, such as a protein or RNA. The transgene may be introduced via various methods, including viral vectors, electroporation, or microinjection, and is often incorporated into the host genome in a stable or transient manner. Transgene nucleic acids usually contain regulatory elements, such as promoters and enhancers, to drive expression in specific tissues or under certain conditions.

A “polynucleotide construct” described herein may comprise one or more nucleic acids each encoding a polypeptide, operably linked to a promoter (i.e., in a functional relationship with) and one or more regulatory sequences. Such a polynucleotide construct may alternatively be referred to herein as a “nucleic acid construct” or “construct”.

As used herein, the term “operably linked” refers to a functional linkage between a promoter or other regulatory element and an associated transcribable DNA sequence or coding sequence of a gene (or transgene), such that the promoter, etc., operates to initiate, assist, affect, cause, and/or promote the transcription and expression of the associated transcribable DNA sequence or coding sequence, at least in certain tissue(s), developmental stage(s) and/or condition(s).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

Within the context of the application a protein is represented by an amino acid sequence and correspondingly a nucleic acid molecule or a polynucleotide construct represented by a nucleic acid sequence. Identity and similarity between sequences: throughout this application, each time one refers to a specific amino acid sequence SEQ ID NO (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 65%. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 98%. Another preferred level of sequence identity or similarity is 99%.

Each amino acid sequence described herein by virtue of its identity or similarity percentage with a given amino acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more amino acid (polypeptide or protein) sequences or two or more nucleic acid (polynucleotide construct) sequences, as determined by comparing the sequences. In a preferred aspect, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7, default value is: BLOSUM62 [b162], Gap Open: 1.53, Gap extension: 0.123, Order: aligned, Tree rebuilding number: 2, Guide tree output: ON [true], Max iterate: 2, Perform FFTS: none is used).

“Similarity” between two amino acid sequences is determined by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains.

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and Val to Ile or Leu.

The term “gene therapy” refers to the transfer of genetic material (e.g., DNA or RNA) of interest into a cell to treat or prevent a genetic or acquired disease or condition. The genetic material of interest encodes a product (e.g., a protein polypeptide, peptide or functional RNA) whose production in vivo is desired. For example, the genetic material of interest can encode an enzyme, hormone, receptor, or polypeptide of therapeutic value.

The term “recombinant” as used herein to describe a nucleic acid molecule, means a polynucleotide construct of genomic, cDNA, viral, semisynthetic, and/or synthetic origin, which, by virtue of its origin or manipulation, is not associated with all or a portion of the polynucleotide construct with which it is associated in nature.

As used herein, “treatment,” “therapy,” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition, and/or the remission of the disease, disorder or condition.

As used herein, “prevent,” or “prevention” refers to eliminating or delaying the onset of a particular disease, disorder or physiological condition, or to the reduction of the degree of severity of a particular disease, disorder or physiological condition, relative to the time and/or degree of onset or severity in the absence of intervention.

The term “effective amount,” or “therapeutically effective amount” refers to an amount sufficient to effect beneficial or desirable biological and/or clinical results. The term “therapeutically effective amount,” as used herein, means an amount of a compound or combination of compounds that ameliorates, attenuates, or eliminates one or more symptoms of cancer or prevents or delays the onset of one or more symptoms of cancer as defined herein.

As used herein, “individual,” “subject,” “host,” and “patient” can be used interchangeably herein and refer to any mammalian subject for whom diagnosis, treatment, prophylaxis or therapy is desired, for example, humans, pets, livestock, horses or other animals. As used herein, the term “subject,” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some aspects, the subject can be a human. In other aspects, the subject can be a human in need of treatment for a SURF1 related disease or disorder.

Further, as the present disclosure is susceptible to aspects of many different forms, it is intended that the present disclosure be considered as an example of the principles of the present disclosure and not intended to limit the present disclosure to the specific aspects shown and described. Any one of the features of the present disclosure may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present disclosure may include a variety of combinations and/or integrations of the aspects described herein. Additionally, all aspects of the present disclosure, as described herein, are not essential for its practice. Likewise, other systems, methods, features, and advantages of the present disclosure will be, or become, apparent to one with skill in the art upon examination of the figures and the description. It is intended that all such additional systems, methods, features, and advantages be included within this description, be within the scope of the present disclosure, and be encompassed by the claims.

The present disclosure provides, inter alia, isolated polynucleotides, transgene nucleic acids, recombinant adeno-associated virus (rAAV) vectors, and rAAV viral vectors comprising transgene nucleic acid molecules encoding for SURF1. The present disclosure also provides methods of manufacturing these isolated polynucleotides, rAAV vectors, and rAAV viral vectors, as well as their use to deliver transgenes to treat or prevent a disease or disorder, including diseases associated with loss and/or misfunction of a SURF1 gene. In some aspects, diseases associated with loss and/or misfunction of a SURF1 gene include Leigh Syndrome, Mitochondrial complex IV deficiency and Charcot-Marie-Tooth disease 4K.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types. At least 11 sequentially numbered AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle comprises, consists essentially of, or consists of three major viral proteins: VP1, VP2 and VP3. In some aspects, the AAV refers to the serotype AAV1, AAV2, AAV4, AAV5, AAV6, AAV7, AAV8, AAV9, AAV10, AAV11, AAV12, AAV13, AAVPHP.B, AAVrh74, or AAVrh. 10.

Exemplary adeno-associated viruses and recombinant adeno-associated viruses include, but are not limited to all serotypes (e.g., AAV1, AAV2, AAV3, AAV4, AAV5, AAV6, AAV7, AAV8, AAV9, AAV10, AAV11, AAV12, AAV13, AAVPHP.B, AAVrh74, and AAVrh.10). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03, AAV-KP-1 (described in detail in Kerun et al. JCI Insight, 2019; 4(22):e131610) and AAV-NP59 (described in detail in Paulk et al. Molecular Therapy, 2018; 26(1): 289-303).

### AAV Structure and Function

AAV is a replication-deficient parvovirus, the single-stranded DNA genome of which is about 4.7 kb in length, including two 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC 001862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928, incorporated herein by reference in its entirety. U.S. Pat. No. 9,434,928 also provides the sequences of the capsid proteins and a self-complementary genome. In one aspect, an AVV genome is a self-complementary genome. Cis-acting sequences directing viral DNA replication (rep), encapsidation/packaging, and host cell chromosome integration are contained within AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and encodes the three capsid proteins, VP1, VP2, and VP3. Alternative splicing and non-consensus translational start sites are responsible for the production of the three related capsid proteins. More specifically, after the single mRNA from which each of the VP1, VP2 and VP3 proteins are translated is transcribed, it can be spliced in two different manners: either a longer or shorter intron can be excised, resulting in the formation of two pools of mRNAs: a 2.3 kb-and a 2.6 kb-long mRNA pool. The longer intron is often preferred and thus the 2.3-kb-long mRNA can be called the major splice variant. This form lacks the first AUG codon, from which the synthesis of VP1 protein starts, resulting in a reduced overall level of VP1 protein synthesis. The first AUG codon that remains in the major splice variant is the initiation codon for the VP3 protein. However, upstream of that codon in the same open reading frame lies an ACG sequence (encoding threonine) which is surrounded by an optimal Kozak (translation initiation) context. This contributes to a low level of synthesis of the VP2 protein, which is actually the VP3 protein with additional N terminal residues, as is VP1, as described in Becerra S P et al., (December 1985). “Direct mapping of adeno-associated virus capsid proteins B and C: a possible ACG initiation codon”. Proceedings of the National Academy of Sciences of the United States of America. 82 (23): 7919-23, Cassinotti P et al., (November 1988). “Organization of the adeno-associated virus (AAV) capsid gene: mapping of a minor spliced mRNA coding for virus capsid protein 1”. Virology. 167 (1): 176-84, Muralidhar S et al., (January 1994). “Site-directed mutagenesis of adeno-associated virus type 2 structural protein initiation codons: effects on regulation of synthesis and biological activity”. Journal of Virology. 68 (1): 170-6, and Trempe J P, Carter B J (September 1988). “Alternate mRNA splicing is required for synthesis of adeno-associated virus VP1 capsid protein”. Journal of Virology. 62 (9): 3356-63, each of which is herein incorporated by reference. A single consensus poly A site is located at map position 95 of the AAV genome. The life cycle and genetics of AAV are reviewed in Muzyczka, Current Topics in Microbiology and Immunology, 158:97-129 (1992).

Each VP1 protein contains a VP1 portion, a VP2 portion and a VP3 portion. The VP1 portion is the N-terminal portion of the VP1 protein that is unique to the VP1 protein. The VP2portion is the amino acid sequence present within the VP1 protein that is also found in the N-terminal portion of the VP2 protein. The VP3 portion and the VP3 protein have the same sequence. The VP3 portion is the C-terminal portion of the VP1 protein that is shared with the VP1 and VP2 proteins.

The VP3 protein can be further divided into discrete variable surface regions I-IX (VR-I-IX). Each of the variable surface regions (VRs) can comprise or contain specific amino acid sequences that either alone or in combination with the specific amino acid sequences of each of the other VRs can confer unique infection phenotypes (e.g., decreased antigenicity, improved transduction and/or tissue-specific tropism relative to other AAV serotypes) to a particular serotype as described in DiMatta et al., “Structural Insight into the Unique Properties of Adeno-Associated Virus Serotype 9” J. Virol., Vol. 86 (12): 6947-6958 June 2012, the contents of which are incorporated herein by reference.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8:659-669 (1997); Kessler et al., Proc Nat. Acad Sc. USA, 93:14082-14087 (1996); and Xiao et al., J Virol, 70:8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94:5804-5809 (1997) and Murphy et al., Proc Natl Acad Sci USA, 94:13921-13926 (1997). Moreover, Lewis et al., J Virol, 76:8769-8775 (2002) demonstrated that skeletal myofibers possess the necessary cellular factors for correct antibody glycosylation, folding, and secretion, indicating that muscle is capable of stable expression of secreted protein therapeutics. Recombinant AAV (rAAV) genomes of the invention comprise, consist essentially of, or consist of a nucleic acid molecule encoding a therapeutic protein (e.g., SURF1) and one or more AAV ITRs flanking the nucleic acid molecule. AAV DNA in the rAAV genomes may be from any AAV serotype for which a recombinant virus can be derived including, but not limited to, AAV serotypes AAV1, AAV2, AAV3, AAV4, AAV5, AAV6, AAV7, AAV8, AAV9, AAV10, AAV11, AAV12, AAV13, AAVPHP.B, AAVrh74, and AAVrh.10. Production of pseudotyped rAA Vis disclosed in, for example, WO2001083692. Other types of rAAV variants, for example rAAV with capsid mutations, are also contemplated. See, e.g., Marsic et al., Molecular Therapy, 22 (11): 1900-1909 (2014). The nucleotide sequences of the genomes of various AAV serotypes are known in the art.

### Transgene Nucleic Acids and Polypeptides

The present disclosure provides isolated polynucleotides comprising at least one transgene nucleic acid molecule.

In some aspects, a transgene nucleic acid molecule can comprise a nucleic acid sequence encoding a SURF1 polypeptide, or at least one fragment thereof. As would be appreciated by the skilled artisan, SURF1 is encoded for by the SURF1 gene in the human genome. Thus, a transgene nucleic acid molecule can comprise, consist essentially of, or consist of an SURF1 sequence, or any fragment thereof. In some aspects, a transgene nucleic acid molecule can comprise a nucleic acid sequence encoding a biological equivalent of a SURF1 polypeptide.

In some aspects, a SURF1 polypeptide comprises, consists essentially of, or consists of an amino acid sequence at least 65%, 70%, 75%, 80%, 85%, 90%, 95%, 96%, 97%, 98%, 99% or 100% (or any percentage in between) identical to the amino acid sequence put forth in SEQ ID NO: 1 or SEQ ID NO: 2.

In some aspects, a SURF1 polypeptide comprises, consists essentially of, or consists of an amino acid sequence at least 65%, 70%, 75%, 80%, 85%, 90%, 95%, 96%, 97%, 98%, 99% or 100% (or any percentage in between) identical to at least one portion of the amino acid sequence put forth in SEQ ID NO: 1 or SEQ ID NO: 2.

In some aspects, a nucleic acid sequence encoding a SURF1 polypeptide comprises, consists essentially of, or consists of a nucleic acid sequence at least 65%, 70%, 75%, 80%, 85%, 90%, 95%, 96%, 97%, 98%, 99% or 100% (or any percentage in between) identical to any one of the nucleic acid sequences put forth in SEQ ID NOs: 3-10 or SEQ ID NO: 22. In some aspects, a nucleic acid sequence encoding a SURF1 polypeptide comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleic acid sequences put forth in SEQ ID NOs: 10. A nucleic acid sequence encoding a SURF1 polypeptide can be referred to as a SURF1 sequence.

In some aspects, a nucleic acid sequence encoding a SURF1 polypeptide comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleic acid sequences put forth in SEQ ID NO: 22.

In some aspects, the nucleic acid sequence encoding a SURF1 polypeptide can be a codon optimized nucleic acid sequence that encodes for a SURF1 polypeptide. A codon optimized nucleic acid sequence encoding a SURF1 polypeptide can comprise, consist essentially of, or consist of a nucleic acid sequence that is no more than 65%, 70%, 75%, 80%, 85%, 90%, 95%, 96%, 97%, 98%, 99% (or any percentage in between) identical to the wildtype human nucleic acid sequence encoding the SURF1 polypeptide. As used herein, the “wildtype human nucleic acid sequence of the SURF1 polypeptide” refers to the nucleic acid sequence that encodes the SURF1 polypeptide in a human genome, as put forth in SEQ ID NO: 11.

SEQ ID NOs: 3-10 and SEQ ID NO: 22 are unique codon optimized nucleic acid sequences that encode for a SURF1 polypeptide.

In some aspects, a codon optimized nucleic acid sequence encoding a SURF1 polypeptide such as those put forth in SEQ ID NOs: 3-10 or SEQ ID NO: 22, can comprise no donor splice sites. In some aspects, a codon optimized nucleic acid sequence encoding a SURF1 polypeptide can comprise no more than about one, or about two, or about three, or about four, or about five, or about six, or about seven, or about eight, or about nine, or about ten donor splice sites. In some aspects, a codon optimized nucleic acid sequence encoding a SURF1 polypeptide comprises at least one, or at least two, or at least three, or at least four, or at least five, or at least six, or at least seven, or at least eight, or at least nine, or at least ten fewer donor splice sites as compared to the wildtype human nucleic acid sequence the SURF1 polypeptide. Without wishing to be bound by theory, the removal of donor splice sites in the codon optimized nucleic acid sequence can unexpectedly and unpredictably increase expression of the SURF1 polypeptide in vivo, as cryptic splicing is prevented. Moreover, cryptic splicing may vary between different subjects, meaning that the expression level of the SURF1 polypeptide comprising donor splice sites may unpredictably vary between different subjects. Such unpredictability is unacceptable in the context of human therapy. Accordingly, the codon optimized nucleic acid sequences put forth in SEQ ID NOs: 3-10 or SEQ ID NO: 22, which lacks donor splice sites, unexpectedly and surprisingly allows for increased expression of the SURF1 polypeptide in human subjects and regularizes expression of the SURF1 polypeptide across different human subjects.

In some aspects, a codon optimized nucleic acid sequence encoding a SURF1 polypeptide, such as those put forth in SEQ ID NOs: 3-10 or SEQ ID NO: 22, can have a GC content that differs from the GC content of the wildtype human nucleic acid sequence the SURF1 polypeptide. In some aspects, the GC content of a codon optimized nucleic acid sequence encoding a SURF1 polypeptide is more evenly distributed across the entire nucleic acid sequence, as compared to the wildtype human nucleic acid sequence the SURF1 polypeptide. Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects, a codon optimized nucleic acid sequence encoding a SURF1 polypeptide, such as those put forth in SEQ ID NOs: 3-10 or SEQ ID NO: 22, can have fewer repressive microRNA target binding sites as compared to the wildtype human nucleic acid sequence the SURF1 polypeptide. In some aspects, a codon optimized nucleic acid sequence encoding a SURF1 polypeptide can have at least one, or at least two, or at least three, or at least four, or at least five, or at least six, or at least seven, or at least eight, or at least nine, or at least ten, or at least ten fewer repressive microRNA target binding sites as compared to the wildtype human nucleic acid sequence the SURF1 polypeptide. Without wishing to be bound by theory, by having fewer repressive microRNA target binding sites, the codon optimized nucleic acid sequence encoding a SURF1 polypeptide unexpectedly exhibits increased expression in a human subject.

In some aspects, the codon optimized nucleic acid sequence encoding a SURF1 polypeptide exhibits at least 5%, at least 10%, at least 20%, at least 30%, at least 50%, at least 75%, at least 100%, at least 200%, at least 300%, at least 500%, or at least 1000% increased expression in a human subject relative to a wild-type or non-codon optimized nucleic acid sequence encoding a SURF1 polypeptide. In some aspects, the codon optimized nucleic acid sequence encoding a SURF1 polypeptide exhibits moderate to weak expression of SURF1 polypeptide as provided in Examples 6.

Some exemplary embodiments vis-à-vis transgene nucleic acids of the current disclosure are described below:
1. A transgene nucleic acid molecule, comprising a nucleic acid sequence as set forth in SEQ ID NO: 22, or a sequence at least 90% identical thereto, operably linked to a promoter sequence.
2. The transgenic nucleic acid molecule of embodiment 1, wherein the transgene nucleic acid molecule comprises a nucleic acid sequence as set forth in SEQ ID NO: 22.
3. The transgenic nucleic acid molecule of embodiment 1, wherein the transgenic nucleic acid molecule encodes a SURF1 polypeptide.
4. The transgene nucleic acid molecule of embodiment 1, wherein the promoter comprises a JeT promoter, a UsP promoter (JeTI), a Rous sarcoma virus (RSV) LTR promoter, a cytomegalovirus (CMV) promoter, an SV40 promoter, a dihydrofolate reductase promoter, a beta-actin promoter, a phosphoglycerol kinase (PGK) promoter, a U6 promoter, an H1 promoter, a CAG promoter, a hybrid chicken beta-actin promoter, an MeCP2 promoter, an EF1 promoter, a ubiquitous chicken β-actin hybrid (CBh) promoter, a U1a promoter, a U1b promoter, an MeCP2 promoter, an MeP418 promoter, an MeP426 promoter, a minimal MeCP2 promoter, a VMD2 promoter, an mRho promoter, EF1a promoter, Ubc promoter, human β-actin promoter, TRE promoter, Ac5 promoter, Polyhedrin promoter, CaMKIIa promoter, Gal1 promoter, TEF1 promoter, GDS promoter, ADH1 promoter, Ubi promoter, or α-1-antitrypsin (hAAT) promoter.
5. The transgene nucleic acid of embodiment 4, wherein the promoter sequence comprises a nucleic acid sequence set forth in SEQ ID NO: 14 (JeT), SEQ ID NO: 20 (cBh), or SEQ ID NO: 23 (UsP/JeT1).
6. The transgene nucleic acid of embodiment 5, wherein the promoter sequence comprises a nucleic acid sequence set forth in SEQ ID NO: 23.
7. The transgene nucleic acid of embodiment 1, further comprising a poly A sequence operably linked to the nucleic acid sequence.
8. The transgene nucleic acid of embodiment 7, wherein the polyA sequence comprises a nucleic acid sequence set forth in SEQ ID NO: 21 or SEQ ID NO: 24.
9. A transgenic nucleic acid molecule of embodiment 1, comprising, in the 5′ to 3′ direction a) the promoter sequence, wherein the promoter sequences comprises the nucleic acid sequence set forth in SEQ ID NO: 14 or SEQ ID NO: 23, or a sequence at least about 95% identical thereto; b) the transgene nucleic acid molecule, wherein the transgene nucleic acid molecule comprises a nucleic acid encoding for a SURF1 polypeptide, comprising a nucleic acid sequence as set forth in SEQ ID NO: 22, or a sequence at least about 90% identical thereto; and c) a polyA sequence comprising the nucleic acid sequence set forth in SEQ ID NO: 21 or SEQ ID NO: 24, or a sequence at least about 95% identical thereto.
10. The transgenic nucleic acid molecule of embodiment 9, wherein the promoter sequence comprises the nucleic acid sequence set forth in SEQ ID NO: 23; the transgene nucleic acid molecule comprises a nucleic acid encoding for a SURF1 polypeptide, comprising a nucleic acid sequence as set forth in SEQ ID NO: 22; and the polyA sequence comprises the nucleic acid sequence set forth in SEQ ID NO: 21, or SEQ ID NO: 24.
11. The transgenic nucleic acid molecule of embodiment 10, wherein administration of the transgenic nucleic acid molecule into a cell of a subject causes at least a 20% increase in cytochrome c oxidase (COX) activity in the cell and/or reduces blood lactate elevation (Δlactate) by at least 20%, or more in at least one cell of the subject.
12. The transgenic nucleic acid molecule of embodiment 10, wherein the transgenic nucleic acid molecule is for use in gene therapy of SURF1-related Leigh syndrome.

### PolyA Sequences

In some aspects, a polyadenylation (polyA) sequence can comprise any polyA sequence known in the art. Non-limiting examples of polyA sequences include, but are not limited to, an MeCP2 polyA sequence, a retinol dehydrogenase 1 (RDH1) polyA sequence, a bovine growth hormone (BGH) polyA sequence, an SV40 polyA sequence, a SPA49 polyA sequence, a sNRP-TK65 poly A sequence, a sNRP polyA sequence, or a TK65 polyA sequence.

Thus, a poly A sequence can comprise, consists essentially of or consist of an MeCP2 poly A sequence, a retinol dehydrogenase 1 (RDH1) polyA sequence, a bovine growth hormone (BGH) polyA sequence, an SV40 polyA sequence, a SPA49 polyA sequence, a sNRP-TK65 polyA sequence, a sNRP polyA sequence, or a TK65 polyA sequence.

In some aspects, a polyA sequence can comprise, consist essentially of, or consist of an SV40pA sequence. In some aspects, an SV40pA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 16.

In some aspects, a polyA sequence can comprise, consist essentially of, or consist of a BGHpA sequence. In some aspects, an BGHpA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NO: 21, or SEQ ID NO: 24.

### Viral Vectors

In some aspects, the transgene nucleic acid and polynucleotide constructs disclosed herein may be comprised within a vector for delivery, and/or may be delivered in a delivery particle. Vectors comprising the transgene nucleic acid and polynucleotide constructs according to the present disclosure include all those known in the art, including cosmids, plasmids (e.g., naked or contained in liposomes), and viral constructs (e.g., lentiviral, retroviral, adenoviral, and adeno associated viral constructs) that can incorporate the transgene nucleic acid or a polynucleotide construct encoding a polypeptide described herein, or a variant thereof. Those of skill in the art will be capable of selecting suitable constructs, as well as cells, for making any of the polynucleotide constructs described herein. In some aspects, a construct is a plasmid (i.e., a circular DNA molecule that can autonomously replicate inside a cell). In some aspects, a construct can be a cosmid (e.g., pWE or sCos series).

In some aspects, a vector is a viral vector. In some aspects, a viral construct is a lentivirus, retrovirus, adenovirus, or adeno-associated virus construct. In some aspects, a construct is an adeno-associated virus (AAV) construct. In some aspects, a viral construct is an adenovirus construct. In some aspects, a viral construct may also be based on or derived from an alphavirus. Pseudotyped viruses may be formed by combining alphaviral envelope glycoproteins and retroviral capsids.

In some aspects, constructs provided herein can be of different sizes. In some aspects, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some aspects, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb. In some aspects, a construct is a viral construct and can have a total number of nucleotides

of up to 10 kb. In some aspects, a viral construct can have a total number of nucleotides in the range of about 4.5 kb to 5 kb, or about 4.7 kb. In some aspects, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 1 0 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some aspects, a construct is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb.

In some aspects, a construct is an adeno-associated virus construct and can have a total number of nucleotides of up to 8 kb. In some aspects, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the constructs described herein can further include a control sequence, e.g., a control sequence selected from the group of a transcription initiation sequence, a transcription termination sequence, a promoter sequence, an enhancer sequence, an RNA splicing sequence, a polyadenylation (poly (A)) sequence, a Kozak consensus sequence, and/or additional untranslated regions which may house pre-or post-transcriptional regulatory and/or control elements. In some aspects, a promoter can be a native promoter, a constitutive promoter, an inducible promoter, and/or a tissue-specific promoter. Non-limiting examples of control sequences are described herein.

### AAV Vectors

In some aspects, the isolated polynucleotides comprising at least one transgene nucleic acid sequence described herein can be a recombinant AAV (rAAV) vector.

As used herein, the term “vector” refers to a nucleic acid comprising, consisting essentially of, or consisting of an intact replicon such that the vector may be replicated when placed within a cell, for example by a process of transfection, infection, or transformation. It is understood in the art that once inside a cell, a vector may replicate as an extrachromosomal (episomal) element or may be integrated into a host cell chromosome. Vectors may include nucleic acids derived from retroviruses, adenoviruses, herpesvirus, baculoviruses, modified baculoviruses, papovaviruses, or otherwise modified naturally-occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising, consisting essentially of, or consisting of DNA condensed with cationic polymers such as heterogeneous polylysine, defined-length oligopeptides, and polyethyleneimine, in some cases contained in liposomes; and the use of ternary complexes comprising, consisting essentially of, or consisting of a virus and polylysine-DNA.

With respect to general recombinant techniques, vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif) and Promega Biotech (Madison, Wis.). In order to optimize expression and/or in vitro transcription, it may be necessary to remove, add or alter 5′ and/or 3′ untranslated portions of cloned transgenes to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

An “rAAV vector” as used herein refers to a vector comprising, consisting essentially of, or consisting of one or more transgene nucleic acid molecules and one or more AAV inverted terminal repeat sequences (ITRs). Such AAV vectors can be replicated and packaged into infectious viral particles when present in a host cell that provides the functionality of rep and cap gene products; for example, by transfection of the host cell. In some aspects, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing rAAV vectors may also contain elements for manufacturing purposes, e.g., antibiotic resistance genes, origin of replication sequences etc., but these are not encapsidated and thus do not form part of the AAV particle.

In some aspects, an rAAV vector can comprise at least one transgene nucleic acid molecule. In some aspects, an rAAV vector can comprise at least one AAV inverted terminal (ITR) sequence. In some aspects, an rAAV vector can comprise at least one promoter sequence. In some aspects, an rAAV vector can comprise at least one enhancer sequence. In some aspects, an rAAV vector can comprise at least one polyA sequence. In some aspects, an rAAV vector can comprise at least one origin of replication sequence. In some aspects, an rAAV vector can comprise at least one self-cleaving peptide sequence. In some aspects, an rAAV vector can comprise at least one antibiotic resistance gene. In some aspects, an rAAV vector can comprise at least one reporter protein.

In some aspects, an rAAV vector can comprise a first AAV ITR sequence, a promoter sequence, a transgene nucleic acid molecule, a polyA sequence, and a second AAV ITR sequence. In some aspects, an rAAV vector can comprise, in the 5′ to 3′ direction, a first AAV ITR sequence, a promoter sequence, a transgene nucleic acid molecule, a polyA sequence, and a second AAV ITR sequence.

In some aspects, an rAAV vector can comprise more than one transgene nucleic acid molecule. In some aspects, an rAAV vector can comprise at least two transgene nucleic acid molecule, such that the rAAV vector comprises a first transgene nucleic acid molecule and an at least second transgene nucleic acid molecule. In some aspects, the first and the at least second transgene nucleic acid molecules can comprise the same sequence. In some aspects, the first and the at least second transgene nucleic acid molecules can comprise different sequence. In some aspects, the first and the at least second transgene nucleic acid molecules can be adjacent to each other. In some aspects, the first and the at least second transgene nucleic acid molecules can be separated by at least one self-cleaving peptide sequence.

In some aspects, an rAAV vector can comprise more than one promoter sequence. In some aspects, an rAAV vector can comprise at least two promoter sequences, such that the rAAV vector comprises a first promoter sequence and an at least second promoter sequence. In some aspects, the first and the at least second promoter sequences can comprise the same sequence. In some aspects, the first and the at least second promoter sequences can comprise different sequences. In some aspects, the first and the at least second promoter sequences can be adjacent to each other. In some aspects wherein an rAAV vector also comprises a first transgene nucleic acid molecule and an at least second transgene nucleic acid molecule, the first promoter can be located upstream (5′) of the first transgene nucleic acid molecule and the at least second promoter can be located between the first transgene nucleic acid molecule and the at least second transgene nucleic acid molecule, such that the at least second promoter is downstream (3′) of the first transgene nucleic acid molecule and upstream (5′) of the at least second transgene nucleic acid molecule.

Any of the preceding rAAV vectors can further comprise at least one enhancer. The at least one enhancer can be located anywhere in the rAAV vector. In some aspects, the at least one enhancer can be located immediately upstream (5′) of a promoter. Thus, an rAAV vector can comprise, in the 5′ to 3′ direction, a first AAV ITR sequence, an enhancer, a promoter sequence, a transgene nucleic acid molecule, a polyA sequence, and a second AAV ITR sequence. In some aspects, the at least one enhancer can be located immediately downstream (3′) of a promoter. Thus, an rAAV vector can comprise, in the 5′ to 3′ direction, a first AAV ITR sequence, a promoter sequence, an enhancer, a transgene nucleic acid molecule, a polyA sequence, and a second AAV ITR sequence. In some aspects, the at least one enhancer can be located immediately downstream of a transgene nucleic acid molecule. Thus, an rAAV vector can comprise, in the 5′ to 3′ direction, a first AAV ITR sequence, a promoter sequence, a transgene nucleic acid molecule, an enhancer, a polyA sequence, and a second AAV ITR sequence.

### AAV ITR Sequences

In some aspects, an AAV ITR sequence can comprise any AAV ITR sequence known in the art. In some aspects, an AAV ITR sequence can be an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence, or an AAVrh.10 ITR sequence.

Thus, in some aspects, an AAV ITR sequence can comprise, consist essentially of, or consist of an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence, or an AAVrh.10 ITR sequence.

In some aspects, an rAAV vector of the present disclosure can comprise, consist essentially of, or consist of AAV2 ITR sequences. In some aspects, an rAAV vector of the present disclosure can comprise, consist essentially of, or consist of AAV2 ITR sequences or a modified AAV2 ITR sequence.

In some aspects, an AAV2 ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 12.

In some aspects, a modified AAV2 ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 13.

In some aspects, a first AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 12 and a second AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 13.

In some aspects, a first AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 13 and a second AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 12.

### Promoter Sequence and Enhancers

The term “promoter” and “promoter sequence” as used herein means a control sequence that is a region of a polynucleotide sequence at which the initiation and rate of transcription of a coding sequence, such as a gene or a transgene, are controlled. Promoters may be constitutive, inducible, repressible, or tissue-specific, for example. Promoters may contain genetic elements at which regulatory proteins and molecules such as RNA polymerase and transcription factors may bind. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a β-actin promoter, a phosphoglycerol kinase (PGK) promoter, a U6 promoter, an H1 promoter, a ubiquitous chicken β-actin hybrid (CBh) promoter, a small nuclear RNA (U1a or U1b) promoter, an MeCP2 promoter, an MeP418 promoter, an MeP426 promoter, a minimal MeCP2 promoter, a VMD2 promoter, an mRho promoter EF1 promoter, a JeT promoter, or a JeTI (USP) promoter. In some aspects, the promoter is a JeT promoter, or a JeTI (USP) promoter.

Additional non-limiting exemplary promoters provided herein include, but are not limited to EF1a, Ubc, human β-actin, CAG, TRE, Ac5, Polyhedrin, CaMKIIa, Gal1, TEF1, GDS, ADH1, Ubi, and α-1-antitrypsin (hAAT). It is known in the art that the nucleotide sequences of such promoters may be modified in order to increase or decrease the efficiency of mRNA transcription. See, e.g., Gao et al. (2018) Mol. Ther.: Nucleic Acids 12:135-145 (modifying TATA box of 7SK, U6 and H1 promoters to abolish RNA polymerase III transcription and stimulate RNA polymerase II-dependent mRNA transcription). Synthetically-derived promoters may be used for ubiquitous or tissue specific expression. Further, virus-derived promoters, some of which are noted above, may be useful in the methods disclosed herein, e.g., CMV, HIV, adenovirus, and AAV promoters. In some aspects, the promoter is used together with at least one enhancer to increase the transcription efficiency. Non-limiting examples of enhancers include an interstitial retinoid-binding protein (IRBP) enhancer, an RSV enhancer or a CMV enhancer.

In some aspects, a promoter sequence can comprise, consist essentially of, or consist of a Rous sarcoma virus (RSV) LTR promoter sequence (optionally with the RSV enhancer), a cytomegalovirus (CMV) promoter sequence, an SV40 promoter sequence, a dihydrofolate reductase promoter sequence, a β-actin promoter sequence, a phosphoglycerol kinase (PGK) promoter sequence, a U6 promoter sequence, an H1 promoter sequence, a ubiquitous chicken β-actin hybrid (CBh) promoter sequence, a small nuclear RNA (U1a or U1b) promoter sequence, an MeCP2 promoter sequence, an MeP418 promoter sequence, an MeP426 promoter sequence, a minimal MeCP2 promoter sequence, a VMD2 promoter sequence, an mRho promoter sequence, an EFI promoter sequence, an EF1a promoter sequence, a Ubc promoter sequence, a human β-actin promoter sequence, a CAG promoter sequence, a TRE promoter sequence, an Ac5 promoter sequence, a Polyhedrin promoter sequence, a CaMKIIa promoter sequence, a Gal1 promoter sequence, a TEF1 promoter sequence, a GDS promoter sequence, an ADH1 promoter sequence, a Ubi promoter sequence or an α-1-antitrypsin (hAAT) promoter sequence.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter. Non-limiting examples of linked enhancer/promoter for use in the methods, compositions and constructs provided herein include a PDE promoter plus IRBP enhancer or a CMV enhancer plus U1a promoter. It is understood in the art that enhancers can operate from a distance and irrespective of their orientation relative to the location of an endogenous or heterologous promoter. It is thus further understood that an enhancer operating at a distance from a promoter is thus “operably linked” to that promoter irrespective of its location in the vector or its orientation relative to the location of the promoter.

As used throughout the disclosure, the term “operably linked” refers to the expression of a gene (i.e. a transgene) that is under the control of a promoter with which it is spatially connected. A promoter can be positioned 5′ (upstream) or 3′ (downstream) of a gene under its control. A promoter can be positioned 5′ (upstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

In some aspects, a promoter sequence can comprise, consist essentially of, or consist of a JeT promoter sequence. A JeT promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 14.

In some aspects, a promoter sequence can comprise, consist essentially of, or consist of a MeP229 promoter sequence. A meP229 promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 15.

In some aspects, a promoter sequence can comprise, consist essentially of, or consist of a CBh promoter sequence. A CBh promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 20.

In some aspects, a promoter sequence can comprise, consist essentially of, or consist of a JeTI (UsP) promoter sequence. A JeTI (UsP) promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 23.

In some aspects, bacterial plasmids of the present disclosure can comprise a prokaryotic promoter.

A prokaryotic promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 19.

In some aspects, bacterial plasmid, or the viral vector, for example the rAAV described herein, may comprise the transgene nucleic acid described herein operably linked to the promoter sequence. In some aspects, a transgene nucleic acid molecules can comprise a nucleic acid molecule encoding a SURF1 polypeptide, or at least one fragment thereof. In some aspects, a transgene nucleic acid molecule can comprise a nucleic acid sequence encoding a biological equivalent of a SURF1 polypeptide, or at least one fragment thereof.

In some aspects, a SURF1 polypeptide comprises, consists essentially of, or consists of an amino acid sequence at least 65%, 70%, 75%, 80%, 85%, 90%, 95%, 96%, 97%, 98%, 99% or 100% (or any percentage in between) identical to the amino acid sequence put forth in SEQ ID NO: 1 or SEQ ID NO: 2, or a fragment thereof. In some aspects, a SURF1 polypeptide comprises, consists essentially of, or consists of an amino acid sequence at least 65%, 70%, 75%, 80%, 85%, 90%, 95%, 96%, 97%, 98%, 99% or 100% (or any percentage in between) identical to at least one portion of the amino acid sequence put forth in SEQ ID NO: 1, or a fragment thereof.

In some aspects, a nucleic acid sequence encoding a SURF1 polypeptide comprises, consists essentially of, or consists of a nucleic acid sequence at least 65%, 70%, 75%, 80%, 85%, 90%, 95%, 96%, 97%, 98%, 99% or 100% (or any percentage in between) identical to any one of the nucleic acid sequences put forth in SEQ ID NOs: 3-10 or SEQ ID NO: 22. In some aspects, a nucleic acid sequence encoding a SURF1 polypeptide comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleic acid sequences put forth in SEQ ID NOs: 6. In some aspects, a nucleic acid sequence encoding a SURF1 polypeptide comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleic acid sequences put forth in SEQ ID NOs: 22. A nucleic acid sequence encoding a SURF1 polypeptide can be referred to as a SURF1 nucleic acid molecule.

In some aspects, a nucleic acid sequence encoding a SURF1 polypeptide can be a codon optimized nucleic acid sequence that encodes for a SURF1 polypeptide. A codon optimized nucleic acid sequence encoding a SURF1 polypeptide can comprise, consist essentially of, or consist of a nucleic acid sequence that is no more than 65%, 70%, 75%, 80%, 85%, 90%, 95%, 96%, 97%, 98%, 99% (or any percentage in between) identical to the wildtype human nucleic acid sequence encoding the SURF1 polypeptide. As used herein, the “wildtype human nucleic acid sequence encoding the SURF1 polypeptide” refers to the nucleic acid sequence that encodes the SURF1 polypeptide in a human genome, as put forth in, for example, SEQ ID NO: 11.

SEQ ID NOs: 3-10 and SEQ ID NO: 22 are unique codon optimized nucleic acid sequences that encode for a SURF1 polypeptide.

In some aspects, a transgene nucleic acid molecule can comprise, consist essentially of, or consist of a nucleic acid sequence encoding a reporter protein. As used herein, a reporter protein is a detectable protein that is operably linked to a promoter to assay the expression (for example, tissue specificity and/or strength) of the promoter. In aspects, a reporter protein may be operably linked to a polypeptide. In aspects, reporter proteins may be used in monitoring DNA delivery methods, functional identification and characterization of promoter and enhancer elements, translation and transcription regulation, mRNA processing and protein: protein interactions. Non-limiting examples of a reporter protein are β-galactosidase; a fluorescent protein, such as, Green Fluorescent Protein (GFP) or Red Fluorescent Protein (RFP); luciferase; glutathione S-transferase; and maltose binding protein.

In some aspects, a transgene nucleic acid molecule can further comprise a nucleic acid sequence encoding a signal peptide.

In some aspects, the SURF1 polypeptide can comprise a signal peptide or signal polypeptide.

### Bacterial Plasmids

In some aspects, the rAAV vectors of the present disclosure can be contained within a bacterial plasmid to allow for propagation of the rAAV vector in vitro. Thus, the present disclosure provides bacterial plasmids comprising any of the rAAV vectors described herein. A bacterial plasmid can further comprise an origin of replication sequence. A bacterial plasmid can further comprise an antibiotic resistance gene. A bacterial plasmid can further comprise a prokaryotic promoter.

### Origin of Replication Sequence

In some aspects, an origin of replication sequence can comprise, consist essentially of, or consist of any origin of replication sequence known in the art (e.g., SEQ ID NO: 17). The origin of replication sequence can be a bacterial origin of replication sequence, thereby allowing the rAAV vector comprising said bacterial origin of replication sequence to be produced, propagated and maintained in bacteria, using methods standard in the art.

### Antibiotic Resistance Genes

In some aspects, viral vectors, plasmids, rAAV vectors, or rAAV viral vectors of the disclosure can comprise an antibiotic resistance gene.

In some aspects, an antibiotic resistance gene can comprise, consist essentially of, or consist of any antibiotic resistance genes known in the art. Examples of antibiotic resistance genes known in the art include, but are not limited to kanamycin resistance genes, spectinomycin resistance genes, streptomycin resistance genes, ampicillin resistance genes, carbenicillin resistance genes, bleomycin resistance genes, erythromycin resistance genes, polymyxin B resistance genes, tetracycline resistance genes and chloramphenicol resistance genes.

In some aspects, an antibiotic resistance gene can comprise, consist essentially of, or consist of a kanamycin antibiotic resistance gene. A kanamycin antibiotic resistance gene can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 18.

### Viral Vectors and rAAV Vectors Comprising the SURF1 Transgene

Disclosed herein are viral vectors comprising a transgenic nucleic acid molecule, wherein the transgenic nucleic acid molecule comprises a nucleic acid sequence as set forth in SEQ ID NO: 22, or a sequence at least 90%, 91%, 92%, 93%, 94%, 95%, 96%, 97%, 98%, 99%, or 100% (or any intermediate percentage) identical thereto, operably linked to a promoter sequence and a poly A sequence. In some aspects, the viral vector comprises in the 5′ to 3′ direction a) a promoter sequence comprising the nucleic acid sequence set forth in SEQ ID NO: 14 or SEQ ID NO: 23, or a sequence at least about 95%, 96%, 97%, 98%, 99%, or 100% (or any intermediate percentage) identical thereto; b) a nucleic acid encoding for a SURF1 polypeptide, comprising a nucleic acid sequence as set forth in SEQ ID NO: 22, or a sequence at least about 90%, 91%, 92%, 93%, 94%, 95%, 96%, 97%, 98%, 99%, or 100% (or any intermediate percentage) identical thereto; c) a polyA sequence comprising the nucleic acid sequence set forth in SEQ ID NO: 21 or SEQ ID NO: 24, or a sequence at least about 95%, 96%, 97%, 98%, 99%, or 100% (or any intermediate percentage) identical thereto. In some aspects, disclosed herein are viral vectors comprising in the 5′ to 3′ direction: a) a first AAV inverted terminal repeat (ITR) sequence; b) a promoter sequence comprising the nucleic acid sequence set forth in SEQ ID NO: 14 or SEQ ID NO: 23, or a sequence at least about 95%, 96%, 97%, 98%, 99%, or 100% (or any intermediate percentage) identical thereto; c) a nucleic acid encoding for a SURF1 polypeptide, comprising a nucleic acid sequence as set forth in SEQ ID NO: 22, or a sequence at least about 90%, 91%, 92%, 93%, 94%, 95%, 96%, 97%, 98%, 99%, or 100% (or any intermediate percentage) identical thereto; d) a polyA sequence comprising the nucleic acid sequence set forth in SEQ ID NO: 21, or SEQ ID NO: 24, or a sequence at least about 95%, 96%, 97%, 98%, 99%, or 100% (or any intermediate percentage) identical thereto; (e) a second AAV ITR sequence; wherein the vector is packaged in an AAV9 capsid.

An “AAV virion” or “AAV viral particle” or “AAV viral vector” or “rAAV viral vector” or “AAV vector particle” or “AAV particle” refers to a viral particle composed of at least one AAV capsid protein and an encapsidated polynucleotide rAAV vector. Thus, production of an rAAV viral vector necessarily includes production of an rAAV vector, as such a vector is contained within an rAAV vector.

As used herein, the term “viral capsid” or “capsid” refers to the proteinaceous shell or coat of a viral particle. Capsids function to encapsidate, protect, transport, and release into the host cell a viral genome. Capsids are generally comprised of oligomeric structural subunits of protein (“capsid proteins”). As used herein, the term “encapsidated” means enclosed within a viral capsid. The viral capsid of AAV is composed of a mixture of three viral capsid proteins: VP1, VP2, and VP3. The mixture of VP1, VP2 and VP3 contains 60 monomers that are arranged in a T=1 icosahedral symmetry in a ratio of 1:1:10 (VP1: VP2: VP3) or 1:1:20 (VP1: VP2: VP3) as described in Sonntag F et al., (June 2010). “A viral assembly factor promotes AAV2 capsid formation in the nucleolus”. Proceedings of the National Academy of Sciences of the United States of America. 107 (22): 10220-5, and Rabinowitz J E, Samulski R J (December 2000). “Building a better vector: the manipulation of AAV virions”. Virology. 278 (2): 301-8, each of which is incorporated herein by reference in its entirety.

The present disclosure provides an rAAV viral vector comprising: a) any of the rAAV vectors described herein; and b) an AAV capsid protein.

In some embodiments, disclosed herein is an rAAV viral vector, comprising a nucleic acid sequence as set forth in SEQ ID NO: 24, or a sequence at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% (or any intermediate percentage) identical thereto.

An AAV capsid protein can be any AAV capsid protein known in the art. An AAV capsid protein can be an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein.

In some aspects, present disclosure provides the following embodiments:
1. An rAAV vector, comprising, in the 5′ to 3′ direction
   a) a first AAV ITR sequence;
   b) a promoter sequence;
   c) a transgene nucleic acid molecule, wherein the transgene nucleic acid molecule comprises a nucleic acid sequence encoding for a SURF1 polypeptide;
   d) a polyA sequence; and
   e) a second AAV ITR sequence.
2. The rAAV vector of embodiment 1, wherein the nucleic acid sequence encoding for a SURF1 polypeptide is a codon optimized nucleic acid sequence.
3. The rAAV vector of embodiment 1 or embodiment 2, wherein the SURF1 polypeptide comprises the amino acid sequence of SEQ ID NO: 1 or SEQ ID NO: 2.
4. The rAAV vector of embodiment 3, wherein the SURF1 polypeptide comprises the amino acid sequence of SEQ ID NO: 1.
5. The rAAV vector of embodiment 3, wherein the SURF1 polypeptide comprises the amino acid sequence of SEQ ID NO: 2.
6. The rAAV vector of any one of the preceding embodiments, wherein the codon optimized transgene nucleic acid molecule eliminates a predicted donor splice site.
7. The rAAV vector of any one of the preceding embodiments, wherein the codon optimized transgene nucleic acid molecule has a higher GC content than the wild-type transgene nucleic acid molecule.
8. The rAAV vector of any one of the preceding embodiments, wherein the GC content of the codon optimized transgene nucleic acid molecule is more evenly distributed across the entire nucleic acid sequence as compared to the wild-type transgene nucleic acid molecule.
9. The rAAV vector of any one of the preceding embodiments, wherein the transgene nucleic acid molecule comprises any one of the nucleic acid sequences put forth in SEQ ID NOs: 3-10 or SEQ ID NO: 22.

10. The rAAV vector of embodiment 9, wherein the transgene nucleic acid molecule comprises the nucleic acid sequence of SEQ ID NO: 3.

11. The rAAV vector of embodiment 9, wherein the transgene nucleic acid molecule comprises the nucleic acid sequence of SEQ ID NO: 4.
12. The rAAV vector of embodiment 9, wherein the transgene nucleic acid molecule comprises the nucleic acid sequence of SEQ ID NO: 5.
13. The rAAV vector of embodiment 9, wherein the transgene nucleic acid molecule comprises the nucleic acid sequence of SEQ ID NO: 6.
14. The rAAV vector of embodiment 9, wherein the transgene nucleic acid molecule comprises the nucleic acid sequence of SEQ ID NO: 7.
15. The rAAV vector of embodiment 9, wherein the transgene nucleic acid molecule comprises the nucleic acid sequence of SEQ ID NO: 8.
16. The rAAV vector of embodiment 9, wherein the transgene nucleic acid molecule comprises the nucleic acid sequence of SEQ ID NO: 9.
17. The rAAV vector of embodiment 9, wherein the transgene nucleic acid molecule comprises the nucleic acid sequence of SEQ ID NO: 10.
18. The rAAV vector of any one of the preceding embodiments, wherein the first AAV ITR sequence is an AAV2 ITR sequence.
19. The rAAV vector of any one of the preceding embodiments, wherein the first AAV ITR sequence comprises the nucleic acid sequence of SEQ ID NO: 12.
20. The rAAV vector of any one of the preceding embodiments, wherein the second AAV ITR sequence is a modified AAV2 ITR sequence.
21. The rAAV vector of any one of the preceding embodiments, wherein the second AAV ITR sequence comprises the nucleic acid sequence of SEQ ID NO: 13.
22. The rAAV vector of any one of the preceding embodiments, wherein the promoter sequence comprises a JeT promoter sequence.
23. The rAAV vector of embodiment 22, wherein the JeT promoter sequence comprises the nucleic acid sequence of SEQ ID NO: 14.
24. The rAAV vector of any one of the preceding embodiments, wherein the promoter sequence comprises a MeP229 promoter sequence.
25. The rAAV vector of embodiment 24, wherein the MeP229 promoter sequence comprises the nucleic acid sequence of SEQ ID NO: 15.
26. The rAAV vector of any one of the preceding embodiments, wherein the polyA sequence comprises an SV40pA sequence.
27. The rAAV vector of embodiment 26, wherein the SV40pA sequence comprises the nucleic acid sequence of SEQ ID NO: 16.
28. An rAAV vector of any one of the preceding embodiments, comprising, in the 5′ to 3′ direction
   a) a first AAV ITR sequence comprising the nucleic acid sequence of SEQ ID NO: 12;
   b) a promoter sequence comprising the nucleic acid sequence of SEQ ID NO: 20;
   c) a transgene nucleic acid molecule, wherein the transgene nucleic acid molecule comprises a nucleic acid sequence encoding for a SURF1 polypeptide, wherein the SURF1 polypeptide comprises the amino acid sequence of SEQ ID NO: 1 or SEQ ID NO: 2;
   d) a polyA sequence comprising the nucleic acid sequence of SEQ ID NO: 21 or SEQ ID NO: 24; and
   e) a second AAV ITR sequence comprising the nucleic acid sequence of SEQ ID NO: 13.
29. An rAAV vector of any one of the preceding embodiments, comprising, in the 5′ to 3′ direction
   a) a first AAV ITR sequence comprising the nucleic acid sequence of SEQ ID NO: 12;
   b) a promoter sequence comprising the nucleic acid sequence of SEQ ID NO: 15;
   c) a transgene nucleic acid molecule, wherein the transgene nucleic acid molecule comprises a nucleic acid sequence encoding for a SURF1 polypeptide, wherein the SURF1 polypeptide comprises the amino acid sequence of SEQ ID NO: 1 or SEQ ID NO: 2;
   d) a polyA sequence comprising the nucleic acid sequence of SEQ ID NO: 21 or SEQ ID NO: 24; and
   e) a second AAV ITR sequence comprising the nucleic acid sequence of SEQ ID NO: 13.
30. An rAAV vector of any one of the preceding embodiments, comprising, in the 5′ to 3′ direction
   a) a first AAV ITR sequence comprising the nucleic acid sequence of SEQ ID NO: 12;
   b) a promoter sequence comprising the nucleic acid sequence of SEQ ID NO: 14;
   c) a transgene nucleic acid molecule, wherein the transgene nucleic acid molecule comprises a nucleic acid sequence encoding for a SURF1 polypeptide, wherein the SURF1 polypeptide comprises the amino acid sequence of SEQ ID NO: 1;
   d) a polyA sequence comprising the nucleic acid sequence of SEQ ID NO: 21 or SEQ ID NO: 24; and
   e) a second AAV ITR sequence comprising the nucleic acid sequence of SEQ ID NO: 13.
31. An rAAV vector of any one of the preceding embodiments, comprising, in the 5′ to 3′ direction
   a) a first AAV ITR sequence comprising the nucleic acid sequence of SEQ ID NO: 12:
   b) a promoter sequence comprising the nucleic acid sequence of SEQ ID NO: 15;
   c) a transgene nucleic acid molecule, wherein the transgene nucleic acid molecule comprises a nucleic acid sequence encoding for a SURF1 polypeptide, wherein the SURF1 polypeptide comprises the amino acid sequence of SEQ ID NO: 1;
   d) a polyA sequence comprising the nucleic acid sequence of SEQ ID NO: 16; and
   e) a second AAV ITR sequence comprising the nucleic acid sequence of SEQ ID NO: 13.
32. An rAAV vector of any one of the preceding embodiments, comprising, in the 5′ to 3′ direction
   a) a first AAV ITR sequence comprising the nucleic acid sequence of SEQ ID NO: 12;
   b) a promoter sequence comprising the nucleic acid sequence of SEQ ID NO: 14;
   c) a transgene nucleic acid molecule, wherein the transgene nucleic acid molecule comprises a nucleic acid sequence encoding for a SURF1 polypeptide, wherein the nucleic acid sequence encoding for a SURF1 polypeptide comprises the nucleic acid sequence put forth in any one of SEQ ID NOs: 3-10 or SEQ ID NO: 22;
   d) a polyA sequence comprising the nucleic acid sequence of SEQ ID NO: 16; and
   e) a second AAV ITR sequence comprising the nucleic acid sequence of SEQ ID NO: 13.
33. An rAAV vector of any one of the preceding embodiments, comprising, in the 5′ to 3′ direction
   a) a first AAV ITR sequence comprising the nucleic acid sequence of SEQ ID NO: 12;
   b) a promoter sequence comprising the nucleic acid sequence of SEQ ID NO: 20;
   c) a transgene nucleic acid molecule, wherein the transgene nucleic acid molecule comprises a nucleic acid sequence encoding for a SURF1 polypeptide, wherein the nucleic acid sequence encoding for a SURF1 polypeptide comprises the nucleic acid sequence put forth in any one of SEQ ID NOs: 3-10 or SEQ ID NO: 22
   d) a polyA sequence comprising the nucleic acid sequence of SEQ ID NO: 21 or SEQ ID NO: 24; and
   e) a second AAV ITR sequence comprising the nucleic acid sequence of SEQ ID NO: 13.
34. An rAAV vector of any one of the preceding embodiments, comprising, in the 5′ to 3′ direction
   a) a first AAV ITR sequence comprising the nucleic acid sequence of SEQ ID NO: 12;
   b) a promoter sequence comprising the nucleic acid sequence of SEQ ID NO: 20;
   c) a transgene nucleic acid molecule, wherein the transgene nucleic acid molecule comprises a nucleic acid sequence encoding for a SURF1 polypeptide, wherein the nucleic acid sequence encoding for a SURF1 polypeptide comprises the nucleic acid sequence of SEQ ID NO: 10;
   d) a polyA sequence comprising the nucleic acid sequence of SEQ ID NO: 21, or SEQ ID NO: 24; and
   e) a second AAV ITR sequence comprising the nucleic acid sequence of SEQ ID NO: 13.
35. An rAAV vector of any one of the preceding embodiments, comprising, in the 5′ to 3′ direction
   a) a first AAV ITR sequence comprising the nucleic acid sequence of SEQ ID NO: 12;
   b) a promoter sequence comprising the nucleic acid sequence of SEQ ID NO: 20;
   c) a transgene nucleic acid molecule, wherein the transgene nucleic acid molecule comprises a nucleic acid sequence encoding for a SURF1 polypeptide, wherein the nucleic acid sequence encoding for a SURF1 polypeptide comprises the nucleic acid sequence of SEQ ID NO: 6;
   d) a polyA sequence comprising the nucleic acid sequence of SEQ ID NO: 21, or SEQ ID NO: 24; and
   e) a second AAV ITR sequence comprising the nucleic acid sequence of SEQ ID NO: 13.
36. An rAAV vector of any one of the preceding embodiments, comprising, in the 5′ to 3′ direction
   a) a first AAV ITR sequence comprising the nucleic acid sequence of SEQ ID NO: 12:
   b) a promoter sequence comprising the nucleic acid sequence of SEQ ID NO: 20;
   c) a transgene nucleic acid molecule, wherein the transgene nucleic acid molecule comprises a nucleic acid sequence encoding for a SURF1 polypeptide, wherein the nucleic acid sequence encoding for a SURF1 polypeptide comprises the nucleic acid sequence of SEQ ID NO: 5;
   d) a polyA sequence comprising the nucleic acid sequence of SEQ ID NO: 21, or SEQ ID NO: 24; and
   e) a second AAV ITR sequence comprising the nucleic acid sequence of SEQ ID NO: 13.
37. An rAAV vector of any one of the preceding embodiments, comprising, in the 5′ to 3′ direction
   a) a first AAV ITR sequence comprising the nucleic acid sequence of SEQ ID NO: 12;
   b) a promoter sequence comprising the nucleic acid sequence of SEQ ID NO: 20;
   c) a transgene nucleic acid molecule, wherein the transgene nucleic acid molecule comprises a nucleic acid sequence encoding for a SURF1 polypeptide, wherein the nucleic acid sequence encoding for a SURF1 polypeptide comprises the nucleic acid sequence of SEQ ID NO: 5;
   d) a polyA sequence comprising the nucleic acid sequence of SEQ ID NO: 16; and
   e) a second AAV ITR sequence comprising the nucleic acid sequence of SEQ ID NO: 13.
38. An rAAV vector of any one of the preceding embodiments, comprising, in the 5′ to 3′ direction
   a) a first AAV ITR sequence comprising the nucleic acid sequence of SEQ ID NO: 12;
   b) a promoter sequence comprising the nucleic acid sequence of SEQ ID NO: 20;
   c) a transgene nucleic acid molecule, wherein the transgene nucleic acid molecule comprises a nucleic acid sequence encoding for a SURF1 polypeptide, wherein the nucleic acid sequence encoding for a SURF1 polypeptide comprises the nucleic acid sequence of SEQ ID NO: 3;
   d) a polyA sequence comprising the nucleic acid sequence of SEQ ID NO: 21, and SEQ ID NO: 24; and
   e) a second AAV ITR sequence comprising the nucleic acid sequence of SEQ ID NO: 13.
39. An rAAV vector of any one of the preceding embodiments, comprising, in the 5′ to 3′ direction
   a) a first AAV ITR sequence comprising the nucleic acid sequence of SEQ ID NO: 20;
   b) a promoter sequence comprising the nucleic acid sequence of SEQ ID NO: 15;
   c) a transgene nucleic acid molecule, wherein the transgene nucleic acid molecule comprises a nucleic acid sequence encoding for a SURF1 polypeptide, wherein the nucleic acid sequence encoding for a SURF1 polypeptide comprises the nucleic acid sequence of SEQ ID NO: 10;
   d) a polyA sequence comprising the nucleic acid sequence of SEQ ID NO: 16; and
   e) a second AAV ITR sequence comprising the nucleic acid sequence of SEQ ID NO: 21, or SEQ ID NO: 24.
41. An rAAV viral vector comprising:
   a) an rAAV vector of any one of the preceding embodiments; and
   b) an AAV capsid protein.
42. The rAAV viral vector of embodiment 44, wherein the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein.
43. The rAAV viral vector of embodiment 45, wherein the AAV capsid protein is an AAV1 capsid protein.
44. The rAAV viral vector of embodiment 45, wherein the AAV capsid protein is an AAV2 capsid protein.
45. The rAAV viral vector of embodiment 45, wherein the AAV capsid protein is an AAV3 capsid protein.
46. The rAAV viral vector of embodiment 45, wherein the AAV capsid protein is an AAV4 capsid protein.
47. The rAAV viral vector of embodiment 45, wherein the AAV capsid protein is an AAV5 capsid protein.
48. The rAAV viral vector of embodiment 45, wherein the AAV capsid protein is an AAV6 capsid protein.
49. The rAAV viral vector of embodiment 45, wherein the AAV capsid protein is an AAV7 capsid protein.
50. The rAAV viral vector of embodiment 45, wherein the AAV capsid protein is an AAV8 capsid protein.
51. The rAAV viral vector of embodiment 45, wherein the AAV capsid protein is an AAV9 capsid protein.
52. The rAAV viral vector of embodiment 45, wherein the AAV capsid protein is an AAV10 capsid protein.
53. The rAAV viral vector of embodiment 45, wherein the AAV capsid protein is an AAV11 capsid protein.
54. The rAAV viral vector of embodiment 45, wherein the AAV capsid protein is an AAV12 capsid protein.
55. The rAAV viral vector of embodiment 45, wherein the AAV capsid protein is an AAV13 capsid protein.
56. The rAAV viral vector of embodiment 45, wherein the AAV capsid protein is an AAVPHP.B capsid protein.
57. The rAAV viral vector of embodiment 45, wherein the AAV capsid protein is an AAVrh74 capsid protein.
58. The rAAV viral vector of embodiment 45, wherein the AAV capsid protein is an AAVrh.10 capsid protein.
59. The rAAV vector of embodiment 9, wherein the transgene nucleic acid molecule comprises the nucleic acid sequence of SEQ ID NO: 22
60. A rAAV vector, comprising in the 5′ to 3′ direction:
   a) a promoter sequence comprising the nucleic acid sequence set forth in SEQ ID NO: 14 or SEQ ID NO: 23, or a sequence at least about 95% identical thereto;
   b) a transgene nucleic acid molecule, wherein the transgene nucleic acid molecule comprises a nucleic acid encoding for a SURF1 polypeptide, comprising a nucleic acid sequence as set forth in SEQ ID NO: 22, or a sequence at least about 90% identical thereto;
   c) a polyA sequence comprising the nucleic acid sequence set forth in SEQ ID NO: 21, or a sequence at least about 95% identical thereto.
61. The rAAV vector of embodiment 60, comprising in the 5′ to 3′ direction:
   a) a first AAV inverted terminal repeat (ITR) sequence;
   b) a promoter sequence comprising the nucleic acid sequence set forth in SEQ ID NO: 14 or SEQ ID NO: 23, or a sequence at least about 95% identical thereto;
   c) a transgene nucleic acid molecule, wherein the transgene nucleic acid molecule comprises a nucleic acid encoding for a SURF1 polypeptide, comprising a nucleic acid sequence as set forth in SEQ ID NO: 22, or a sequence at least about 90% identical thereto;
   d) a polyA sequence comprising the nucleic acid sequence set forth in SEQ ID NO: 21 or SEQ ID NO: 24, or a sequence at least about 95% identical thereto;
   (e) a second AAV ITR sequence;
      wherein the vector is packaged in an AAV9 capsid.
62. The rAAV of embodiment 61, comprising a nucleic acid sequence as set forth in SEQ ID NO: 24, or a sequence at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% (or any intermediate percentage) identical thereto.

### Compositions and Pharmaceutical Compositions

The present disclosure provides compositions comprising any of the isolated polynucleotides, rAAV, vectors and/or rAAV viral vectors described herein. In some aspects, the compositions can be pharmaceutical compositions. Accordingly, the present disclosure provides pharmaceutical compositions comprising any of the isolated polynucleotides, rAAV vectors, and/or rAAV viral vectors described herein.

The pharmaceutical composition, as described herein, may be formulated by any methods known or developed in the art of pharmacology, which include but are not limited to contacting the active ingredients (e.g., viral particles or recombinant vectors) with an excipient and/or additive or other accessory ingredient, dividing or packaging the product to a dose unit. The viral particles of this disclosure may be formulated with desirable features, e.g., increased stability, increased cell transfection, sustained or delayed release, biodistributions or tropisms, modulated or enhanced translation of encoded protein in vivo, and the release profile of encoded protein in vivo.

As such, the pharmaceutical composition may further comprise saline, lipidoids, liposomes, lipid nanoparticles, polymers, lipoplexes, core-shell nanoparticles, peptides, proteins, cells transfected with viral vectors (e.g., for transplantation into a subject), nanoparticle mimics or combinations thereof. In some aspects, the pharmaceutical composition is formulated as a nanoparticle. In some aspects, the nanoparticle is a self-assembled nucleic acid nanoparticle.

A pharmaceutical composition in accordance with the present disclosure may be prepared, packaged, and/or sold in bulk, as a single unit dose, and/or as a plurality of single unit doses. The amount of the active ingredient is generally equal to the dosage of the active ingredient which would be administered to a subject and/or a convenient fraction of such a dosage such as, for example, one-half or one-third of such a dosage. The formulations of the invention can include one or more excipients and/or additives, each in an amount that together increases the stability of the viral vector, increases cell transfection or transduction by the viral vector, increases the expression of viral vector encoded protein, and/or alters the release profile of viral vector encoded proteins. In some aspects, the pharmaceutical composition comprises an excipient and/or additives. Non limiting examples of excipients and/or additives include solvents, dispersion media, diluents, or other liquid vehicles, dispersion or suspension aids, surface active agents, isotonic agents, thickening or emulsifying agents, preservatives, or combination thereof.

In some aspects, the pharmaceutical composition comprises a cryoprotectant. The term “cryoprotectant” refers to an agent capable of reducing or eliminating damage to a substance during freezing. Non-limiting examples of cryoprotectants include sucrose, trehalose, lactose, glycerol, dextrose, raffinose and/or mannitol.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

In some aspects, a pharmaceutical composition of the present disclosure can comprise phosphate-buffered saline, D-sorbitol, sodium chloride, pluronic F-68 or any combination thereof.

In some aspects, a pharmaceutical composition can comprise sodium chloride, wherein the sodium chloride is present at a concentration of about 100 mM to about 500 mM, or about 200 mM to about 400 mM, or about 300 mM to about 400 mM. In some aspects, the sodium chloride can be present at a concentration of about 350 mM.

In some aspects, a pharmaceutical composition can comprise D-sorbitol, wherein the D-sorbitol is present at a concentration of about 1% to about 10%, or about 2.5% to about 7.5%. In some aspects, the D-sorbitol can be present at a concentration of about 5%.

In some aspects, a pharmaceutical composition can comprise pluronic F-68, wherein the pluronic F-68 is present at a concentration of about 0.00001% to about 0.01%, or about 0.0005% to about 0.005%. In some aspects, the pluronic F-68 can be present at a concentration of about 0.001%.

Thus, the present disclosure provides a pharmaceutical composition comprising an rAAV vector and/or rAAV viral vector of the present disclosure in a phosphate-buffered saline solution, wherein the pharmaceutical composition further comprises sodium chloride at a concentration of 350 mM, D-sorbitol at a concentration of 5% and pluronic F-68 at a concentration of 0.001%.

Thus, the present disclosure provides a pharmaceutical composition comprising an rAAV vector and/or rAAV viral vector of the present disclosure, wherein the pharmaceutical composition further comprises sodium chloride at a concentration of 350 mM, D-sorbitol at a concentration of 5% and pluronic F-68 at a concentration of 0.001%.

Thus, the present disclosure provides a pharmaceutical composition comprising an rAAV vector and/or rAAV viral vector of the present disclosure in a phosphate-buffered saline solution, wherein the pharmaceutical composition further comprises sodium chloride at a concentration of 350 mM, D-sorbitol at a concentration of 5%.

Thus, the present disclosure provides a pharmaceutical composition comprising an rAAV vector and/or rAAV viral vector of the present disclosure, wherein the pharmaceutical composition further comprises sodium chloride at a concentration of 350 mM, D-sorbitol at a concentration of 5%.

### Methods of Using the Compositions of the Disclosure

The present disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. Preferably, the subject is human. The terms “subject” and “patient” are used interchangeably herein.

This disclosure provides methods of preventing or treating a disorder, comprising, consisting essentially of, or consisting of administering to a subject a therapeutically effective amount of any one of the pharmaceutical compositions disclosed herein.

In some aspects, the disease can be a genetic disorder involving a SURF1 gene.

In some aspects, the disclosure provides methods of preventing or treating SURF1 deficiency, Leigh Syndrome, Mitochondrial complex IV deficiency or Charcot-Marie-Tooth disease 4K.

In some aspects, a disease can be a disease that is characterized by the loss-of-function of at least one copy of the SURF1 gene in the genome of a subject. In some aspects, a disease can be a disease that is characterized by a decrease in function of at least one copy of a SURF1 gene in the genome of a subject. In some aspects, a disease can be a disease that is characterized by at least one mutation in at least one mutation in at least one copy of a SURF1 gene in the genome of the subject.

A mutation in a SURF1 gene can be any type of mutation that is known in the art. Non-limiting examples of mutations include somatic mutations, single nucleotide variants (SNVs), nonsense mutations, insertions, deletions, duplications, frameshift mutations, repeat expansions, short insertions and deletions (INDELs), long INDELs, alternative splicing, the products of alternative splicing, altered initiation of translation, the products of altered initiation of translation, proteomic cleavage, and the products of proteomic cleavage.

In some aspects, a disease can be a disease that is characterized by a decrease in expression of a SURF1 gene in a subject as compared to a control subject that does not have the disease. In some aspects, the decrease in expression can be at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 100%.

In some aspects, a disease can be a disease that is characterized by a decrease in the amount of SURF1 in a subject as compared to a control subject that does not have the disease. In some aspects, the decrease in the amount of SURF1 can be at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 100%.

In some aspects, a disease can be a disease that is characterized by a decrease in the activity of SURF1 in a subject as compared to a control subject that does not have the disease. In some aspects, the decrease in the activity of SURF1 can be at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 100%.

A subject to be treated using the methods, compositions, pharmaceutical compositions, rAAV vectors or rAAV viral vectors of the present disclosure can have any of the diseases and/or symptoms described herein.

In some aspects, a subject can be less than 0.5 years of age, or less than 1 year of age, or less than 1.5 years of age, or less than 2 years of age, or at less than 2.5 years of age, or less than 3 years of age, or less than 3.5 years of age, or less than 3.5 years of age, or less than 4 years of age, or less than 4.5 years of age, or less than 5 years of age, or less than 5.5 years of age, or less than 6 years of age, or less than 6.5 years of age, or less than 7 years of age, or less than 7.5 years of age, or less than 8 years of age, or less than 8.5 years of age, or less than 9 years of age, or less than 9.5 years of age, or less than 10 years of age. In some aspects the subject can be less than 11 years of age, less than 12 years of age, less than 13 years of age, less than 14 years of age, less than 15 years of age, less than 20 years of age, less than 30 years of age, less than 40 years of age, less than 50 years of age, less than 60 years of age, less than 70 years of age, less than 80 years of age, less than 90 years of age, less than 100 years of age, less than 110 years of age, or less than 120 years of age. In some aspects, a subject can be less than 0.5 years of age. In some aspects, a subject can be less than 4 years of age. In some aspects, a subject can be less than 10 years of age.

The methods of treatment and prevention disclosed herein may be combined with appropriate diagnostic techniques to identify and select patients for the therapy or prevention.

The disclosure provides methods of increasing the level of a protein in a host cell, comprising contacting the host cell with any one of the rAAV viral vectors disclosed herein, wherein the rAAV viral vectors comprises any one of the rAAV vectors disclosed herein, comprising a transgene nucleic acid molecule encoding the protein. In some aspects, the protein is a therapeutic protein. In some aspects, the host cell is in vitro, in vivo, or ex vivo. In some aspects, the host cell is derived from a subject. In some aspects, the subject suffers from a disorder, which results in a reduced level and/or functionality of the protein, as compared to the level and/or functionality of the protein in a normal subject.

In some aspects, the level of the protein is increased to level of about 1×10⁻⁷ ng, about 3×10⁻⁷ ng, about 5×10⁻⁷ ng, about 7×10⁻⁷ ng, about 9×10⁻⁷ ng, about 1×10⁻⁶ ng, about 2×10⁻⁶ ng, about 3×10⁻⁶ ng, about 4×10⁻⁶ ng, about 6×10⁻⁶ ng, about 7×10⁻⁶ ng, about 8×10⁻⁶ ng, about 9×10⁻⁶ ng, about 10×10⁻⁶ ng, about 12×10⁻⁶ ng, about 14×10⁻⁶ ng, about 16×10⁻⁶ ng, about 18×10⁻⁶ ng, about 20×10⁻⁶ ng, about 25×10⁻⁶ ng, about 30×10⁻⁶ ng, about 35×10⁻⁶ ng, about 40×10⁻⁶ ng, about 45×10⁻⁶ ng, about 50×10⁻⁶ ng, about 55×10⁻⁶ ng, about 60×10⁻⁶ ng, about 65×10⁻⁶ ng, about 70×10⁻⁶ ng, about 75×10⁻⁶ ng, about 80×10⁻⁶ ng, about 85×10⁻⁶ ng, about 90×10⁻⁶ ng, about 95×10⁻⁶ ng, about 10×10⁻⁵ ng, about 20×10⁻⁵ ng, about 30×10⁻⁵ ng, about 40×10⁻⁵ ng, about 50×10⁻⁵ ng, about 60×10⁻⁵ ng, about 70×10⁻⁵ ng, about 80×10⁻⁵ ng, or about 90×10⁻⁵ ng in the host cell.

The disclosure provides methods of introducing a gene of interest to a cell in a subject comprising contacting the cell with an effective amount of any one of the rAAV viral vectors disclosed herein, wherein the rAAV viral vectors contain any one of the rAAV vectors disclosed herein, comprising the gene of interest.

In some aspects of the methods of the present disclosure, a subject can also be administered a prophylactic immunosuppressant treatment regimen in addition to being administered an rAAV vector or rAAV viral vector of the present disclosure. In some aspects, an immunosuppressant treatment regimen can comprise administering at least one immunosuppressive therapeutic. Non limiting examples of immunosuppressive therapeutics include, but are not limited to, Sirolimus (rapamycin), acetaminophen, diphenhydramine, IV methylprednisolone, prednisone, or any combination thereof. An immunosuppressive therapeutic can be administered prior to the day of administration of the rAAV vector and/or rAAV viral vector, on the same day as the administration of the rAAV vector and/or rAAV viral vector, or any day following the administration of the rAAV vector and/or rAAV viral vector.

A “subject” of diagnosis or treatment is a cell or an animal such as a mammal, or a human. A subject is not limited to a specific species and includes non-human animals subject to diagnosis or treatment and those subject to infections or animal models, including, without limitation, simian, murine, rat, canine, or leporid species, as well as other livestock, sport animals, or pets. In some aspects, the subject is a human.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein the term “effective amount” intends to mean a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of gene therapy, the effective amount can be the amount sufficient to result in regaining part or full function of a gene that is deficient in a subject. In some aspects, the effective amount of an rAAV viral vector is the amount sufficient to result in expression of a gene in a subject such that SURF1 is produced. In some aspects, the effective amount is the amount required to increase galactose metabolism in a subject in need thereof. The skilled artisan will be able to determine appropriate amounts depending on these and other factors.

In some aspects, the effective amount will depend on the size and nature of the application in question. It will also depend on the nature and sensitivity of the target subject and the methods in use. The skilled artisan will be able to determine the effective amount based on these and other considerations. The effective amount may comprise, consist essentially of, or consist of one or more administrations of a composition depending on the embodiment.

As used herein, the term “administer” or “administration” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and other animals, treating veterinarian.

Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art. Non-limiting examples of such suitable dosages may be as low as 10⁹ vector genomes to as much as 10₁₇ vector genomes per administration.

In some aspects of the methods described herein, the number of vector particles (e.g., rAAV viral vectors) administered to the subject ranges from about 10₉ to about 10₁₇. In some aspects, about 10₁₀ to about 10₁₂, about 10₁₁ to about 10₁₃, about 10₁₁ to about 10₁₂, about 10₁₁ to about 10₁₄, about 10₁₂ to about 10₁₆, about 10₁₃ to about 10₁₆, about 10₁₄ to about 10₁₅, about 5×10¹¹ to about 5×10₁₂, or about 10₁₂ to about 10₁₃ viral particles are administered to the subject.

In some aspects of the methods described herein, the number of viral particles (e.g., rAAV viral vectors) administered to the subject is at least about 10₁₀, or at least about 10₁₁, or at least about 10₁₂, or at least about 10₁₃, or at least about 10₁₄, or at least about 10₁₅, or at least about 10₁₆, or at least about 10₁₇ viral particles.

In some aspects of the methods described herein, the number of viral particles (e.g., rAAV viral vectors) administered to the subject can depend on the age of the subject. In non-limiting examples, a subject that is 7 years of age or older can be administered about 10×10¹⁴ viral particles, a subject that is about 4 years of age to about 7 years of age can be administered about 10×10¹⁴ viral particles, a subject that is about 3 years of age to about 4 years of age can be administered about 9×10¹⁴ viral particles, a subject that is about 2 years of age to about 3 years of age can be about 8.2×10¹⁴ viral particles, a subject that is about 1 year of age to about 2 years of age can be administered about 7.3×10¹⁴ viral particles, a subject that is about 0.5 years of age to about 1 year of age can be administered about 4×10¹⁴ viral particles, or a subject that is less than 0.5 years of age can be administered 3×10¹⁴ viral particles.

In some aspects, the amounts of viral particles in a composition, pharmaceutical composition, or the amount of viral particles administered to a patient can calculated based on the percentage of viral particles that are predicted to contain viral genomes.

In some aspects, rAAV viral vectors of the present disclosure can be introduced to the subject intravenously, intrathecally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally; such introduction may also be intra-arterial, intracardiac, subventricular, epidural, intracerebral, intracerebroventricular, sub-retinal, intravitreal, intraarticular, intraperitoneal, intrauterine, or any combination thereof. In some aspects, the viral particles are delivered to a desired target tissue, e.g., to the lung, eye, or CNS, as non-limiting examples. In some aspects, delivery of viral particles is systemic. The intracisternal route of administration involves administration of a drug directly into the cerebrospinal fluid of the brain ventricles. It could be performed by direct injection into the cisterna magna or via a permanently positioned tube. In some aspects, the rAAV viral vectors of the present disclosure are administered intrathecally.

In some aspects, the rAAV viral vectors of the present disclosure repair a gene deficiency in a subject. In some aspects, the ratio of repaired target polynucleotide or polypeptide to unrepaired target polynucleotide or polypeptide in a successfully treated cell, tissue, organ or subject is at least about 1.5:1, about 2:1, about 3:1, about 4:1, about 5:1, about 6:1, about 7:1, about 8:1, about 9:1, about 10:1, about 20:1, about 50:1, about 100:1, about 1000:1, about 10,000:1, about 100,000:1, or about 1,000,000:1. The amount or ratio of repaired target polynucleotide or polypeptide can be determined by any method known in the art, including but not limited to western blot, northern blot, Southern blot, PCR, sequencing, mass spectrometry, flow cytometry, immunohistochemistry, immunofluorescence, fluorescence in situ hybridization, next generation sequencing, immunoblot, and ELISA.

Administration of the rAAV vectors, rAAV viral vectors, compositions or pharmaceutical compositions of this disclosure can be effected in one dose, continuously or intermittently throughout the course of treatment. In some aspects, the rAAV vectors, rAAV viral vectors, compositions, or pharmaceutical compositions of this disclosure are parenterally administered by injection, infusion, or implantation.

In some aspects, the rAAV viral vectors of this disclosure show enhanced tropism for brain and cervical spine. In some aspects, the rAAV viral vectors of the disclosure can cross the blood-brain-barrier (BBB).

### Methods of Manufacture

A variety of approaches may be used to produce rAAV viral vectors of the present disclosure. In some aspects, packaging is achieved by using a helper virus or helper plasmid and a cell line. The helper virus or helper plasmid contains elements and sequences that facilitate viral vector production. In another aspect, the helper plasmid is stably incorporated into the genome of a packaging cell line, such that the packaging cell line does not require additional transfection with a helper plasmid.

In some aspects, the cell is a packaging or helper cell line. In some aspects, the helper cell line is eukaryotic cell; for example, an HEK 293 cell or 293T cell. In some aspects, the helper cell is a yeast cell or an insect cell.

In some aspects, the cell comprises a nucleic acid encoding a tetracycline activator protein; and a promoter that regulates expression of the tetracycline activator protein. In some aspects, the promoter that regulates expression of the tetracycline activator protein is a constitutive promoter. In some aspects, the promoter is a phosphoglycerate kinase promoter (PGK) or a CMV promoter.

A helper plasmid may comprise, for example, at least one viral helper DNA sequence derived from a replication-incompetent viral genome encoding in trans all virion proteins required to package a replication incompetent AAV, and for producing virion proteins capable of packaging the replication-incompetent AAV at high titer, without the production of replication-competent AAV.

Helper plasmids for packaging AAV are known in the art, see, e.g., U.S. Patent Pub. No. 2004/0235174 A1, incorporated herein by reference. As stated therein, an AAV helper plasmid may contain as helper virus DNA sequences, by way of non-limiting example, the Ad5 genes E2A, E4 and VA, controlled by their respective original promoters or by heterologous promoters. AAV helper plasmids may additionally contain an expression cassette for the expression of a marker protein such as a fluorescent protein to permit the simple detection of transfection of a desired target cell.

The disclosure provides methods of producing rAAV viral vectors comprising transfecting a packaging cell line with any one of the AAV helper plasmids disclosed herein; and any one of the rAAV vectors disclosed herein. In some aspects, the AAV helper plasmid and rAAV vector are co-transfected into the packaging cell line. In some aspects, the cell line is a mammalian cell line, for example, human embryonic kidney (HEK) 293 cell line. The disclosure provides cells comprising any one of the rAAV vectors and/or rAAV viral vectors disclosed herein.

As used herein, the term “helper” in reference to a virus or plasmid refers to a virus or plasmid used to provide the additional components necessary for replication and packaging of any one of the rAAV vectors disclosed herein. The components encoded by a helper virus may include any genes required for virion assembly, encapsidation, genome replication, and/or packaging. For example, the helper virus or plasmid may encode necessary enzymes for the replication of the viral genome. Non-limiting examples of helper viruses and plasmids suitable for use with AAV constructs include pHELP (plasmid), adenovirus (virus), or herpesvirus (virus). In some aspects, the pHELP plasmid may be the pHELPK plasmid, wherein the ampicillin expression cassette is exchanged with a kanamycin expression cassette.

As used herein, a packaging cell (or a helper cell) is a cell used to produce viral vectors. Producing recombinant AAV viral vectors requires Rep and Cap proteins provided in trans as well as gene sequences from Adenovirus that help AAV replicate. In some aspects, Packaging/helper cells contain a plasmid is stably incorporated into the genome of the cell. In other aspects, the packaging cell may be transiently transfected. Typically, a packaging cell is a eukaryotic cell, such as a mammalian cell or an insect cell.

### Kits

The isolated polynucleotides, rAAV vectors, rAAV viral vectors, compositions, and/or pharmaceutical compositions described herein may be assembled into pharmaceutical or diagnostic or research kits to facilitate their use in therapeutic, diagnostic, or research applications. In some aspects, the kits of the present disclosure include any one of the isolated polynucleotides, rAAV vectors, rAAV viral vectors, compositions, pharmaceutical compositions, host cells, isolated tissues, as described herein.

In some aspects, a kit further comprises instructions for use. Specifically, such kits may include one or more agents described herein, along with instructions describing the intended application and the proper use of these agents. In some aspects, the kit may include instructions for mixing one or more components of the kit and/or isolating and mixing a sample and applying to a subject. In some aspects, agents in a kit are in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. In some aspects, the compositions may be provided in a preservation solution (e.g., cryopreservation solution). Non-limiting examples of preservation solutions include DMSO, paraformaldehyde, and CryoStor® (Stem Cell Technologies, Vancouver, Canada). In some aspects, the preservation solution contains an amount of metalloprotease inhibitors.

In some aspects, the kit contains any one or more of the components described herein in one or more containers. Thus, in some aspects, the kit may include a container housing agents described herein. The agents may be in the form of a liquid, gel or solid (powder). The agents may be prepared sterilely, packaged in a syringe and shipped refrigerated. Alternatively, they may be housed in a vial or other container for storage. A second container may have other agents prepared sterilely. Alternatively, the kit may include the active agents premixed and shipped in a syringe, vial, tube, or other container. The kit may have one or more or all of the components required to administer the agents to a subject, such as a syringe, topical application devices, or IV needle tubing and bag.

### EXAMPLES

### Example 1: Codon Optimization of a SURF1 Transgene

A transgene nucleic acid molecule comprising a codon optimized nucleic acid sequence encoding a SURF1 polypeptide was designed. The SURF1 transgene sequence was codon optimized to facilitate the ease of transgene detection by molecular methods, and minimize rare unrelated transcripts or aberrant splicing variants. The SUR1 transgene sequence was codon optimized to remove rare codons, cryptic splice sites, and cryptic start sites with an altered nucleic acid sequence that still encodes the fully WT protein. The purpose of the codon optimization was to enable easier tracking of the vector sequence to distinguish it from the endogenous chromosomal gene sequence. Molecular methods such as polymerase chain reaction, Southern blot, Northern blot, in situ hybridization, etc. can thus be used to readily detect the distribution of the vector transgene and expressed transgene mRNA in cells, tissues, or body fluids. These changes may also increase expression, and may also reduce expression of alternative proteins that could be immunogenic or otherwise detrimental compared to the natural unmodified gene sequence.

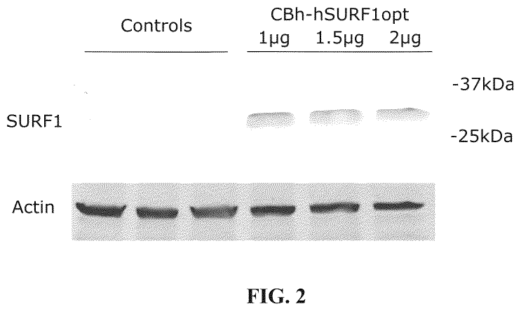

The expression of hSUR1opt is controlled by hybrid chicken beta actin (CBA) promoter (CBh) and BGH poly (A) tail (AAV9/SURF1). The CBh promoter was used to ensure universal expression of hSUR1opt, which mimic the characteristics of endogenous SURF1. The ability of the hSUR1opt transgene to express hSURF1 protein was demonstrated by transfecting the plasmid into HEK293 cells, as shown in FIG. 2.

### Example 2: Intrathecal (IT) Delivery of AAV9/hSURF1opt Results in SURF1 Expression in Various Brain Regions and Spinal Cord

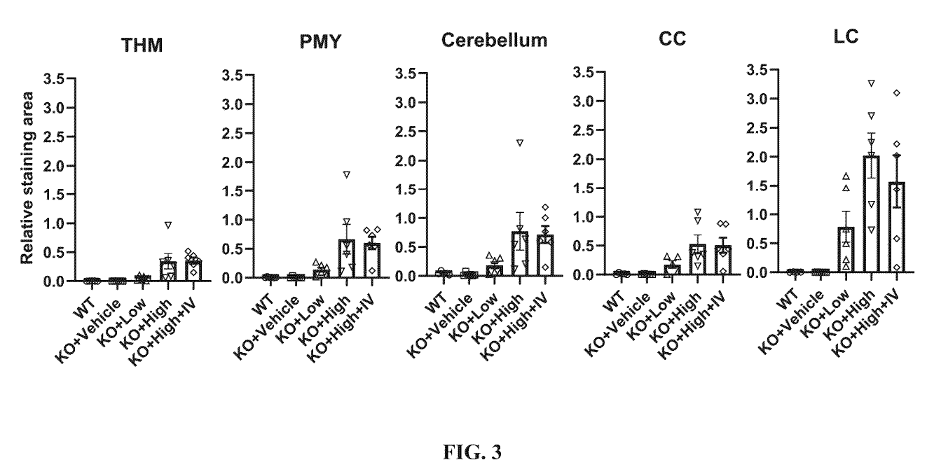

RNAscope was used to specifically detect mRNA of the hSUR1opt transgene in AAV9/hSURF1-treated mice. Brain and spinal cord were collected from SURF1 KO mice 4-weeks post AAV9/hSURF1 treatment. The expression level of hSUR1opt mRNA in each experimental group was analyzed and is shown in FIG. 3. Percent area staining positive for hSURF1opt mRNA by tissue region (n=6 per group). THM: thalamus, hypothalamus, and midbrain; PMY: pon and medulla; CC: cervical spinal cord; LC: lumbar spinal cord, Low: low IT dose, High: high IT dose, High+IV: combination intrathecal and intravenous dose. Each data point represents measurement from an individual animal, with bars representing the mean±SEM.

The results show that SURF1 mRNA was successfully expressed in all disease-relevant brain areas, cervical spinal cord (furthest from the injection site) and lumbar spinal cord (closest to the injection site). The IT+IV combo treatment did not show significant improvement compared with IT only treatment group.

### Example 3: Intrathecal Delivery of AAV9/hSURF1opt Rescues COX Deficiency

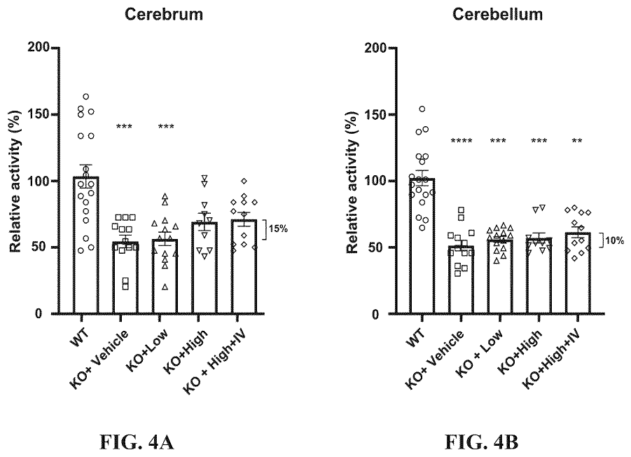

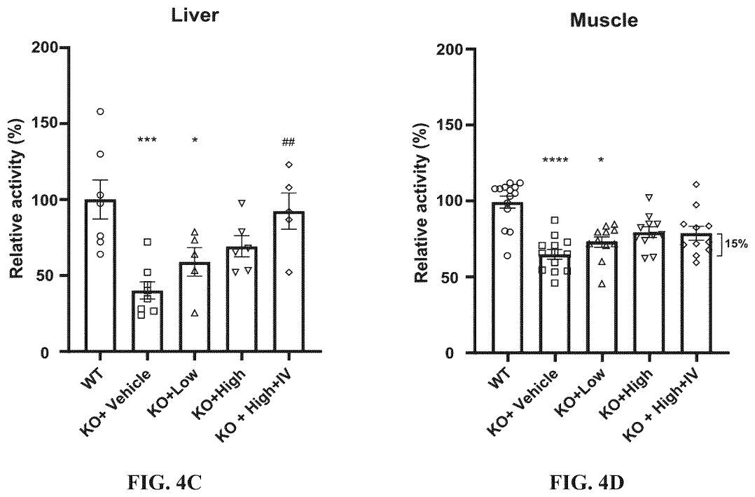

Cytochrome C Oxidase (COX) activity was measured in brain (cerebrum and cerebellum), liver and muscle of AAV9/hSURF1opt treated SURF1 KO mice, and cerebellum and cerebrum were tested separately. As shown in FIG. 4, COX activity of SURF1 KO mice was reduced between 30-60% in all tissues tested compared with that of WT mice. FIG. 4A depicts COX activity of cerebrum (n=10-18 per group), FIG. 4B depicts cerebellum (n=10-18 per group), FIG. 4C depicts liver (n=5-8 per group), and FIG. 4D depicts muscle (n=10-14 per group) of WT and SURF1 KO mice with assigned treatments. All data were normalized to the average of WT mice. Each data point represents measurement from an individual animal, with bars representing the mean±SEM. *p<0.05, **p<0.01, ***p<0.001, *p<0.0001 compared with WT mice. ##p<0.01 compared with KO+Vehicle mice.

AAV9/hSURF1 administered through IT partially but significantly restored COX activity, and the effect increased with dose escalation.

### Example 4: Intrathecal Delivery of AAV9/hSURF1 Ablated Exhaustive Exercise-Induced Lactic Acidosis in Aged SURF1 KO Mice

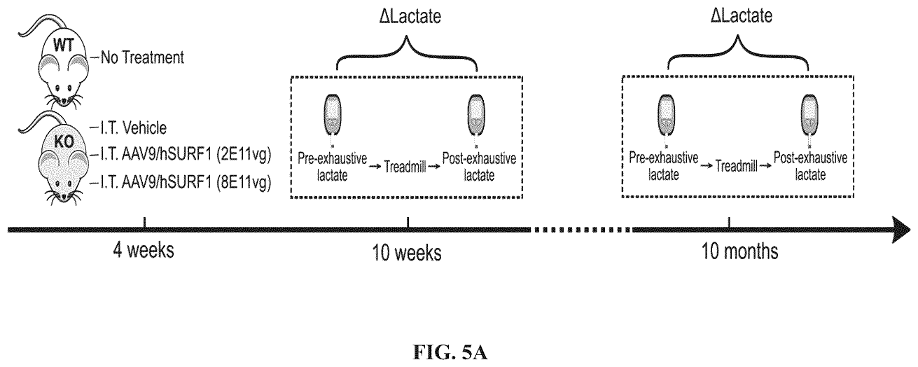

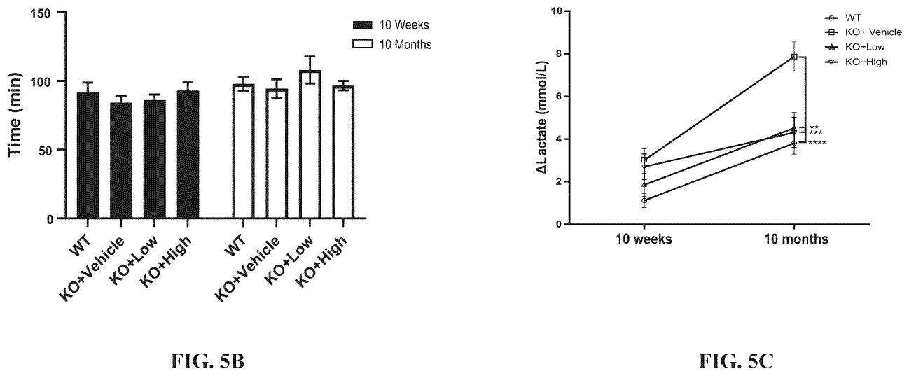

Serum lactate is increased in SURF1 KO mice and human patients, which is associated with mitochondrial dysfunction. The impact of AAV9/hSURF1 treatment on serum lactate levels was assessed in SURF1 KO mice. The study scheme was shown in FIG. 5A. The mice were tested at two ages, 10 weeks, which is 6 weeks after AAV treatment, and 10 months. SURF1 KO mice do not show lactic acidosis under resting state, but their serum lactate was significantly increased after exhaustive exercise. Thus, the change of lactate (ΔLactate) was measured after exhaustion from treadmill running. As shown in FIG. 5B, there was no differences in their running time among groups at both 10-weeks-old and 10-months-old, suggesting SURF1 KO mice were able to maintain normal endurance capacity even with deficient COX. However, as shown in FIG. 5C, their lactate was increased after the exercise at both ages, while the increment at 10 months old was more significant. FIG. 5B depicts running time on treadmill until exhaustion. FIG. 5C depicts ΔLactate (post-exhaustion lactate-pre-exhaustion lactate) of mice from all tested groups at 10 weeks and 10 months old. Data shown as mean+SEM. **p<0.01, ***p<0.001, and ****p<0.0001 analyzed using two-way ANOVA with Sidak's multiple comparisons test. #p<0.05, ###p<0.001 analyzed using paired t-test.

Low and high dose AAV9/hSURF1 treatment ablated the exhaustive exercise-induced lactic acidosis, suggesting that the gene replacement therapy significantly improved the mitochondrial metabolic functions.

The following examples pertain to versions of the hSURF1opt (hSURF1v1: SEQ ID NO: 10) referenced herein above or further optimized version hSURF1v2 (SEQ ID NO: 22).

### Example 5: AAV9/hSURF1v1 Induced Cytotoxicity in WT Rats

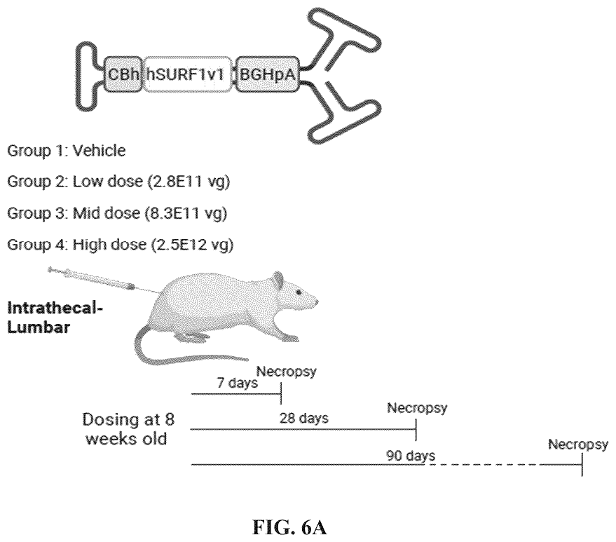

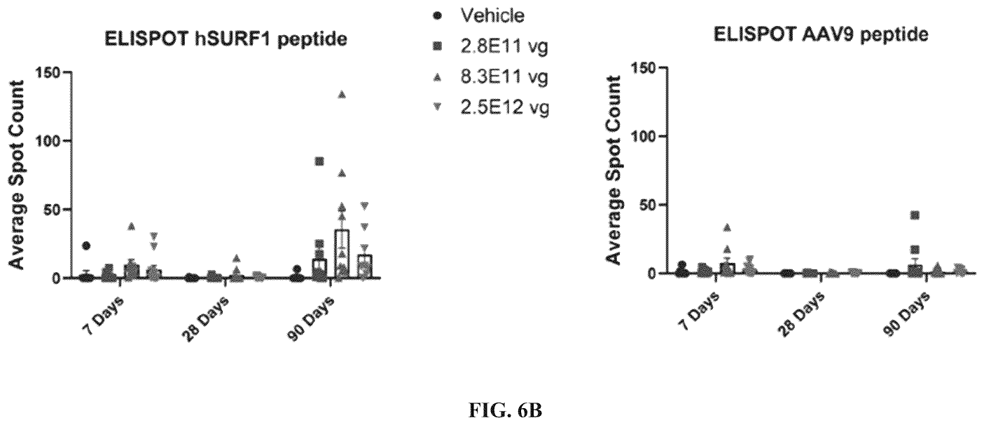

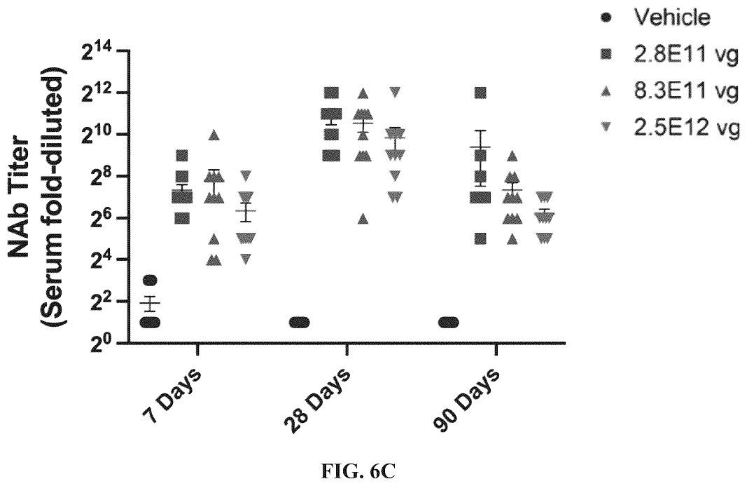

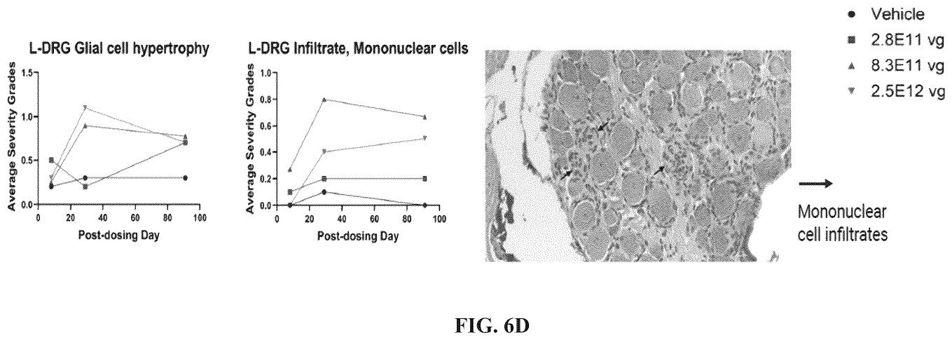

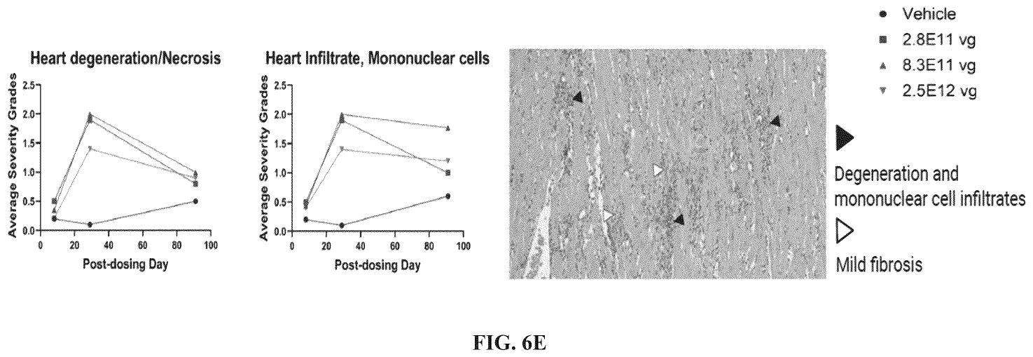

A GLP toxicology study in WT rats, was carried out as a contracted study by Charles River Laboratories. In this study, adult WT rats received either vehicle or AAV9/hSURF1v1, at doses of 2.8E11 vg, 8.3E11 vg or 2.49E12 vg per rat through a single lumbar-intrathecal (IT-LP) injection. Separate groups of animals were dosed and sacrificed for tissue collection at 7 days, 28 days or 90 days post-dosing, as shown in FIG. 6A. It was first examined if there were antigen-related immune responses that may affect transgene expression or induce inflammation. Transduction by AAV9/hSURF1v1 presents two foreign antigens to the immune system of WT rats: the AAV9 capsid and WT human SURF1. To screen for cytotoxic T-cell responses to these antigens, an interferon-gamma (IFN-γ)-based Enzyme-Linked Immunospot (ELISpot) assay for IFN-γ secretion was used after stimulation of splenocytes collected from the treated rats with overlapping peptide pools spanning the AAV9 VP1 capsid protein or WT hSURF1. As shown in FIG. 6B, minimal amounts of IFN-γ secretion were detected in response to the hSURF1 peptide pools at 90 days after treatment while no IFN-γ secretion was detected in response to AAV9 peptide pools. As expected, all vector treatments elicited the production of neutralizing antibodies (NAbs) against AAV9, as shown in FIG. 6C. Histopathological evaluations were carried out in a blinded fashion on various tissues using hematoxylin and eosin (H&E) staining. Signs of cytotoxicities were identified in spinal cord, dorsal root ganglia (DRG), the tibial and sciatic nerves, heart and liver tissues, at all doses tested. Among them, the lumbar DRG and heart showed the most severe changes. FIG. 6D summarizes the average severity of glial cell hypertrophy and mononuclear cell infiltrates in the lumbar DRG at three time points, and a representative H&E staining image. Similarly, in FIG. 6E, the average severity of necrosis and mononuclear cell infiltrates in the cardiomyocytes at three time points is summarized, and a representative image of H&E staining provided. These findings were surprising, since none of these events in the toxicity study in WT mice were identified. To confirm the previous findings in mice, another safety study was conducted by treating WT mice at 4 weeks old administered with 8E11 vg/mouse of AAV9/hSURF1v1 through IT-LP and the tissues examined 28 days post-dosing. Again, no signs of treatment-related cytotoxicity were identified using H&E staining (data not shown). These findings indicate that the treatment AAV9/hSURF1v1 is indeed well-tolerated in mice, but cytotoxicity occurred in rats.

### Example 6: Re-Engineering AAV9/hSURF1 to Reduce Cytotoxicity

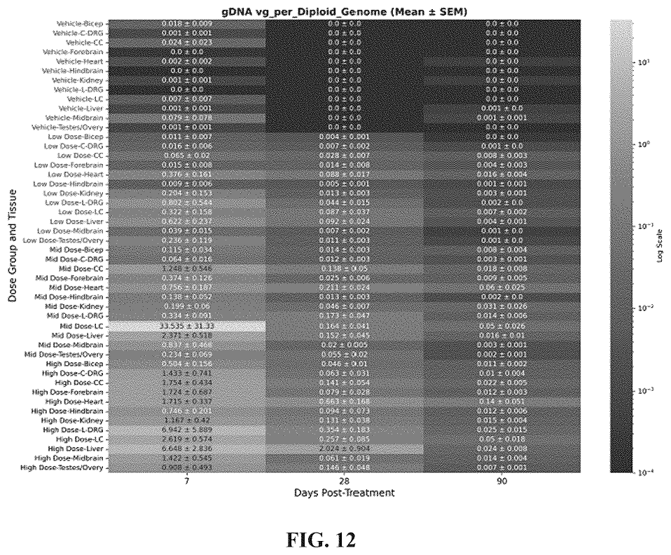

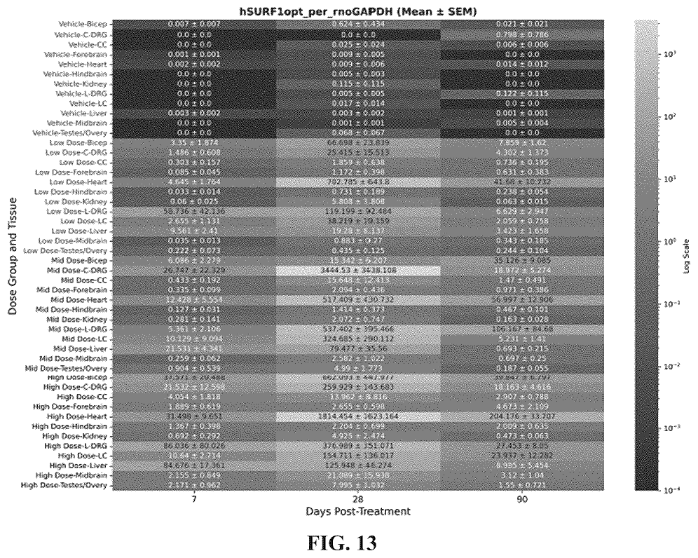

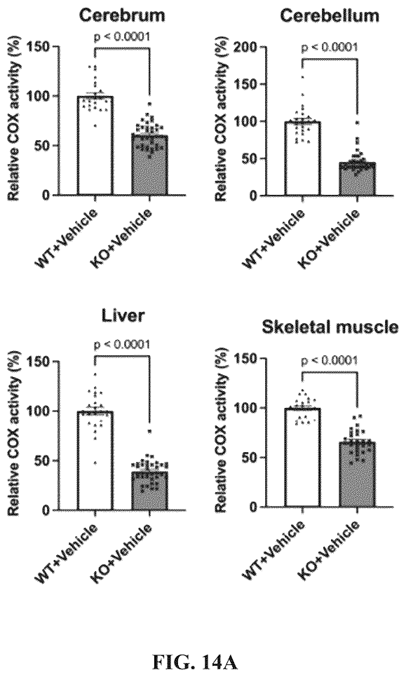

To explore the potential cause of the cytotoxicity shown in the treated rats, biodistribution analysis for both vector genomic DNA (gDNA) and mRNA of hSURF1 on the tissues collected from the rats was conducted (FIG. 12). Compared to 7 days post-dosing, at 30 and 90 days post-dosing the level of gDNA was significantly diminished (FIG. 12). For hSURF1opt mRNA, the highest expression level was observed at 30 days post-dosing, which then declined by 90 days (FIG. 13). Liver and lumbar-DRG showed high gDNA presence and mRNA expression, while the heart (smooth muscle) and bicep (skeletal muscle) showed high mRNA expression despite reduced presence of vector gDNA. This suggests that the high mRNA expression in liver and lumbar DRG was driven by high vector particle transduction, while the high mRNA expression in heart and bicep was from high transcription driven by the CBh promoter. The reduced gDNA presence in all tissues across time, coinciding with the rise in transgene expression, suggests a loss of transduced cells that is likely due to hSURF1opt overexpression. Since the toxicity was seen across all 3 doses, it was concluded that it couldn't be mitigated simply by lowering the vector dose. Rather, it seemed that the CBh promoter-driven expression was producing too much SURF1 protein, particularly in the heart.

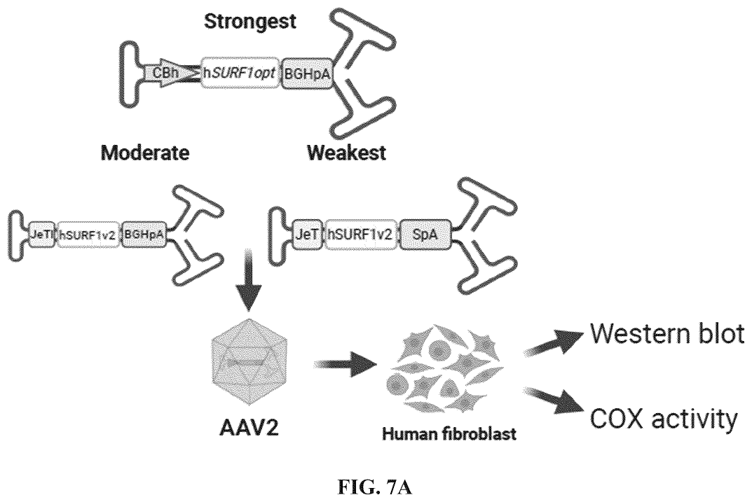

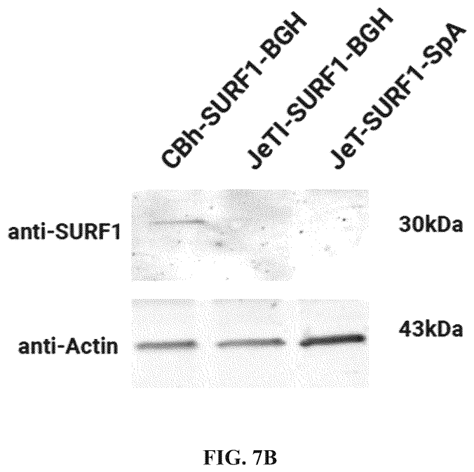

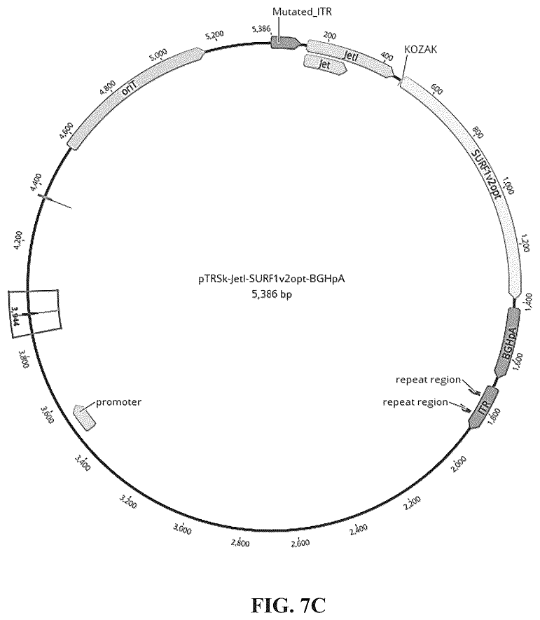

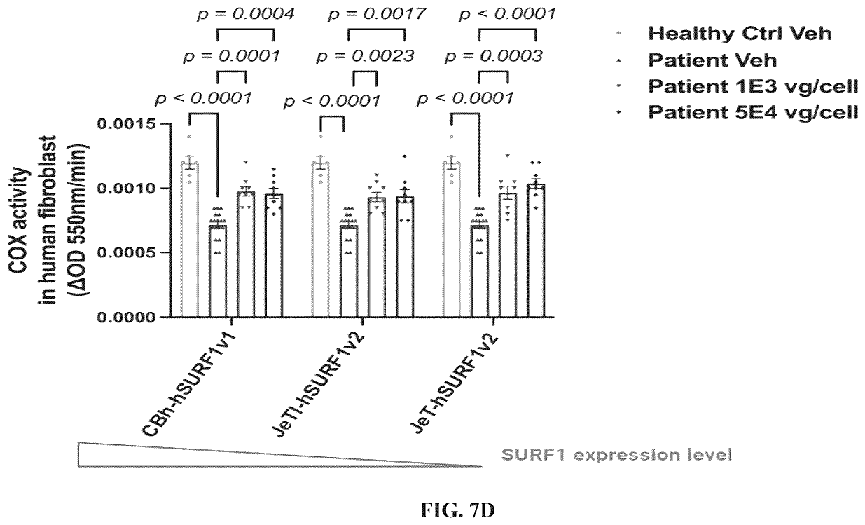

Therefore, to address the toxicity issue, a reasonable step was to reengineer the gene therapy construct to fine tune the expression of the hSURF1 transgene. As shown in FIG. 7A, alternative hSURF1 sequences (hSURF1v2), promoters and polyadenylation signals (polyA) were used to adjust the transgene expression level of two such constructs, one containing stronger elements than the other. The JeT promoter is a synthetic promoter built as a chimeric promoter that merged components of the viral SV40 early promoter, the human beta-actin promoter, and the ubiquitin C promoter. The JeTI, or UsP, promoter was built by adding an intron sequence between the JeT promoter and the transgene start codon. Previous studies have shown that both JeT and JeTI are weaker promoters than CBh, while JeTI is slightly stronger than JeT. Additionally, the weakest construct was designed with the JeT promoter and a synthetic poly (A) (SpA) 16 to drive the lowest expression of hSURF1. These two new constructs are referred to as JeTI-hSURF1v2-M (moderate) and JeT-hSURF1v2-W (weak), respectively. Additionally, the hSURF1 transgene was re-codon-optimized to differentiate it from the previous construct as well as to reduce CpG dinucleotides hSURF1v2.

These constructs were packaged into an AAV2 capsid, and these AAV2/hSURF1 vectors (see FIG. 7C) were tested on patient fibroblasts to evaluate their in vitro efficacy in restoring the COX activity. First, it was examined if the new constructs show lower SURF1 expression compared to CBh-hSURF1v1 using a Western blot. As shown in FIG. 7B, cells transduced with AAV2/CBh-hSURF1v1-BGH showed a clearly visible SURF1 band, while the newer (weaker) constructs did not show any SURF1 protein expression within the sensitivity of this assay. This indicated that the hSURF1v2 designs expressed SURF1 lower than CBh-hSURF1v1, but the assay limitations did not allow for a fold change quantification in the expression. Interestingly, COX activity was restored to a similar level with the original and newer designs, despite the differences in multiplicity of infection (MOI) and transgene expression strength of the construct (FIG. 7D). This result suggested that a lower amount of SURF1 expression were sufficient to normalize COX activity, and there was no added benefit to overexpress SURF1. Therefore, to minimize potential cytotoxicity, hSUR1v2-W was initially selected as a lead candidate for in vivo evaluations.

### Example 7: AAV9/hSURF1v2-W Was Too Weak to Show Biochemical Improvements in Surf1 KO Mice

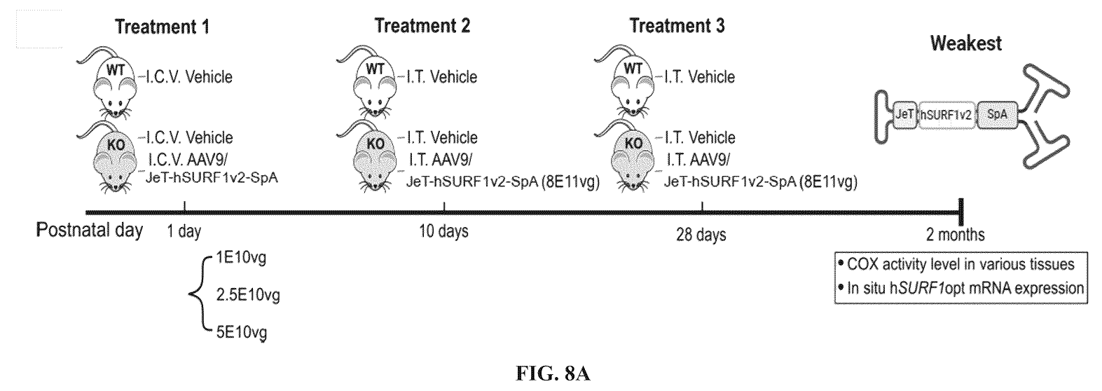

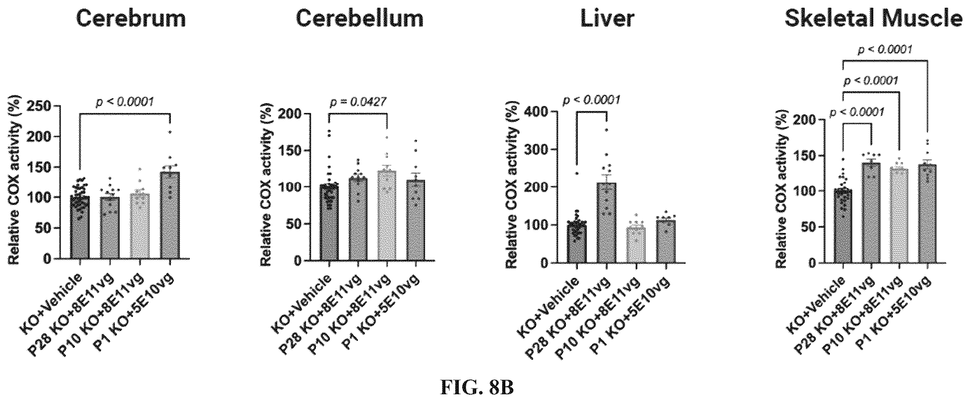

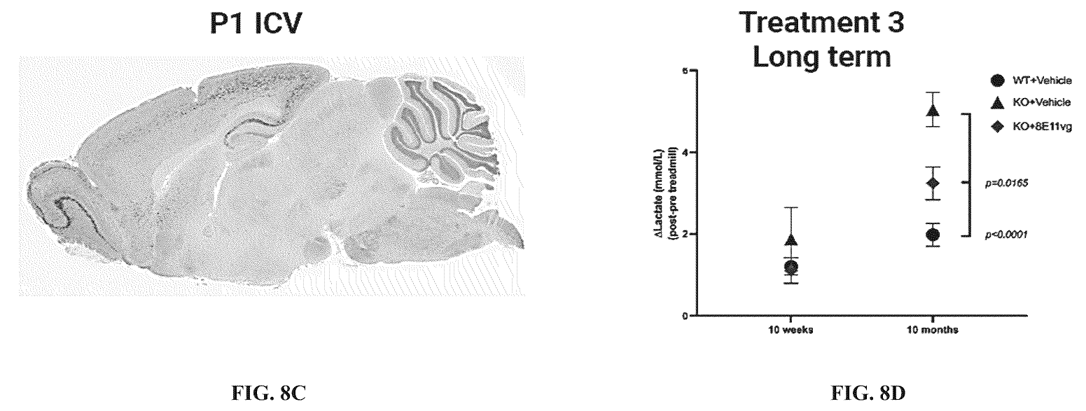

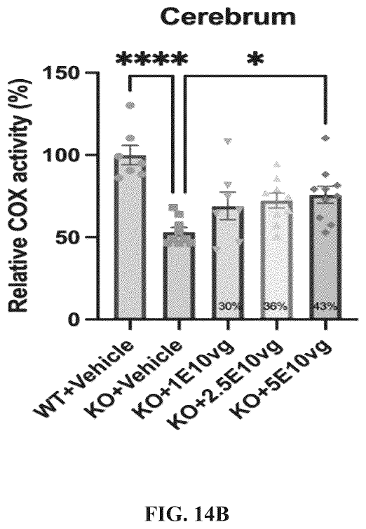

To evaluate the potential of JeT-hSUR1v2 as a gene therapy, the construct was packaged into self-complementary AAV9, and the therapeutic efficacy was evaluated in the Surf1 KO mice. To achieve a more comprehensive and accurate evaluation, different treatment paradigms as described in FIG. 8A were designed. Briefly, IT-LP administration was conducted for both postnatal day PND10 and PND28 mice to compare the efficacy, with the hypothesis that earlier treatment may show improved efficacy due to higher numbers of cells transduced. Additionally, intracerebral ventricular (ICV) injection was conducted with three different doses at PND1, as a proof-of-concept evaluation to examine the degree of the COX activity that can be improved in the brain with the maximum possible number of brain cells transduced. All the animals were sacrificed at two months old for biochemical evaluations. The comparisons between the WT+Vehicle group and the KO+Vehicle group of each tissue are shown in FIG. 14A. As shown in FIG. 8B, the cerebrum showed significantly increased COX activity in the PND1 treated animals (p-value<0.0001 compared with KO+Vehicle group, combined from P28, P10, and P1-treated animals). Only data from the highest dose is shown in FIG. 8, as all three doses showed similar improvements compared with the KO+Vehicle group, but only the 5E10 vg/mouse treatment reached statistical significance on its own (p-value=0.0249) (FIG. 14B). In the cerebellum, only PND10 treated animals showed significantly improved COX activity (p-value=0.0427 compared with KO+Vehicle group), and only PND28 treated animals showed improved COX activity in the liver (p-value<0.0001 compared with KO+Vehicle group). The skeletal muscle exhibited improved COX activity in all treated animals (p-value<0.0001 compared with KO+Vehicle group). FIG. 8C shows a representative brain slice from a PND1 treated mouse stained for hSURF1v2 mRNA using the RNAscope®-based in situ hybridization, which demonstrates that the expression was concentrated in the forebrain regions. This biodistribution pattern explains the differences in COX activity exhibited in cerebrum versus cerebellum from PND1 treated animals, as shown in FIG. 8B.

Additionally, the long-term efficacy for animals treated at PND28 was evaluated by examining the lactate acidosis upon an endurance capacity test. As shown in FIG. 8D, Δlactate (the difference between blood lactate level before and after the endurance capacity test) was significantly increased when the animals were tested at 10 months old, and AAV9/JeT-hSURF1v2 partially reduced the lactate acidosis (p-value<0.0001 compared with the KO+Vehicle group). It is noteworthy that the original CBh-hSURF1v1 vector design completely normalized the lactate acidosis at 10 months old in this mouse model.

### Example 8: IT Delivery of AAV9/hSURF1v2-M Improved COX Activity Deficiency to a Similar Level Exerted by AAV9/hSURF1v1

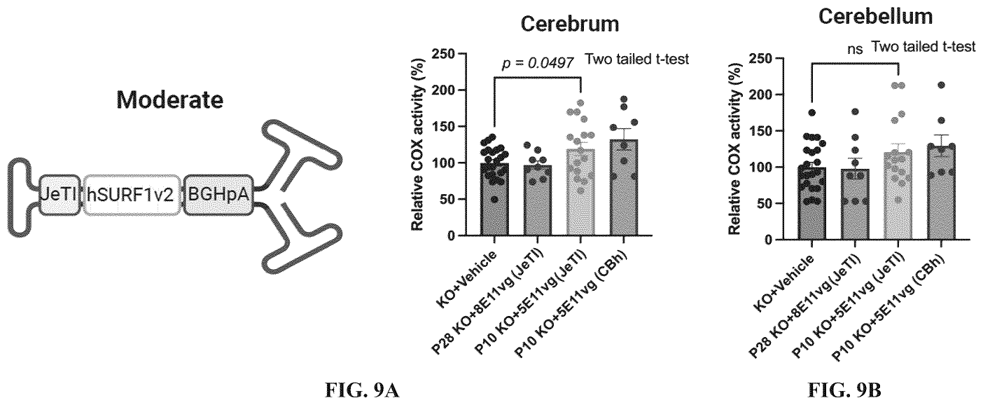

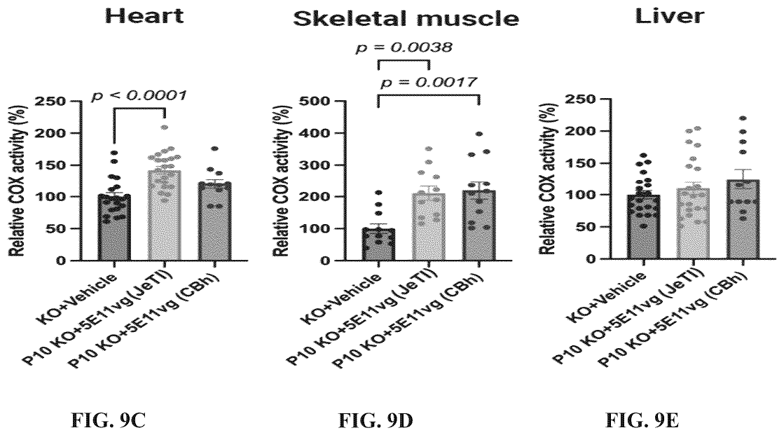

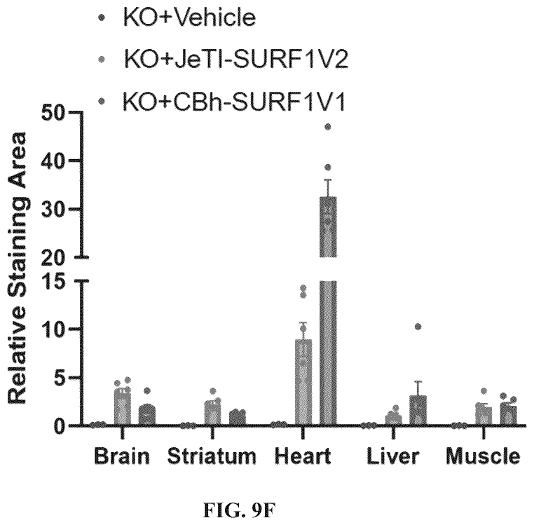

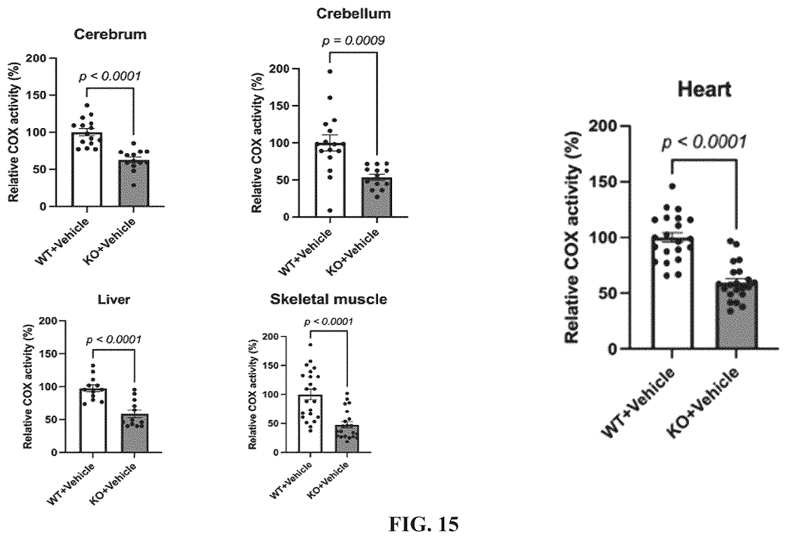

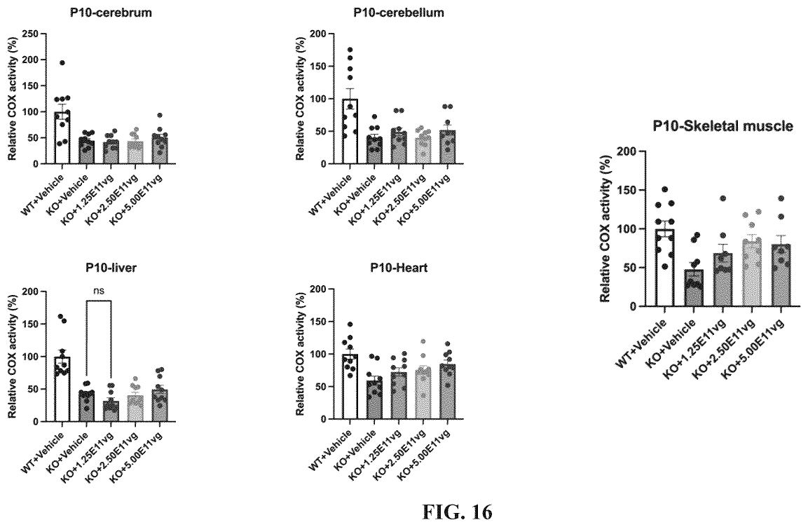

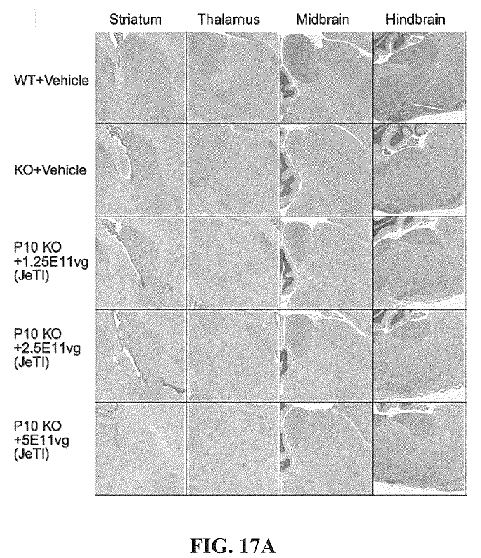

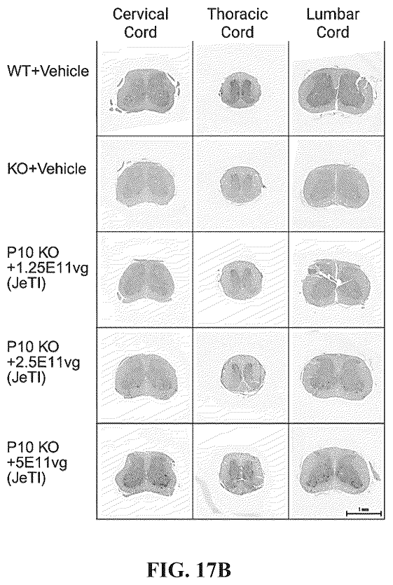

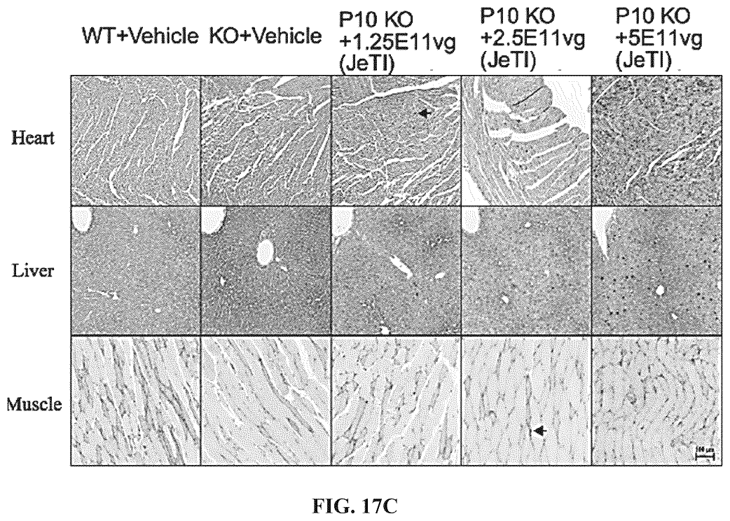

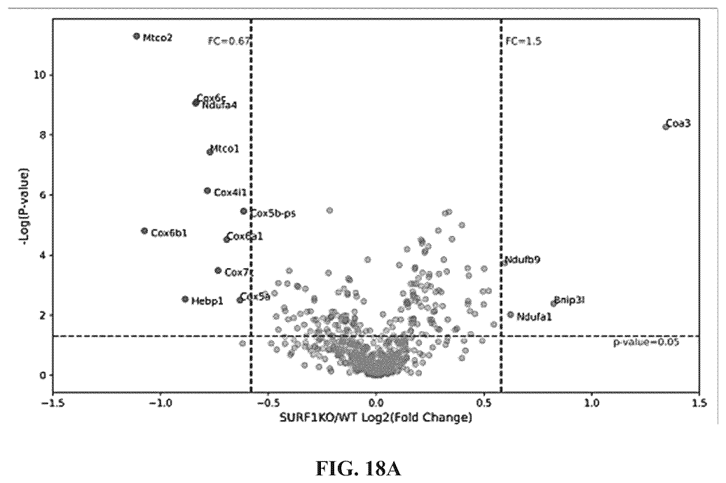

Since Leigh syndrome is a neurodegenerative disease, it is critical that the gene therapy corrects neurological dysfunctions. However, AAV9/JeT-hSURF1v2 did not show improvement in COX activity in the cerebrum with a translatable therapeutic approach, namely the animals that were treated at PND10 or PND28. These data suggested that the protein expression level in each transduced cell was not sufficient to normalize COX activity levels, and there was a need to increase the SURF1 protein expression. Therefore, AAV9/JeTI-hSURF1v2 (AAV9/hSURF1v2-M) was tested through IT-LP administration at PND10 and PND28. To directly compare this design to AAV9/hSURF1v1, AAV9/hSURF1v1 was also administered side-by-side with AAV9/JeTI-hSURF1v2 at PND10. In all experiment groups, the KO+Vehicle group showed between 50% to 40% reduction in COX activity compared to WT mice as shown in FIG. 15. To compare the therapeutic efficacy from both treatment ages and both vectors, the COX activity of all groups was normalized to the average of the KO+Vehicle group. As shown in FIG. 9A, AAV9/JeTI-hSURF1v2 significantly increased cerebrum COX activity compared with the KO+Vehicle group (p-value=0.0497) of the mice treated at PND10, but not PND28. In the cerebellum, AAV9/JeTI-hSURF1v2 increased COX activity by 21% with treatment at PND10 (FIG. 9B). AAV9/JeTI-hSURF1v2 did not show significant differences compared to AAV9/CBh-hSURF1v1 in any of the tissues tested (FIGS. 9A-9E). In heart tissue, AAV9/JeTI-hSURF1v2 showed a trend of higher COX activity than AAV9/CBh-hSURF1v1 treated mice. AAV9/JeTI-hSURF1v2 induced significantly higher COX activity than vehicle-treated animals (p-value<0.0001), while AAV9/hSURF1v1 group did not show statistical significance (FIG. 9C). Additionally, by using RNAscope®, the mRNA expression level of hSURF1 in the mice treated at PND10 was examined. As shown in FIG. 9F and FIG. 17, AAV9/JeTI-hSURF1v2 induced a similar pattern of expression in the brain, liver and muscle, as compared to AAV9/CBh-hSURF1v1. The striatum in the brain was specifically examined (as it is part of the basal ganglia, which is significantly affected in Leigh syndrome), and the expression levels induced by the two vectors were not significantly different. However, AAV9/CBh-hSURF1v1 did show significantly higher expression than AAV9/JeTI-hSURF1v2 in the heart, which was the most problematic tissue for overexpression-related cytotoxicity for AAV9/CBh-hSURF1v1 in rats. Different doses (1.25E11 vg, 2.5E11 vg, and 5E11 vg) of AAV9/JeTI-hSURF1v2 were explored to see if a higher dose is required to show therapeutic efficacy. Data suggested that for the mice treated at PND10, when lower doses are administered (2.5E11vg and 1E11vg), the improvement in COX activity is diminished (FIG. 16). FIGS. 17A-17C shows representative RNAscope® images of target tissue from mice treated with low (1.25E11 vg), mid (2.5E11 vg) or high (5E11 vg) doses of AAV9/SURF1. Spots show increased SURF1opt transgene mRNA expression in a dose dependent manner across all target tissues. Taken together, a high dose and early treatment of AAV9/JeTI-hSURF1v2 through IT-LP effectively improved COX activity in both the central nervous system and in peripheral tissues by SURF1 gene replacement.

### Example 9: Improvements Related to Endurance Capacity and Lactate Acidosis

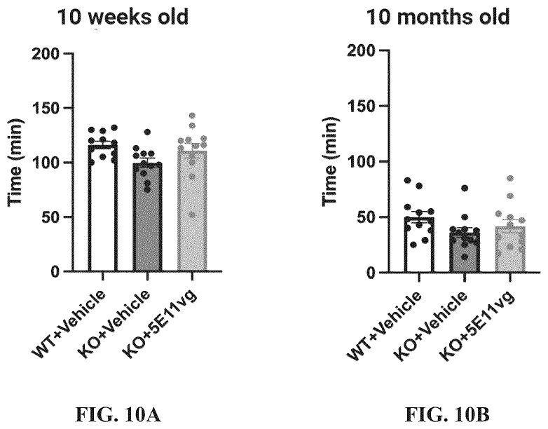

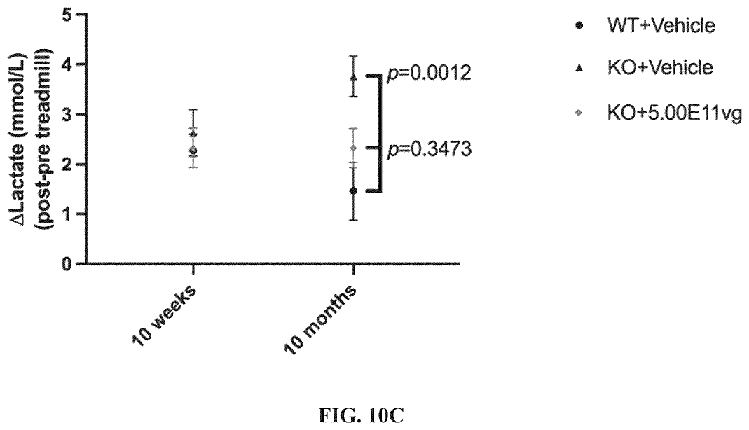

LS patients with SURF1 deficiency often show elevated blood lactate. Previous studies reported that Surf1 KO mice also exhibited elevated blood lactate levels, especially after exhaustive exercise. Thus, to evaluate whether gene therapy can correct the blood lactate level, another group of mice were treated with the same high (5×10¹¹ vg/mouse) dose of AAV9/JeTI-hSURF1v2 through IT administration at PND10 and blood lactate levels examined both at rest and after exhaustive exercise. The mice were tested for endurance capacity at two ages, 10 weeks (9 weeks after treatment) and 10 months (10 months after treatment). Blood lactate was examined both before and after running on a treadmill until exhaustion, and the change in blood lactate level was recorded as ΔLactate. As shown in FIGS. 10A and 10B, the KO+Vehicle group showed a trend of reduced running time to exhaustion at both ages (comparing WT+Vehicle and KO+Vehicle groups, Mean Difference=16.34 min at 10 weeks old, and Mean Difference=13.75 min at 10 months old), and the treatment showed partial improvement (comparing KO+5E11 vg and KO+Vehicle groups, Mean Difference=11.00 min at 10 weeks old, and Mean Difference=5.75 min at 10 months old). Additionally, as shown in FIG. 10C, ΔLactate of Surf1 KO mice was significantly higher than that of WT animals when tested at 10 months of age (p=0.0012). AAV9/JeTI-hSURF1v2 treatment reduced ΔLactate of KO mice closer to the level of WT animals, and the differences between the two groups were not statistically significant (p=0.3473 comparing KO+5E11 vg and WT groups; p=0.0609 comparing KO+5E11 vg and KO+Vehicle group). Taken together, these data suggest that IT-administered AAV9/JeTI-hSURF1v2 restored endurance capacity and mitigated abnormal lactic acidosis during exhaustive exercise in Surf1 KO mice.

### Example 9: IT-LP Delivery of AAV9/hSURF1v2-M is Safe in WT Rats

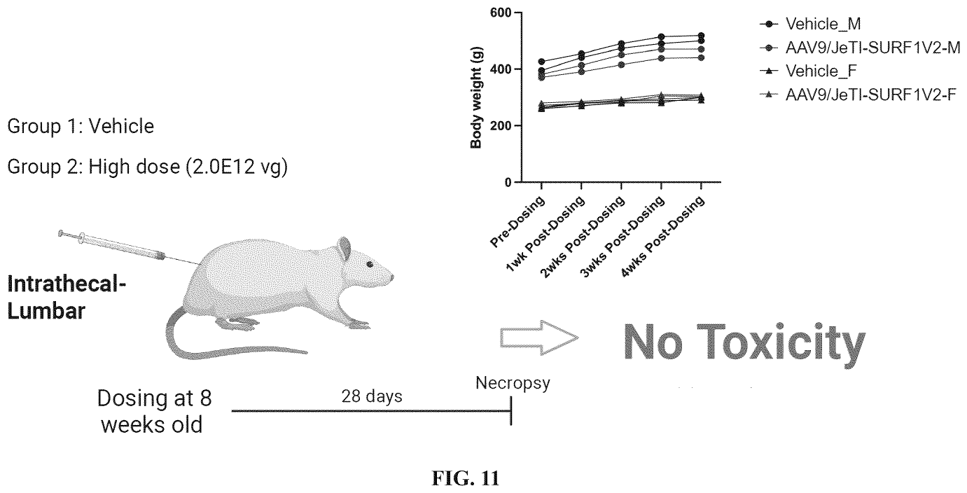

The safety of AAV9/JeTI-hSURF1v2 was evaluated in WT rats by treating 6 weeks old rats with 2E12 vg/rat through IT-LP. First, the overall growth was examined by tracking the body weight changes. As shown in FIG. 11, there was no difference between vehicle and AAV9/JeTI-hSURF1v2 treated rats. Tissue was collected 4 weeks post-dosing, and histopathology evaluations were performed using H&E staining. Treatment with AAV9/JeTI-hSURF1v2 did not lead to any adverse histological findings (Table 1). Thus, it was concluded that this newer design has acceptable safety in rats, and more specifically that the lower expression mediated by the JeTI-hSURF1v2 design overcame the overexpression-related toxicities caused by the original CBh-hSURF1v1 design.

TABLE 1
Non-GLP histopathological evaluation findings

| Rat | | | | | |
|---|---|---|---|---|---|
| ID | Sex | Treatment | Organ | Results | Comments |
| 23 | F | JeTI-SURF1v2 | Brain | Microscopically normal | |
| 24 | F | JeTI-SURF1v2 | Brain | Microscopically normal | |
| 27 | M | JeTI-SURF1v2 | Brain | Microscopically normal | |
| 28 | F | JeTI-SURF1v2 | Brain | Normal | |
| 30 | M | JeTI-SURF1v2 | Brain | Normal | |
| 23 | F | JeTI-SURF1v2 | Sciatic | Few scattered mast | Mast cells are |
| | | | Nerve | cells | frequently present in |
| | | | | | normal, untreated |
| | | | | | animals |
| 24 | F | JeTI-SURF1v2 | Sciatic | Few scattered mast | Mast cells are |
| | | | Nerve | cells | frequently present in |
| | | | | | normal, untreated |
| | | | | | animals |
| 27 | M | JeTI-SURF1v2 | Sciatic | Few scattered mast | Mast cells are |
| | | | Nerve | cells | frequently present in |
| | | | | | normal, untreated |
| | | | | | animals |
| 28 | F | JeTI-SURF1v2 | Sciatic | Normal | |
| | | | Nerve | | |
| 30 | M | JeTI-SURF1v2 | Sciatic | Normal | |
| | | | Nerve | | |
| 23 | F | JeTI-SURF1v2 | Heart | Microscopically normal | |
| 24 | F | JeTI-SURF1v2 | Heart | Microscopically normal | |
| 27 | M | JeTI-SURF1v2 | Heart | Microscopically normal | |
| 28 | F | JeTI-SURF1v2 | Heart | Normal | |
| 30 | M | JeTI-SURF1v2 | Heart | Normal | |
| 23 | F | JeTI-SURF1v2 | Liver | lipidosis-Mild | These findings are |
| | | | | Multifocal; Multifocal | normal and common in |
| | | | | mixed inflammatory | untreated adult rats. |
| | | | | cell infiltrate | |
| | | | | Perivascular and | |
| | | | | Periportal | |
| 24 | F | JeTI-SURF1v2 | Liver | Multifocal mixed | These findings are |
| | | | | inflammatory cell | normal and common in |
| | | | | infiltrates mild | untreated adult rats. |
| | | | | Perivascular and | |
| | | | | Periportal | |
| 27 | M | JeTI-SURF1v2 | Liver | lipidosis-Mild | These findings are |
| | | | | Multifocal; Multifocal | normal and common in |
| | | | | mixed inflammatory | untreated adult rats. |
| | | | | cell infiltrate | |
| | | | | Perivascular and | |
| | | | | Periportal very mild | |
| 28 | F | JeTI-SURF1v2 | Liver | Focal infiltrate with | These findings are |
| | | | | small numbers of | normal and common in |
| | | | | inflammatory cells | untreated adult rats. |
| 30 | M | JeTI-SURF1v2 | Liver | Multifocal interstitial | These findings are |
| | | | | and pervascular | normal and common in |
| | | | | infiltrates with small | untreated adult rats. |
| | | | | numbers of | |
| | | | | inflammatory cells | |
| 28 | F | JeTI-SURF1v2 | Spinal | The sections of cervical | Cause for |
| | | | cord | and thoracic spinal cord | mineralization |
| | | | | contained focal areas of | unknown. |
| | | | | gray matter that contain | |
| | | | | purple/blue foci | |
| | | | | consistent with | |
| | | | | mineralization. The | Cause for |
| | | | | lumbar cord was | mineralization |
| | | | | normal. | |
| 30 | M | JeTI-SURF1v2 | Spinal | A section of thoracic | unknown. |
| | | | cord | spinal cord contained | |
| | | | | multifocal randomly | |
| | | | | scattered areas of gray | |
| | | | | matter that contain | |
| | | | | purple/blue foci | |
| | | | | consistent with | |
| | | | | mineralization. | |
| | | | | Cervical and lumbar | |
| | | | | cord all normal. | |
| 28 | F | JeTI-SURF1v2 | Lumbar | DRG normal. | |
| | | | DRG | | |
| 30 | M | JeTI-SURF1v2 | Lumbar | DRG normal. | |
| | | | DRG | | |
| 21 | F | Vehicle | Brain | Microscopically normal | |
| 25 | M | Vehicle | Brain | Microscopically normal | |
| 29 | F | Vehicle | Brain | Normal | |
| 31 | M | Vehicle | Brain | Normal | |
| 21 | F | Vehicle | Sciatic | NA | |
| | | | Nerve | | |
| 25 | M | Vehicle | Sciatic | Few scattered mast | |
| | | | Nerve | cells | |
| 29 | F | Vehicle | Sciatic | Normal | |
| | | | Nerve | | |
| 31 | M | Vehicle | Sciatic | Normal | |
| | | | Nerve | | |
| 21 | F | Vehicle | Heart | Microscopically normal | |
| 25 | M | Vehicle | Heart | Microscopically normal | |
| 29 | F | Vehicle | Heart | Normal | |
| 31 | M | Vehicle | Heart | Normal | |
| 21 | F | Vehicle | Liver | Multifocal mixed | These findings are |
| | | | | inflammatory cell | normal and common in |
| | | | | infiltrate Perivascular | untreated adult rats. |
| | | | | and Periportal | |
| 25 | M | Vehicle | Liver | Mild Multifocal | These findings are |
| | | | | lipidosis | normal and common in |
| | | | | | untreated adult rats. |
| 29 | F | Vehicle | Liver | Focal infiltrate with | These findings are |
| | | | | small numbers of | normal and common in |
| | | | | inflammatory cells | untreated adult rats. |
| 31 | M | Vehicle | Liver | Focal infiltrate with | These findings are |
| | | | | small numbers of | normal and common in |
| | | | | inflammatory cells | untreated adult rats. |
| 29 | F | Vehicle | Spinal | The section of cervical | Cause for |
| | | | cord | spinal cord contained | mineralization |
| | | | | multifocal randomly | unknown. |
| | | | | scattered areas of gray | |
| | | | | matter that contain | |
| | | | | purple/blue foci | |
| | | | | consistent with | |
| | | | | mineralization. | |
| | | | | Thoracic and lumbar | |
| | | | | cord all normal. DRG | |
| | | | | normal. | |
| 31 | M | Vehicle | Spinal | The sections of cervical | Cause for |
| | | | cord | and thoracic spinal cord | mineralization |
| | | | | contained focal areas of | unknown. |
| | | | | gray matter that contain | |
| | | | | purple/blue foci | |
| | | | | consistent with | |
| | | | | mineralization. The | |
| | | | | lumbar cord was | |
| | | | | normal. DRG normal. | |
| 29 | F | Vehicle | Lumbar | DRG normal. | |
| | | | DRG | | |
| 31 | M | Vehicle | Lumbar | DRG normal. | |
| | | | DRG | | |

### Example 10: Discussion for Examples 5-9

In this study, the previously developed gene therapy for SURF1-Leigh syndrome (as described in Examples 1-4), was re-engineered and it was demonstrated that the new design is equally effective with minimal toxicity. One limitation of developing a gene therapy for SURF1-Leigh syndrome is that the available mouse model does not recapitulate overt phenotypes of the human disease. Thus, the types of outcome readouts for evaluating the efficacy of a gene therapy become limited. The only readouts available so far are COX activity, endurance capacity, and lactate acidosis in the context of the endurance capacity test at 10 months old. For the COX activity assay, bulk tissues were homogenized for mitochondria isolation. However, since not all cells are transduced by AAV9 to an equal extent, and SURF1 is a non-secretive protein, the results of the assay encompass cells of different SURF1 transgene expression levels, resulting in different degrees of restoration in COX activity. Consequently, the COX activity values presented are an average of all cells (including non-transduced cells), some of which show better restoration than others. Based on the biodistribution data of self-complementary AAV9 and the data acquired from WT rats, the peripheral organs that are affected in the disease, including heart, liver and skeletal muscle, have been well-transduced. The normalization of endurance capacity and lactic acidosis at 10 months old further supported the gene therapy efficacy in the skeletal muscle. However, the current animal model makes it difficult to determine whether the number of cells targeted with the current therapy is enough to provide any neurological benefits. A recent publication provided promising indications that transfecting less than 30% cells lead to robust phenotypic restoration in two mitochondrial encephalopathy models, suggesting that the increases in COX activity achieved in this study is sufficient to predict a meaningful therapeutic benefit.

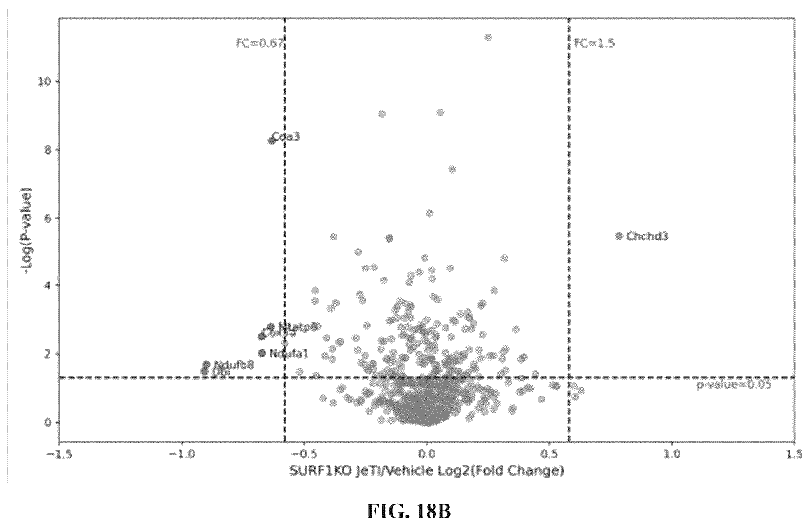

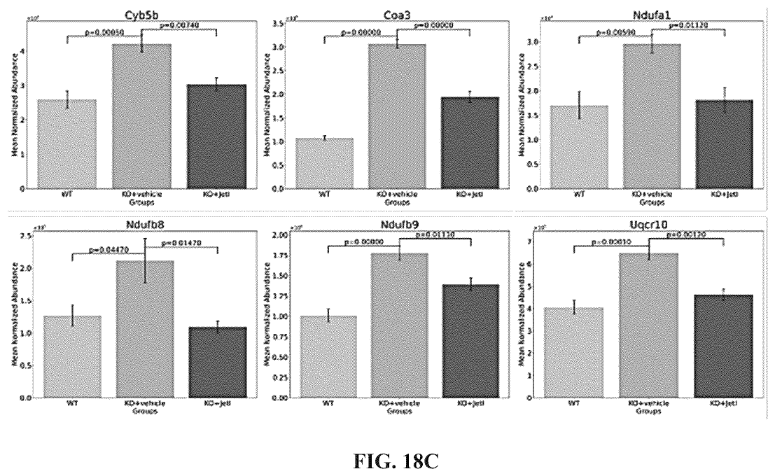

The protein expression profile of the cerebrum of PND10-treated animals where tissues were collected at PND56 were also analyzed. The proteomic data provided further complementary evidence on the restoration of the normal cell state with the gene therapy treatment. COA3 has been suggested as an assembly factor for the COX1 subunit of the COX holoenzyme. NDUFB9, NDUFA1 and NDUFB8 are subunits of complex I, while UQCR10 is a subunit of complex III. CYB5B is a protein that enables heme binding activity. The expression of each was significantly increased in Surf1 KO mice, possibly due to a negative feedback mechanism, and these increases were normalized to the level of WT mice with AAV9/JeTI-hSURF1v2 treatment (FIG. 18A-18C). However, Mtco1 and Mtco2 encoded proteins, two core subunits of COX, showed reduced expression in Surf1 KO mice, but the gene therapy treatment was only able to exert a slight increase of these proteins (6.0% for Mtco1 and 17.1% for Mtco2, not statistically significant). These data complement and reinforce the COX activity data, indicating that the gene therapy treatment was able to partially restore protein expression homeostasis in the cerebrum of Surf1 KO mice.

This study presents an example where the efficacy of gene therapy is dictated by both vector transduction and the transgene expression regulated by the transgene cassette. They work hand-in-hand exhibiting a “bell curved” relationship, where too little transduction or gene expression leads to suboptimal therapeutic efficacy, while too much transduction and transgene overexpression may lead to cytotoxicity. Mitochondria transfer between neighboring cells occurs as a cellular repair mechanism, instead of a physiological process of constant mitochondria exchange between cells. Therefore, gene therapy for mitochondrial diseases may be expected to be cellular autonomous, meaning only cells that are transduced with the vector are treated.

However, for tissues where vector transduction efficiency is high, the gene expression level can be modulated by the transgene cassette design, and a high expression vector can lead to cytotoxicity. This conclusion was supported by the GLP toxicology study with the original AAV9/CBh-hSURF1v1, in which high hSURF1 mRNA expression led to cell degeneration and mononuclear cell infiltrates in multiple tissues, especially the heart and lumbar-DRG. AAV9 delivered through IT-LP has been shown to cause similar DRG-related cytotoxicities in several other studies. However, the cytotoxicities in the heart were particularly concerning, warranting further investigation. In the disclosed studies of Surf1 KO mice, it was observed that COX activity of the heart trended lower in AAV9/CBh-hSURF1-v1 treated animals compared with those treated with AAV9/JeTI-hSURF1v2. Accordingly, as shown in FIG. 9F, mRNA of hSURF1 was about 3-fold higher in the heart of AAV9/CBh-hSURF1v1 treated animals than AAV9/JeTI-hSURF1v2treated animals. Both studies suggested that the CBh promoter preferentially drives strong expression in cardiomyocytes, and this transgene overexpression presumably creates mitochondrial stress, leading to cytotoxicity. However, this is not the first mitochondrial disease that raised this issue. Studies in gene therapy for Friedreich's ataxia also showed toxicity in the mouse model.

### Example 11: Materials and Methods

### Animals

The Surf1 KO mice were backcrossed with WT C57BL/6J mice for 10 generations. Thus, the Surf1 KO mice in this study are on a C57BL/6J genetic background. The genotypes were determined by PCR analysis using a mouse ear punch. The WT C57BL/6J mice were purchased from the Jackson laboratory (Bar Harbor, ME). The WT Sprague-Dawley rats were purchased from Charles River Laboratory. The animal studies at UTSW were conducted according to protocols approved by the Institutional Animal Care and Use Committee (IACUC). All mice and rats were weaned between PND21-28. All mice and rats were provided food and water ad libitum.

### Plasmids

The complete name for hSURF1v1 is self-complementary CBh-codon-optimized human SURF1-BGHpA. It is composed of a CBh promoter, a codon-optimized human SURF1 cDNA sequence (ATUM, Newark, CA) and a BGH poly (A) tail. The 5′-ITR is mutated to form the self-complementary structure.

For JeT-hSURF1v2 and JeTI-hSURF1v2, the CBh promoter was replaced with the JeT or JeTI promoter (respectively). The JeTI promoter is the JeT promoter with an added intron and is also referred to as the UsP promoter. Additionally, the human SURF1 cDNA sequence was codon-optimized again (ATUM, Newark, CA) to reduce CpG dinucleotides. For JeT-hSURF1v2, the poly (A) was replaced with a synthetic poly(A) (SpA) sequence.

### Virus Production

AAV9/JeTI-hSURF1v2 and all AAV2 vectors were produced at the University of North Carolina-Chapel Hill Vector Core as described.38 Purified vectors were dialyzed in PBS (350 mM final NaCl concentration) containing 5% D-sorbitol and stored at −80° C. until use. Thawed aliquots were subsequently stored at 4° C. A filter-sterilized solution of PBS (350 mM final NaCl concentration) containing 5% D-sorbitol was used as vehicle and virus dilution buffer. The viral vector was titered by qPCR and confirmed by PAGE and silver stain by the UNC Vector Core.39 The vectors in this study were packaged in self-complementary AAV9. This preparation was re-titered by the University of Texas Southwestern Translational Gene Therapy Core (UTSW TGTC), to ensure that comparable doses were used across all studies.

AAV9/JeT-hSURF1v2 and AAV9/CBh-hSURF1v1 were produced by the UTSW TGTC and formulated in phosphate-buffered saline containing 5% d-sorbitol and 0.001% pluronic F-68. It was produced by triple transfection of suspension-cultured HEK293 cells, followed by cell lysis and recovery of recombinant AAV virus from the cells and media. Purification was through filtration, affinity chromatography, and anion exchange chromatography using methods developed at the TGTC. The vector was titered by quantitative PCR directed to the ITR.

### Mouse and Rat IT-LP Injections

AAV9 vectors were diluted in vehicle solution. The mouse IT-LP injections were conducted through lumbar puncture as previously described, with volumes ranging from 7.3 μL to 16.8 μL.40 For ICV injection, the mouse pup was cryo-anesthetized using a barrier to prevent direct contact with the cold source and to keep the pup dry. Lack of response to toe pinch was utilized to ensure the pup reached the correct anesthetic depth. The pup was placed on chilled modeling clay to hold the pup in place and prolong anesthesia during injection. A 33 gauge needle with needle stop attached to a Hamilton syringe was inserted to a depth of 2 mm and used to deliver 1.25 μL of AAV per hemisphere at a rate of 1-2 μL/min into the lateral ventricle. The needle was removed 15 seconds after discontinuation of plunger movement to prevent backflow. Mice were then placed on top of a warming pad and returned to the home cage with the dam after regaining full activity typical of newborn mice. For IT-LP injections in rats, each rat was induced and maintained under isoflurane gas anesthesia for the duration of the injection procedure. The dorsal lumbar region was shaved, and the skin was prepped using 70% isopropyl alcohol, 2% chlorhexidine solution and povidone-iodine. A 1 cm incision in the skin was made over the L5-6 junction. A 1-inch, 27-gauge needle attached to a 100 μL Hamilton syringe was inserted between the vertebrae and into the intrathecal space. The vehicle or the vector solution was delivered at a slow, continuous rate in a volume of 44 μL. The needle was removed 30 seconds after discontinuation of the plunger movement to prevent backflow. The skin incision was closed with suture. Rats were recovered from anesthesia in a warmed recovery chamber until normal ambulation was regained, then returned to the home cage. Rats were monitored and provided pain medication post operatively in accordance with institutional and IACUC regulations.

### Safety Study in WT CD Rats in a GLP Study

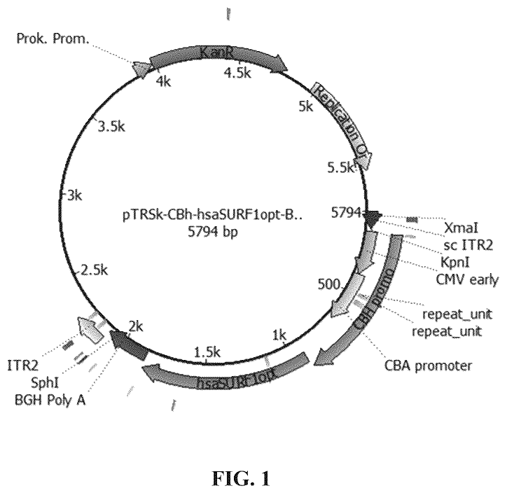

This animal study was performed by Charles River Laboratories according to Good Laboratory Practice (GLP), with immunology and biodistribution studies carried by Dr. Gray's laboratory as GLP exceptions. Male and female SD rats were randomized into cohorts, with 5 males and 5 females per cohort, and dosed as shown in FIG. 1A. At the initiation of dosing, the animals assigned to the study were approximately 56 to 63 days old and weighed between 165 g and 328 g. The AAV9/hSURF1v1 vector was injected IT-LP once in each animal by a qualified laboratory technician, in a volume of 20 or 60 μL at a final dose of 2.8E11, 8.3E11, or 2.5E12 vg/rat. All animals were monitored up to 90 days following the injection. Rats were sacrificed on day 7, 28, or 90 after injection, and tissues were collected for biodistribution and histopathology evaluation. For biodistribution, both mRNA and total genomic DNA were purified from tissue samples collected at necropsy day 28, using a QIAGEN QIAcube HT. mRNA was then reverse-transcribed into cDNA using QIAGEN RT2 HT first strand kit. qPCR was used to determine the quantity of the hSURF1 transgene per diploid rat genome (DNA analysis) or copies of hSURF1 per copy rat GAPDH (mRNA analysis). Details of this study are provided in Charles River Laboratories' final report, provided as supplemental material.

### ELISpot Analysis

Splenocytes were isolated at Charles River Laboratory from treated WT rats, and stored in freezing medium (90% FBS, 10% DMSO), in the vapor phase of liquid nitrogen until the ELISpot assay.

For preparation of the peptide pools, peptides were ordered from Mimotopes (Victoria, Australia). The AAV9 capsid pools were both AAV9 capsid comprised of 20-mers with a 10 amino acid offset, and hSURF1 peptide pools were comprised of 15-mers with a 5 amino acid offset. The AAV9 capsid library pool contained 146 peptides and the final concentration of each peptide in the pool was 0.63 mg/mL in 1.59% DMSO final. The hSURF1 peptide library pool contained 29 peptides and the final concentration of each peptide in the pool was 1.02 mg/mL in 0.61% DMSO final. Both pools were stored at −80° C.

ELISpot assays were performed using an ImmunoSpot kit (mIFNg-1M/5, Cellular Technology Limited). Briefly, splenocytes were thawed, washed, and resuspended in cRMPI-1640 medium for counting. 2E5 splenocytes in 100 μL of cRPMI-1640 medium were plated into each well of an ELISpot plate in quintuplicates whenever possible. 100 μL of cRPMI-1640 medium containing AAV9 capsid or hSURF1 peptide pool was then added to the wells. The controls included cells with no peptide, cells stimulated with a mixture of Phorbol 12-myristate 13-acetate (PMA) and Ionomycin, (Invitrogen, 00-4970-93), medium with INFγ which was supplied with the kit, or medium only. The splenocytes were incubated for 48 h in a humidified 37°° C. CO2 incubator. All other steps were performed according to the manufacturer's recommendation. Spots were read with the same sensitivity settings for all plates.

### Neutralizing Antibody Assay

AAV9/CBh-EGFP as a reporter vector was incubated with serial dilutions of serum samples from each rat and then added to Lec2 cells seeded at 6.5E3 cells per well of a 96-well plate. ADK9 antibody (Progen, Wayne, PA) was incubated with serial dilution as a positive control for inhibition. AAV9 was added at an MOI of 3.9E9 vg/well. The infection efficiency was estimated 48 h later via EGFP intensity in infected cells measured using a microplate reader. AAV NAb titers were determined as the dilution of the tested serum that resulting in ˜50% GFP expression relative to the uninhibited control. Cells not infected with AAV were used to establish the baseline (background) fluorescent signal.

### Cell Culture and AAV Transduction

Human fibroblasts were cultured in complete culture media consisting of 50% Dulbecco's Modified Eagle Medium (+4.5% D-glucose, +L-glutamine,-Sodium Pyruvate), 50% Minimum Essential Media 1-a (1×), 1% Penicillin/Streptomycin (10,000 U/mL), 15% fetal bovine serum (FBS), and 1% GlutaMax (1×). The cells were maintained in a humidified 5% CO2 incubator at 37° C. 24h before AAV transduction, cells were seeded into a T150 flask, and AAV vectors were added into the culture 24h later based on the MOI described in the Results. Cells were collected 48 h after transduction using 0.05% Trypsin-EDTA, and cell pellets were stored at −80° C. for COX activity assay.

### COX Activity Assay for Human Fibroblast

The COX Assay was performed using the Complex IV/COX Human Enzyme activity microplate assay kit (Abcam, Cambridge, MA), following the manufacturer's instructions. Briefly, depending on the estimated pellet size, each sample pellet was resuspended in 50 to 100 mL of Solution 1. Protein concentration of each sample was determined using a BCA assay, and the final protein concentration was adjusted to 5 mg/mL for each sample. A mild detergent was added to each sample to a final dilution of 1:10, and each sample was incubated on ice for 30 minutes, to break the mitochondrial membrane. The samples were then centrifuged for 20 minutes at 12,000×g at 4° C. The supernatant was collected and diluted with Solution 1, and COX was immunocaptured in a pre-coated 96-well plate. COX activity is determined colorimetrically through the oxidation of reduced cytochrome c as an absorbance decrease at 550 nm. The absorbance of each sample was measured once every minute for 120 minutes and the initial rate of decrease was calculated as COX activity.

### Western Blot

SURF1 patient fibroblast was seeded at 3.8E4 cells/well in 6 well plate the day before AAV2 transduction. 24 h later, AAV2/hSURF1 were added into the culture wells at 5E4 vg/cell. After transduction for 48 h, cells were collected for Western blot. Protein was extracted using RIPA buffer (Thermo Scientific, Waltham, MA), and total protein concentration was determined using BCA assay. Equal amounts of protein were separated by gel electrophoresis using 4-20% mini-PROTEAN TGX precast protein gels (Bio-Rad, Hercules, CA). Proteins were transferred to a PVDF membrane, and upon overnight blocking with 5% blocking buffer (Bio-Rad, Hercules, CA), SURF1 protein was detected using SURF1 primary antibody (GENETEX, Irvine, CA) at 1:500 dilution and goat anti-rabbit Dylight 680 secondary antibody (Thermo Scientific, Waltham, MA) at 1:1000 dilution. Membrane was then incubated with actin-Dylight 680 loading control (Thermo Scientific, Waltham, MA) at 1:1000 overnight at 40C as internal reference.

### COX Activity Assay for Mouse Tissues

Mice were perfused with 1×PBS 4 weeks post-treatment. Tissues were extracted quickly and immediately frozen on dry ice. All tissues were stored at −80° C. until use. Complex IV/COX Rodent Enzyme activity microplate assay kit (Abcam, Cambridge, MA) were used to examine the COX activity of the mice model, according to the manufacturer's instructions. Mitochondria were extracted. After mitochondria extraction, COX is immunocaptured within the wells and activity is determined colorimetrically through the oxidation of reduced cytochrome c as an absorbance decrease at 550 nm. The absorbance of each sample was measured once every minute for 120 minutes and the initial rate of decrease was calculated as COX activity. For COX activity of 8-week old mice, assays were performed in multiple batches, in which at least one WT and one KO+Vehicle mouse were included. Each data point was then normalized to the average of all WT COX activity samples measured in that set, as relative COX activity. For COX activity of 18-month old mice, assays were performed in three batches for each tissue. It was noticed that there were significant differences among the raw values of WT COX activity of the three batches. Thus, each data point was normalized to the average of WT COX activity within each batch as relative COX activity.

### Exhaustive Exercise on Treadmill and Blood Lactate Test

Blood lactate following exhaustive exercise was measured by the UTSW Metabolic Phenotyping Core. All mice were familiarized to the treadmills for 2 days prior to the exercise session. On day 1, they experienced a 5 min rest on the treadmill followed by running for 5 min at the speed of 8 m/min and then for 5 min at the speed of 10 m/min. On day 2, they experienced a 5 min rest on the treadmill followed by running for 5 min at the speed of 10 m/min and then for 5 min at the speed of 12 m/min.

On Day 3, mice were placed on the treadmill for 5 min at rest, followed by running at a starting speed of 10 m/min for 40 min, next by running at speeds that were increased at the rate of 1 m/min every 10 min until the speed reached 13 m/min, and finally by running at speeds that were increased at the rate of 1 m/min every 5 min until exhaustion. The exhaustion time was noted as the time at which the mice remained on the electric shock grid for 5 continuous seconds, without attempting to resume running. Blood lactate was taken before putting the mice onto the treadmill and immediately when the mice came off the treadmill. A drop of blood was collected from the tail vein and blood lactate concentration was measured using a Lactate Plus lactate meter (Nova Biomedical, Waltham, MA).

### Proteomics

Proteins from isolated mitochondria were extracted with RIPA buffer, and the protein concentration was determined using BCA assay. 2.5 μg/μl protein sample of 50 μg total protein was used for the analysis. Following disulfide bond reduction and alkylation, samples were digested overnight with trypsin using an S-Trap (Protifi, Fairport, NY). The peptide eluate from the S-Trap was dried and reconstituted in 100 mM TEAB buffer. The TMTpro18plex Isobaric Mass Tagging Kit (Thermo Scientific, Waltham, MA) was used to label the samples as per the manufacturer's instructions. The combined sample then underwent solid-phase extraction cleanup with an Oasis HLB plate (Waters, Milford, MA) and was dried in a SpeedVac. The sample was then reconstituted in a 2% acetonitrile, 0.1% TFA buffer and diluted such that about 1 μg of peptides was injected. Peptides were analyzed on a Thermo Orbitrap Eclipse MS system coupled to an Ultimate 3000 RSLC-Nano liquid chromatography system. Samples were injected onto a 75 um i.d., 75-cm long EasySpray column (Thermo Scientific, Waltham, MA) and eluted with a gradient from 0-28% buffer B over 180 min at a flow rate of 250 nL/min. Buffer A contained 2% (v/v) ACN and 0.1% formic acid in water, and buffer B contained 80% (v/v) ACN, 10% (v/v) trifluoroethanol, and 0.1% formic acid in water at a flow rate of 250 nl/min. Spectra were continuously acquired in a data-dependent manner throughout the gradient, acquiring a full scan in the Orbitrap (at 120,000 resolution with a standard AGC target) followed by MS/MS scans on the most abundant ions in 2.5 s in the ion trap (turbo scan type with an intensity threshold of 5,000, CID collision energy of 35%, standard AGC target, maximum injection time of 35 ms and isolation width of 0.7 m/z). Charge states from 2-6 were included. Dynamic exclusion was enabled with a repeat count of 1, an exclusion duration of 25 s and an exclusion mass width of ±10 ppm. Real-time search was used for selection of peaks for SPS-MS3 analysis, performed against a list of mouse mitochondrial proteins from UniProt along with additional mouse mitochondrial proteins. Up to 2 missed tryptic cleavages were allowed, with carbamidomethylation (+57.0215) of cysteine and TMTpro reagent (+304.2071) of lysine and peptide N-termini used as static modifications and oxidation (+15.9949) of methionine used as a variable modification. MS3 data were collected for up to 10, MS2 peaks which matched to fragments from the real-time peptide search identification, in the Orbitrap at a resolution of 50,000, HCD collision energy of 65% and a scan range of 100-500.

Protein identification and quantification were done using Proteome Discoverer v.3.0 SP1 (Thermo Scientific, Waltham, MA). Raw MS data files were analyzed against the human reviewed protein database from UniProt. Both Comet and SequestHT with INFERYS Rescoring were used, with carbamidomethylation (+57.0215) of cysteine and TMTpro reagent (+304.2071) of lysine and peptide N-termini used as static modifications and oxidation (+15.9949) of methionine used as a variable modification. Reporter ion intensities were reported, with further normalization performed by using the total intensity in each channel to correct discrepancies in sample amount in each channel. The false-discovery rate (FDR) cutoff was 1% for all peptides.

### SURF1 RNAscope®

SURF1 RNA was examined in situ for brain, heart, liver, and skeletal muscle from a set of treated animals. The following protocol was carried out by a senior histologist in the Gray lab, blinded to the treatments and genotypes of all animals. Tissues ready for staining were processed and embedded in paraffin and cut into 5 μm sections on slides. RNAscope® 2.5 HD Assay kit (Advanced Cell Diagnostics, Newark, CA) was used, with a custom probe designed against the hSURF1v1 or hSURF1v2 sequences. Slides were deparaffinated by xylene and then xylene was removed with 200 proof ethanol. Then slides were incubated with hydrogen peroxide for 10 minutes at room temperature and washed with distilled water. Antigen retrieval was performed by boiling the slides in 1× Target Retrieval solution for 10 minutes, washing with distilled water, dehydrated with 200 proof ethanol, and then air dried. Protease Plus was added to each section, incubated at 40° C. for 30 minutes, and washed with distilled water. Then slides were incubated with hSUR1opt RNAscope® probe in HybEZ Oven at 40° C. for 2 hours and washed with 1× Wash Buffer. Then slides were incubated with AMP 1-6 for 30 or 15 minutes following the RNAscope® 2.5 HD Detection Kit protocol. Then, slides were incubated with RED solution for 10 minutes to detect the RNAscope® signals. Finally, slides were covered with coverslips and imaged with Aperio imagescope.

### Image Analysis

Histology images were analyzed using custom analysis settings in HALO™ Image Analysis Platform (Halo2.2, Indica Labs, Albuquerque, NM). A region of interest (ROI) was hand drawn on each image to allow for analysis by tissue region; For the brain, the entire brain was analyzed, including cerebrum and cerebellum, and performed a separate analysis for striatum. A threshold for each stain was set using positive and negative control images, and the same analysis settings were applied for every image of the same stain. Percent area strongly staining for each marker of interest was recorded for each tissue/ROI.

### Statistical Analysis

Underlying assumptions checking for the continuous variables was performed prior to analysis. Specifically, Shapiro-Wilk test for the normality of the data distribution and Brown-Forsythe test for homogeneity of variance were used. Univariable comparisons were conducted using Student's t-test or one-way ANOVA for normally distributed data with equal variance, and Wilcoxon or Kruskal-Wallis test, as a non-parametric counterpart, for non-normal data. A statistical significance was assumed at the 0.05 level, and multiple comparisons were adjusted using Tukey correction or Dunn's multiple comparisons test. Blood lactate data were compared with two-way ANOVA with adjustment of multiple comparisons using Tukey's multiple comparisons test. Graphpad Prism (GraphPad Software, Inc., San Diego, CA) was used for all statistical analysis and generating the graphs.

What is claimed is:

1. A transgene nucleic acid molecule, comprising a nucleic acid sequence as set forth in SEQ ID NO: 22, or a sequence at least 90% identical thereto, operably linked to a promoter sequence.

2. The transgene nucleic acid molecule of claim 1, wherein the transgene nucleic acid molecule comprises a nucleic acid sequence as set forth in SEQ ID NO: 22.

3. The transgene nucleic acid molecule of claim 1, wherein the transgenic nucleic acid molecule encodes a SURF1 polypeptide.

4. The transgene nucleic acid molecule of claim 1, wherein the promoter comprises a JeT promoter, a UsP promoter (JeTI), a Rous sarcoma virus (RSV) LTR promoter, a cytomegalovirus (CMV) promoter, an SV40 promoter, a dihydrofolate reductase promoter, a beta-actin promoter, a phosphoglycerol kinase (PGK) promoter, a U6 promoter, an H1 promoter, a CAG promoter, a hybrid chicken beta-actin promoter, an MeCP2 promoter, an EF1 promoter, a ubiquitous chicken β-actin hybrid (CBh) promoter, a U1a promoter, a Ulb promoter, an MeCP2 promoter, an MeP418 promoter, an MeP426 promoter, a minimal MeCP2 promoter, a VMD2 promoter, an mRho promoter, EF1a promoter, Ubc promoter, human β-actin promoter, TRE promoter, Ac5 promoter, Polyhedrin promoter, CaMKIIa promoter, Gal1 promoter, TEF1 promoter, GDS promoter, ADH1 promoter, Ubi promoter, or α-1-antitrypsin (hAAT) promoter.

5. The transgene nucleic acid of claim 4, wherein the promoter sequence comprises a nucleic acid sequence set forth in SEQ ID NO: 14 (JeT), SEQ ID NO: 20 (cBh), or SEQ ID NO: 23 (UsP/JeT1).

6. The transgene nucleic acid of claim 5, wherein the promoter sequence comprises a nucleic acid sequence set forth in SEQ ID NO: 23.

7. The transgene nucleic acid of claim 1, further comprising a polyA sequence operably linked to the nucleic acid sequence.

8. The transgene nucleic acid of claim 7, wherein the polyA sequence comprises a nucleic acid sequence set forth in SEQ ID NO: 21 or SEQ ID NO: 24.

9. The transgene nucleic acid molecule of claim 1, comprising, in the 5′ to 3′ direction
a) the promoter sequence, wherein the promoter sequences comprises the nucleic acid sequence set forth in SEQ ID NO: 14 or SEQ ID NO: 23, or a sequence at least about 95% identical thereto;
b) the transgene nucleic acid molecule, wherein the transgene nucleic acid molecule comprises a nucleic acid encoding for a SURF1 polypeptide, comprising a nucleic acid sequence as set forth in SEQ ID NO: 22, or a sequence at least about 90% identical thereto; and
c) a polyA sequence comprising the nucleic acid sequence set forth in SEQ ID NO: 21 or SEQ ID NO: 24, or a sequence at least about 95% identical thereto.

10. The transgene nucleic acid molecule of claim 9, wherein the promoter sequence comprises the nucleic acid sequence set forth in SEQ ID NO: 23;
the transgene nucleic acid molecule comprises a nucleic acid encoding for a SURF1 polypeptide, comprising a nucleic acid sequence as set forth in SEQ ID NO: 22; and
the polyA sequence comprises the nucleic acid sequence set forth in SEQ ID NO: 21, or SEQ ID NO: 24.

11. The transgene nucleic acid molecule of claim 10, wherein administration of the transgene nucleic acid molecule into a cell of a subject causes at least a 20% increase in cytochrome c oxidase (COX) activity in the cell and/or reduces blood lactate elevation (Δlactate) by at least 20%, or more in at least one cell of the subject.

12. The transgene nucleic acid molecule of claim 10, wherein the transgene nucleic acid molecule is for use in gene therapy of SURF1-related Leigh syndrome.

13. A viral vector comprising a transgene nucleic acid molecule encoding a SURF1 polypeptide, wherein the transgene nucleic acid molecule comprises a nucleic acid sequence as set forth in SEQ ID NO: 22, or a nucleic acid sequence at least 90% identical thereto, operably linked to a promoter sequence and a polyA sequence.

14. The viral vector of claim 13, wherein the viral vector is a rAAV viral vector.

15. The viral vector of claim 14, wherein the rAAV viral vector comprises a capsid protein selected from an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein, or an AAVrh.10 capsid protein.

16. The viral vector of claim 15, wherein the rAAV capsid protein is an AAV9 capsid protein.

17. The viral vector of claim 16, wherein the transgene nucleic acid molecule comprises a nucleic acid sequence as set forth in SEQ ID NO: 22, operably linked to a promoter sequence and a polyA sequence.

18. The viral vector of claim 13, comprising in the 5′ to 3′ direction:
a) the promoter sequence, wherein the promoter sequence comprises the nucleic acid sequence set forth in SEQ ID NO: 14 or SEQ ID NO: 23, or a sequence at least about 95% identical thereto;
b) the transgene nucleic acid molecule, wherein the transgene nucleic acid molecule comprises a nucleic acid encoding for a SURF1 polypeptide, comprising a nucleic acid sequence as set forth in SEQ ID NO: 22, or a sequence at least about 90% identical thereto; and
c) the polyA sequence comprising, wherein the polyA sequence comprises the nucleic acid sequence set forth in SEQ ID NO: 21, or SEQ ID NO: 24, or a sequence at least about 95% identical thereto.

19. The viral vector of claim 18, comprising in the 5′ to 3′ direction:
a) a first AAV inverted terminal repeat (ITR) sequence;
b) the promoter sequence;
c) the transgene nucleic acid molecule;
d) the polyA sequence; and
(e) a second AAV ITR sequence,
wherein the vector is packaged in an AAV9 capsid.

20. A method of partially or fully restoring SURF1 gene expression in at least one cell of a subject in need thereof, the method comprising administering the viral vector of claim 13, to the subject.

21. The method of claim 20, wherein administration of the viral vector into the subject restores COX1 activity by at least 20%, or more in at least one cell of the subject, and/or reduces blood lactate elevation (Δlactate) by at least 20%, or more in at least one cell of the subject.

22. The method of claim 20, wherein the subject has or is exhibiting symptoms of a disease and/or disorder related to SURF1 gene.

23. A pharmaceutical composition comprising the transgene nucleic acid of claim 1 and at least one pharmaceutically acceptable excipient and/or additive.

24. A method for treating a subject having a disease and/or disorder related to SURF1 gene, the method comprising administering to the subject a therapeutically effective amount of the pharmaceutical composition of claim 23.

25. The method of claim 24, wherein the disease and/or disorder related to SURF1 gene is SURF1 deficiency, Leigh Syndrome, Mitochondrial complex IV deficiency or Charcot-Marie-Tooth disease 4K.

26. The method of 22, wherein the pharmaceutical composition is administered to the subject orally, transmucosally, inhalationally, transdermally, parenterally, intravenously, subcutaneously, intradermally, intramuscularly, intrapleurally, intracerebrally, intrathecally, intracerebrally, intraventricularly, intranasally, intra-aurally, intra-ocularly, or peri-ocularly, topically, intralymphatically, intracisternally, intranervally or intravitreally.
